SEQUENCE LISTING (20 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 3 - DNA, 1878 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatg

SEQ ID NO: 4 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 5 - DNA, 1573 bp, Homo sapiens
tcgacccacgcgtccgggcagaggaggagggagggagggaaggagcgcggagcccggcccggaagctaggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggcgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaa

SEQ ID NO: 6 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 7 - DNA, 1550 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacctggaagtgctgtttcagggcccgaaacataaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 8 - protein, 397 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHLEVLFQGPKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 9 - DNA, 1538 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacgatccgcaggcgaaacataaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 10 - protein, 393 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHDPQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 11 - DNA, 1881 bp, Homo sapiens
gggcgcagcggggcccgtctgcagcaagtgaccgacggccgggacggccgcctgccccctctgccacctggggcggtgcgggcccggagcccggagcccgggtagcgcgtagagccggcgcgatgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttcctggaagtgctgtttcagggcccgaaacatcagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactagctcctccgagaattcagaccctttggggccaagtttttctggatcctccattgctcgccttggccaggaaccagcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaaaacaacgcataaagaaaaatggccgggccaggtcattggctgggaagtctcagccatgcacggactcgtttccagaggtaattatgagcgcctaccagccaggccacccagccgtgggaggaagggggcgtggcaaggggtgggcacattggtgtctgtgcgaaaggaaaattgacccggaagttcctgtaataaatgtcacaataaaacgaatgaatg

SEQ ID NO: 12 - protein, 433 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFLEVLFQGPGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 13 - DNA, 1279 bp, Homo sapiens
cagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaaaaaaaaaa

SEQ ID NO: 14 - protein, 232 aa, Homo sapiens
MERCPSLGVTLYALVVVLGLRATPAGGQHYLHIRPAPSDNLPLVDLIEHPDPIFDPKEKDLNETLLRSLLGGHYDPGFMATSPPEDRPGGGGGAAGGAEDLAELDQLLRQRPSGAMPSEIKGLEFSEGLAQGKKQRLSKKLRRKLQMWLWSQTFCPVLYAWNDLGSRFWPRYVKVGSCFSKRSCSVPEGMVCKPSKSVHLTVLRWRCQRRGGQRCGWIPIQYPIISECKCSC

SEQ ID NO: 15 - DNA, 3547 bp, Homo sapiens
cccgggtcagcgcccgcccgcccgcgctcctcccggccgctcctcccgccccgcccggcccggcgccgactctgcggccgcccgacgagcccctcgcggcactgccccggccccggccccggccccggccccctcccgccgcaccgcccccggcccggccctccgccctccgcactcccgcctccctccctccgcccgctcccgcgccctcctccctccctcctccccagctgtcccgttcgcgtcatgccgagcctcccggccccgccggccccgctgctgctcctcgggctgctgctgctcggctcccggccggcccgcggcgccggcccagagccccccgtgctgcccatccgttctgagaaggagccgctgcccgttcggggagcggcaggctgcaccttcggcgggaaggtctatgccttggacgagacgtggcacccggacctaggggagccattcggggtgatgcgctgcgtgctgtgcgcctgcgaggcgcctcagtggggtcgccgtaccaggggccctggcagggtcagctgcaagaacatcaaaccagagtgcccaaccccggcctgtgggcagccgcgccagctgccgggacactgctgccagacctgcccccaggagcgcagcagttcggagcggcagccgagcggcctgtccttcgagtatccgcgggacccggagcatcgcagttatagcgaccgcggggagccaggcgctgaggagcgggcccgtggtgacggccacacggacttcgtggcgctgctgacagggccgaggtcgcaggcggtggcacgagcccgagtctcgctgctgcgctctagcctccgcttctctatctcctacaggcggctggaccgccctaccaggatccgcttctcagactccaatggcagtgtcctgtttgagcaccctgcagcccccacccaagatggcctggtctgtggggtgtggcgggcagtgcctcggttgtctctgcggctccttagggcagaacagctgcatgtggcacttgtgacactcactcacccttcaggggaggtctgggggcctctcatccggcaccgggccctggctgcagagaccttcagtgccatcctgactctagaaggccccccacagcagggcgtagggggcatcaccctgctcactctcagtgacacagaggactccttgcattttttgctgctcttccgagggctgctggaacccaggagtgggggactaacccaggttcccttgaggctccagattctacaccaggggcagctactgcgagaacttcaggccaatgtctcagcccaggaaccaggctttgctgaggtgctgcccaacctgacagtccaggagatggactggctggtgctgggggagctgcagatggccctggagtgggcaggcaggccagggctgcgcatcagtggacacattgctgccaggaagagctgcgacgtcctgcaaagtgtcctttgtggggctgatgccctgatcccagtccagacgggtgctgccggctcagccagcctcacgctgctaggaaatggctccctgatctatcaggtgcaagtggtagggacaagcagtgaggtggtggccatgacactggagaccaagcctcagcggagggatcagcgcactgtcctgtgccacatggctggactccagccaggaggacacacggccgtgggtatctgccctgggctgggtgcccgaggggctcatatgctgctgcagaatgagctcttcctgaacgtgggcaccaaggacttcccagacggagagcttcgggggcacgtggctgccctgccctactgtgggcatagcgcccgccatgacacgctgcccgtgcccctagcaggagccctggtgctaccccctgtgaagagccaagcagcagggcacgcctggctttccttggatacccactgtcacctgcactatgaagtgctgctggctgggcttggtggctcagaacaaggcactgtcactgcccacctccttgggcctcctggaacgccagggcctcggcggctgctgaagggattctatggctcagaggcccagggtgtggtgaaggacctggagccggaactgctgcggcacctggcaaaaggcatggcctccctgatgatcaccaccaagggtagccccagaggggagctccgagggcaggtgcacatagccaaccaatgtgaggttggcggactgcgcctggaggcggccggggccgagggggtgcgggcgctgggggctccggatacagcctctgctgcgccgcctgtggtgcctggtctcccggccctagcgcccgccaaacctggtggtcctgggcggccccgagaccccaacacatgcttcttcgaggggcagcagcgcccccacggggctcgctgggcgcccaactacgacccgctctgctcactctgcacctgccagagacgaacggtgatctgtgacccggtggtgtgcccaccgcccagctgcccacacccggtgcaggctcccgaccagtgctgccctgtttgccctgagaaacaagatgtcagagacttgccagggctgccaaggagccgggacccaggagagggctgctattttgatggtgaccggagctggcgggcagcgggtacgcggtggcaccccgttgtgcccccctttggcttaattaagtgtgctgtctgcacctgcaaggggggcactggagaggtgcactgtgagaaggtgcagtgtccccggctggcctgtgcccagcctgtgcgtgtcaaccccaccgactgctgcaaacagtgtccagtggggtcgggggcccacccccagctgggggaccccatgcaggctgatgggccccggggctgccgttttgctgggcagtggttcccagagagtcagagctggcacccctcagtgcccccttttggagagatgagctgtatcacctgcagatgtggggcaggggtgcctcactgtgagcgggatgactgttcactgccactgtcctgtggctcggggaaggagagtcgatgctgttcccgctgcacggcccaccggcggccagccccagagaccagaactgatccagagctggagaaagaagccgaaggctcttagggagcagccagagggccaagtgaccaagaggatggggcctgagctggggaaggggtggcatcgaggaccttcttgcattctcctgtgggaagcccagtgcctttgctcctctgtcctgcctctactcccacccccactacctctgggaaccacagctccacaagggggagaggcagctgggccagaccgaggtcacagccactccaagtcctgccctgccaccctcggcctctgtcctggaagccccacccctttcctcctgtacataatgtcactggcttgttgggatttttaatttatcttcactcagcaccaagggcccccgacactccactcctgctgcccctgagctgagcagagtcattattggagagttttgtatttattaaaacatttctttttcagtcaaaaaaaaaaaaaaaaaaaaaaaaaa

SEQ ID NO: 16 - protein, 955 aa, Homo sapiens
MPSLPAPPAPLLLLGLLLLGSRPARGAGPEPPVLPIRSEKEPLPVRGAAGCTFGGKVYALDETWHPDLGEPFGVMRCVLCACEAPQWGRRTRGPGRVSCKNIKPECPTPACGQPRQLPGHCCQTCPQERSSSERQPSGLSFEYPRDPEHRSYSDRGEPGAEERARGDGHTDFVALLTGPRSQAVARARVSLLRSSLRFSISYRRLDRPTRIRFSDSNGSVLFEHPAAPTQDGLVCGVWRAVPRLSLRLLRAEQLHVALVTLTHPSGEVWGPLIRHRALAAETFSAILTLEGPPQQGVGGITLLTLSDTEDSLHFLLLFRGLLEPRSGGLTQVPLRLQILHQGQLLRELQANVSAQEPGFAEVLPNLTVQEMDWLVLGELQMALEWAGRPGLRISGHIAARKSCDVLQSVLCGADALIPVQTGAAGSASLTLLGNGSLIYQVQVVGTSSEVVAMTLETKPQRRDQRTVLCHMAGLQPGGHTAVGICPGLGARGAHMLLQNELFLNVGTKDFPDGELRGHVAALPYCGHSARHDTLPVPLAGALVLPPVKSQAAGHAWLSLDTHCHLHYEVLLAGLGGSEQGTVTAHLLGPPGTPGPRRLLKGFYGSEAQGVVKDLEPELLRHLAKGMASLMITTKGSPRGELRGQVHIANQCEVGGLRLEAAGAEGVRALGAPDTASAAPPVVPGLPALAPAKPGGPGRPRDPNTCFFEGQQRPHGARWAPNYDPLCSLCTCQRRTVICDPVVCPPPSCPHPVQAPDQCCPVCPEKQDVRDLPGLPRSRDPGEGCYFDGDRSWRAAGTRWHPVVPPFGLIKCAVCTCKGGTGEVHCEKVQCPRLACAQPVRVNPTDCCKQCPVGSGAHPQLGDPMQADGPRGCRFAGQWFPESQSWHPSVPPFGEMSCITCRCGAGVPHCERDDCSLPLSCGSGKESRCCSRCTAHRRPAPETRTDPELEKEAEGS

SEQ ID NO: 17 - DNA, 1140 bp, Homo sapiens
agagcctgtgctactggaaggtggcgtgccctcctctggctggtaccatgcagctcccactggccctgtgtctcgtctgcctgctggtacacacagccttccgtgtagtggagggccaggggtggcaggcgttcaagaatgatgccacggaaatcatccccgagctcggagagtaccccgagcctccaccggagctggagaacaacaagaccatgaaccgggcggagaacggagggcggcctccccaccacccctttgagaccaaagacgtgtccgagtacagctgccgcgagctgcacttcacccgctacgtgaccgatgggccgtgccgcagcgccaagccggtcaccgagctggtgtgctccggccagtgcggcccggcgcgcctgctgcccaacgccatcggccgcggcaagtggtggcgacctagtgggcccgacttccgctgcatccccgaccgctaccgcgcgcagcgcgtgcagctgctgtgtcccggtggtgaggcgccgcgcgcgcgcaaggtgcgcctggtggcctcgtgcaagtgcaagcgcctcacccgcttccacaaccagtcggagctcaaggacttcgggaccgaggccgctcggccgcagaagggccggaagccgcggccccgcgcccggagcgccaaagccaaccaggccgagctggagaacgcctactagagcccgcccgcgcccctccccaccggcgggcgccccggccctgaacccgcgccccacatttctgtcctctgcgcgtggtttgattgtttatatttcattgtaaatgcctgcaacccagggcagggggctgagaccttccaggccctgaggaatcccgggcgccggcaaggcccccctcagcccgccagctgaggggtcccacggggcaggggagggaattgagagtcacagacactgagccacgcagccccgcctctggggccgcctacctttgctggtcccacttcagaggaggcagaaatggaagcattttcaccgccctggggttttaagggagcggtgtgggagtgggaaagtccagggactggttaagaaagttggataagattcccccttgcacctcgctgcccatcagaaagcctgaggcgtgcccagagcacaagactggggg

SEQ ID NO: 18 - protein, 213 aa, Homo sapiens
MQLPLALCLVCLLVHTAFRVVEGQGWQAFKNDATEIIPELGEYPEPPPELENNKTMNRAENGGRPPHHPFETKDVSEYSCRELHFTRYVTDGPCRSAKPVTELVCSGQCGPARLLPNAIGRGKWWRPSGPDFRCIPDRYRAQRVQLLCPGGEAPRARKVRLVASCKCKRLTRFHNQSELKDFGTEAARPQKGRKPRPRARSAKANQAELENAY

SEQ ID NO: 19 - DNA, 1740 bp, Homo sapiens
actcggtgcgccttccgcggaccgggcgacccagtgcacggccgccgcgtcactctcggtcccgctgaccccgcgccgagccccggcggctctggccgcggccgcactcagcgccacgcgtcgaaagcgcaggccccgaggacccgccgcactgacagtatgagccgcacagcctacacggtgggagccctgcttctcctcttggggaccctgctgccggctgctgaagggaaaaagaaagggtcccaaggtgccatccccccgccagacaaggcccagcacaatgactcagagcagactcagtcgccccagcagcctggctccaggaaccgggggcggggccaagggcggggcactgccatgcccggggaggaggtgctggagtccagccaagaggccctgcatgtgacggagcgcaaatacctgaagcgagactggtgcaaaacccagccgcttaagcagaccatccacgaggaaggctgcaacagtcgcaccatcatcaaccgcttctgttacggccagtgcaactctttctacatccccaggcacatccggaaggaggaaggttcctttcagtcctgctccttctgcaagcccaagaaattcactaccatgatggtcacactcaactgccctgaactacagccacctaccaagaagaagagagtcacacgtgtgaagcagtgtcgttgcatatccatcgatttggattaagccaaatccaggtgcacccagcatgtcctaggaatgcagccccaggaagtcccagacctaaaacaaccagattcttacttggcttaaacctagaggccagaagaacccccagctgcctcctggcaggagcctgcttgtgcgtagttcgtgtgcatgagtgtggatgggtgcctgtgggtgtttttagacaccagagaaaacacagtctctgctagagagcactccctattttgtaaacatatctgctttaatggggatgtaccagaaacccacctcaccccggctcacatctaaaggggcggggccgtggtctggttctgactttgtgtttttgtgccctcctggggaccagaatctcctttcggaatgaatgttcatggaagaggctcctctgagggcaagagacctgttttagtgctgcattcgacatggaaaagtccttttaacctgtgcttgcatcctcctttcctcctcctcctcacaatccatctcttcttaagttgatagtgactatgtcagtctaatctcttgtttgccaaggttcctaaattaattcacttaaccatgatgcaaatgtttttcattttgtgaagaccctccagactctgggagaggctggtgtgggcaaggacaagcaggatagtggagtgagaaagggagggtggagggtgaggccaaatcaggtccagcaaaagtcagtagggacattgcagaagcttgaaaggccaataccagaacacaggctgatgcttctgagaaagtcttttcctagtatttaacagaacccaagtgaacagaggagaaatgagattgccagaaagtgattaactttggccgttgcaatctgctcaaacctaacaccaaactgaaaacataaatactgaccactcctatgttcggacccaagcaagttagctaaaccaaaccaactcctctgctttgtccctcaggtggaaaagagaggtagtttagaactctctgcataggggtgggaattaatcaaaaacctcagaggctgaaattcct

SEQ ID NO: 20 - protein, 184 aa, Homo sapiens
MSRTAYTVGALLLLLGTLLPAAEGKKKGSQGAIPPPDKAQHNDSEQTQSPQQPGSRNRGRGQGRGTAMPGEEVLESSQEALHVTERKYLKRDWCKTQPLKQTIHEEGCNSRTIINRFCYGQCNSFYIPRHIRKEEGSFQSCSFCKPKKFTTMMVTLNCPELQPPTKKKRVTRVKQCRCISIDLD

(19) United States
(12) Patent Application Publication
(10) Pub. No.: US 2007/0056050 A1
(43) Pub. Date: Mar. 8, 2007

(54) PRODUCTION OF BONE MORPHOGENIC PROTEINS (BMPs) IN TRANSGENIC MAMMALS

(76) Inventors: Cameron Malcolm Lang Clokie, Toronto (CA); Jeffrey Donald Turner, Chute-A-Blondeau (CA); Sean Alexander Fitzgerald Peel, Oakville (CA)

Correspondence Address: DARBY & DARBY P.C., P. O. BOX 5257, NEW YORK, NY, 10150-5257, US

(21) Appl. No.: 11/427,679

(22) Filed: Jun. 29, 2006

Related U.S. Application Data
(60) Provisional application No. 60/695,263, filed on Jun. 29, 2005.

(51) Int. Cl. A01K 67/027

(52) U.S. Cl. 800/7; 800/14

(57) ABSTRACT

The present invention provides materials and methods for the production of recombinant BMPs in transgenic animals. In particular, the invention provides materials and methods for the production of recombinant BMPs in the milk of transgenic animals that express recombinant BMPs in the mammary gland.

This application claims the benefit of U.S. Provisional application No. 60/695,263 filed Jun. 29, 2005, which is hereby incorporated by reference.

### FIELD OF THE INVENTION

The present invention provides materials and methods for the production of recombinant BMPs in transgenic animals. In particular, the invention provides materials and methods for the production of recombinant BMPs in the milk of transgenic animals that express recombinant BMPs in the mammary gland.

### BACKGROUND OF THE INVENTION

The bone morphogenic proteins (BMPs) are members of the transforming growth factor beta (TGFβ) superfamily of secreted growth and differentiation factors. The BMP subfamily of the TGFβ superfamily comprises at least fifteen proteins, including BMP-2, BMP-3 (also known as osteogenin), BMP-3b (also known as growth and differentiation factor 10, GDF-10), BMP-4, BMP-5, BMP-6, BMP-7 (also known as osteogenic protein-1, OP-1), BMP-8 (also known as osteogenic protein-2, OP-2), BMP-9, BMP-10, BMP-11 (also known as growth and differentiation factor 8, GDF-8, or myostatin), BMP-12 (also known as growth and differentiation factor 7, GDF-7), BMP-13 (also known as growth and differentiation factor 6, GDF-6), BMP-14 (also known as growth and differentiation factor 5, GDF-5), and BMP-15 (for a review, see e.g., Azari etal. Expert Opin Invest Drugs 2001; 10:1677-1686).

BMPs are synthesized as large precursor molecules consisting of an amino terminal signal peptide, a pro-domain, and a carboxy terminal domain harboring the mature protein. The amino-terminal signal peptide and pro-domain regions of the various BMPs vary in size and amino acid sequence, whereas the mature domain shows a greater degree of sequence identity among BMP subfamily members. The mature domain is ordinarily cleaved from the pro-domain by a serine protease, such as furin or plasmin to yield an active mature polypeptide of between 110-140 amino acids in length. The pro-domain appears to be required for normal synthesis and secretion of BMP polypeptides (for a review, see e.g., Clokie et al. Plast Reconstr Surg 2000; 105:628-637; Azari et al. Expert Opin Invest Drugs 2001; 10:1677-1686; and Hoffman et al. Appl Microbiol Biotech 2001; 57:294-308).

The individual members of the BMP family can be divided into several subfamilies within which the sequence of their mature carboxy terminal protein domain is well conserved. BMP-2 and -4 have greater than 90% sequence identity and BMP-5, 6, 7 and 8 have 70 to 90% sequence identity within these subfamilies. Between these 2 groups there is a 55 to 65% sequence identity of the mature proteins. In contrast the mature forms of the TGF-β, the Activin and the Inhibin families share less that 50% sequence identity with these BMPs (Ozkaynak et al. J Biol Chem. 1992; 267:25220-25227).

The highly conserved mature region of BMPs contains seven highly conserved cysteine residues. Six of these cysteine residues are implicated in the formation of intrachain disulfide bonds to form a rigid “cysteine knot” structure. The seventh cysteine is involved in the formation of homodimers and heterodimers via an interchain disulphide bond (for a review, see e.g., Azari et al. Expert Opin Invest Drugs 2001; 10:1677-1686 and Hoffman et al. Appl Microbiol Biotech 2001; 57:294-308).

During intracellular processing, the mature domains of BMPs are cleaved from the pro-domain by furin or plasmin. The mature BMP polypeptides form either homodimers (made up of monomers of a single BMP subfamily member) or heterodimers (made up of monomers of two different BMP subfamily members) connected by one disulfide bond in a head-to-tail arrangement (for a review, see e.g., Azari et al. Expert Opin Invest Drugs 2001; 10:1677-1686 and Hoffman et al. Appl Microbiol Biotech 2001; 57:294-308). Both BMP homodimers (e.g., BMP-2/-2 homodimers) and heterodimers (e.g., BMP-4/-7 heterodimers) are active in vivo (see, e.g., Aono et al. Biochem Biophys Res Comm. 1995; 210:670-677; Kusumoto et al. Biochem Biophys Res Comm 1997; 239:575-579; and Suzuki et al. Biochem Biophys Res Comm 1997; 232:153-156). Under certain conditions, heterodimers of BMP-2, BMP-4, and BMP-7 (e.g., BMP-4/-7 heterodimers and BMP-2/-7 heterodimers) are more active oseoinductive agents than the corresponding homodimers (see, e.g., U.S. Pat. No. 6,593,109 and Aono et al. Biochem Biophys Res Comm. 1995; 210:670-677).

BMPs are glycosylated proteins, with the mature protein having between 1 and 3 potential glycosylation sites (Celeste et al. PNAS 1990; 87:9843-9847). A glycosylation site in the center of the mature protein domain is shared by BMPs 2, 4, 5, 6, 7, and 8 but is absent in BMP-3 (Ozkayanak et al. J. Biol. Chem. 1992; 267:25220-25227). Chemical deglycosylation of BMP-2 and BMP-7 results in reduced activity of these proteins (Sampath et al. J. Biol. Chem. 1990; 265:13198-13205), indicating that proper glycosylation is required for full BMP activity.

Active, mature BMP polypeptides bind to, and initiate a cell signal through, a transmembrane receptor complex formed by types I and II serine/threonine kinase receptor proteins. Type I (BMP receptor-1A or BMP receptor-1B) and Type II (BMP receptor II) receptor proteins are distinguished based upon molecular weight, the presence of a glycine/serine-rich repeat, and the ability to bind to specific ligands. Individual receptors have low affinity binding for BMPs, while heteromeric receptor complexes bind to BMPs with high affinity (for a review, see e.g., Azari et al. Expert Opin Invest Drugs 2001; 10:1677-1686 and Hoffman et al. Appl Microbiol Biotech 2001; 57:294-308).

BMPs also bind to components of the extracellular matrix, and in particular to heparin (see, e.g., Ruppert et al. Eur J Biochem 1996; 237:295-302).

BMPs have been shown to regulate the growth and differentiation of several cell types. They stimulate matrix synthesis in chondroblasts; stimulate alkaline phosphatase activity and collagen synthesis in osteoblasts, induce the differentiation of early mesenchymal progenitors into osteogenic cells (osteoinductive), regulate chemotaxis of monocytes, and regulate the differentiation of neural cells (for a review, see e.g., Azari et al. Expert Opin Invest Drugs 2001; 10:1677-1686 and Hoffman et al. Appl Microbiol Biotech 2001; 57:294-308).

One of the many functions of BMP proteins is to induce cartilage, bone, and connective tissue formation in vertebrates. The most oseoinductive members of the BMP subfamily are BMP-2, BMP-4, BMP-6, BMP-7 and BMP-9 (see, e.g., Hoffman et al. Appl Microbiol Biotech 2001; 57-294-308 and Boden. Orthopaedic Nursing 2005; 24:49-52). This oseoinductive capacity of BMPs has long been considered very promising for a variety of therapeutic and clinical applications, including fracture repair; bone grafts; spine fusion; treatment of skeletal diseases, regeneration of skull, mandibullar, and bone defects; and in oral and dental applications such as dentogenesis and cementogenesis during regeneration of periodontal wounds, bone graft, and sinus augmentation. Currently, recombinant human BMP-2 sold as InFUSE™ by Medtronic and recombinant human BMP-7 sold as OP-1® by Stryker are FDA approved for use in spinal fusion surgery.

Other therapeutic and clinical applications for which BMPs are being developed include Parkinson's and other neurodegenerative diseases, stroke, head injury, cerebral ischemia, liver regeneration, acute and chronic renal injury (see, e.g., Azari et al. Expert Opin Invest Drugs 2001; 10:1677-1686; Hoffman et al. Appl Microbiol Biotech 2001; 57:294-308; Kopp Kidney Int 2002; 61:351-352; and Boden. Orthopaedic Nursing 2005; 24:49-52). BMPs also have potential as veterinary therapeutics and as research or diagnostic reagents (Urist et al. Prog Clin Biol Res. 1985; 187:77-96).

The therapeutic use of BMPs has been hindered by difficulties in obtaining large quantities of pure, active BMP polypeptide, either from endogenous or recombinant sources.

Bone and other tissues contain only low concentrations of mature BMPs and of BMP precursor molecules. Methods exist to extract biologically active BMPs from bone, but these are time consuming methods with non-economical yields: starting from 15 kg raw bone, the final yield is around 0.5 g of partially purified BMPs (Urist et al. Meth Enz 1987; 146:294-312).

Recombinant BMPs have been produced using bacterial expression systems such as E. coli. However, active BMPs are obtained only following an extensive renaturation and dimerization process in vitro. In this process, monomeric BMP must first be purified, then renatured in the presence of chaotropic agents, and finally purified to remove unfolded BMP monomers and other contaminating E. coli proteins. This process is complex, time consuming, and costly, and often has a low yield of active dimer compared to total monomer produced (for a review, see e.g., Hoffman et al. Appl Microbiol Biotech 2001; 57:294-308). Furthermore, BMPs produced by such methods are not glycosylated, and therefore would not be expected to be fully potent.

Attempts at recombinant production of BMP in insect cell culture have resulted in predominantly intracellular BMP accumulation with minimal recovery of active BMP from the supernatant (Maruoka et al. Biochem Mol Biol Int 1995; 35:957-963 and Hazama et al. Biochem Biophys Res Comm 1995; 209:859-866).

Commercially available BMP preparations are based upon mammalian expression systems. Human BMP-2 has been expressed in CHO (Chinese hamster ovary) cells; human BMP-4 has been expressed in a mouse myeloma cell line (NSO) and in a human embryonic kidney cell lines (HEK 292); and human BMP-7 has been expressed in a primate cell line (BS) and in CHO cells (for a review, see e.g., Hoffman et al. Appl Microbiol Biotech 2001; 57:294-308). However, such eukaryotic expression systems generally have lower productivity and yield compared to prokaryotic systems. Due to these low yields, recombinant BMPs are currently very expensive.

Thus a need exists in the art for materials and methods for the production of recombinant, active BMPs on a large scale. In particular, a need exists for materials and methods for efficient, lost-cost production of potent BMPs.

Transgenic animals expressing a protein of interest in the mammary gland have been used for the expression of large quantities (typically 1-10 g/L) of recombinant protein in milk (U.S. Pat. No. 4,873,316; U.S. Pat. No. 5,304,489; U.S. Pat. No. 5,750,172; U.S. Pat. No. 5,831,141; U.S. Pat. No. 6,013,857; U.S. Pat. No. 6,140,552; U.S. Pat. No. 6,268,545; U.S. Pat. No. 6,727,405; PCT Publication No. WO91/08216; PCT Publication No. WO93/25567; PCT Publication No. WO88/01648; Andres et al. Proc Natl Acad Sci USA 1987; 84:1299-1303; Lee, et al. Nucleic Acids Res. 1988; 16:1027-1041; Velander et al. Proc Natl Acad Sci USA 1992; 89:12003-12007; and McClenaghan et al. Biochem. J. 1995; 310:637-641; Gutierrez et al. Transgenic Research 1996; 5:271-279).

To the inventors' knowledge, no attempts to produce recombinant BMPs in the milk of transgenic animals expressing recombinant BMPs in the mammary gland have been reported. This avenue of production of recombinant BMPs has likely not been pursued because BMP is secreted in its processed, biologically active form (Degnin et al. Mol Biol Cell. 2004; 15:5012-20) and thus, it would be expected that such methods would be hampered by problems of ossification of the mammary ducts of such BMP-expressing transgenic animals. The activating protease plasmin is present in milk at all times and increases in concentration as lactation progresses (Politis et al. J. Dairy Sci. 1990; 73:1494-1499 and Turner and Huynh J. Dairy Science 1991; 74:2801-2807).

Further studies indicate that BMP-2 and -4 and their receptors are expressed within the developing mammary gland. In the early stages of mammary gland development, BMP-2 and -4 are expressed in the epithelium and underlying mesenchymal cells, respectively suggesting they play a role in its development (Phippard et al. 1996). It has also been reported that in mice that lack PTHrP, the mammary mesenchyme fails to develop and the morphogenesis of the mammary bud is arrested due to inhibition of MSX-2 (Hens et al. 2005), which in turn results in an increase in BMP activity in the developing mammary gland. Consequently it is likely that transgenic expression of BMPs during mammary development would inhibit their development.

### SUMMARY OF THE INVENTION

The present invention is directed to a non-human transgenic mammal that upon lactation, expresses a recombinant BMP in its milk, wherein the genome of the mammal comprises a nucleic acid sequence encoding a recombinant BMP, optionally a nucleic acid sequence encoding a recombinant BMP-inhibitor, both operably linked to a mammary gland-specific promoter, and a signal sequence that provides secretion of the recombinant BMP and BMP-inhibitor into the milk of the mammal. In preferred embodiments, the mammary gland-specific promoter is a casein promoter. In preferred embodiments, the mammal is a goat. In preferred embodiments, the recombinant BMP is a recombinant human BMP. In preferred embodiments, the recombinant BMP is a recombinant BMP-2 or a recombinant BMP-7. In preferred embodiments, the recombinant BMP-inhibitor is a recombinant human BMP-inhibitor. In preferred embodiments, the recombinant BMP-inhibitor is a recombinant Noggin, Chordin, Sclerostin or Gremlin. In preferred embodiments, the recombinant BMP is a recombinant furin-resistant mutant BMP. In preferred embodiments, the recombinant BMP is a recombinant furin-resistant mutant BMP-2 or recombinant furin-resistant mutant BMP-7. In preferred embodiments, the recombinant BMP is a recombinant BMP/BMP-inhibitor fusion protein. In preferred embodiments, the recombinant BMP is a recombinant BMP/BMP-inhibitor fusion protein comprising BMP-2 and Noggin. In preferred embodiments, the recombinant BMP is a recombinant BMP/BMP-inhibitor fusion protein comprising BMP-7 and Sclerostin.

The present invention is directed to a genetically-engineered nucleic acid sequence, which comprises: (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) at least one signal sequence that provides secretion of the recombinant BMP and BMP-inhibitor. In preferred embodiments, the mammary gland-specific promoter is a casein promoter. In preferred embodiments, the recombinant BMP is a recombinant human BMP. In preferred embodiments, the recombinant BMP is a recombinant BMP-2 or a recombinant BMP-7. In preferred embodiments, the recombinant BMP-inhibitor is a recombinant human BMP-inhibitor. In preferred embodiments, the recombinant BMP-inhibitor is a recombinant Noggin, Chordin, Sclerostin or Gremlin. In preferred embodiments, the recombinant BMP is a recombinant furin-resistant mutant BMP. In preferred embodiments, the recombinant BMP is a recombinant furin-resistant mutant BMP-2 or a recombinant furin-resistant mutant BMP-7. In preferred embodiments, the recombinant BMP is a recombinant BMP/BMP-inhibitor fusion protein. In preferred embodiments, the recombinant BMP is a recombinant BMP/BMP-inhibitor fusion protein comprising BMP-2 and Noggin. In preferred embodiments, the recombinant BMP is a recombinant BMP/BMP-inhibitor fusion protein comprising BMP-7 and Sclerostin.

The present invention is also directed to a mammalian cell which has been transformed to comprise the nucleic acid sequence described above. In preferred embodiments, the cell is selected from the group of embryonic stem cells, embryonal carcinoma cells, primordial germ cells, oocytes, and sperm. In preferred embodiments, the cell is a primary fetal goat cell. In preferred embodiments, the cell is a mammary epithelium cell line.

The present invention is further directed to a non-human mammalian embryo, into which has been introduced the genetically-engineered nucleic acid sequence described above.

The present invention is directed to a method for making a genetically-engineered nucleic acid sequence, which method comprises joining a nucleic acid sequence encoding a recombinant BMP, and optionally a nucleic acid sequence encoding a recombinant BMP-inhibitor, with at least one mammary gland-specific promoter that directs expression of the recombinant BMP and BMP-inhibitor, and with at least one signal sequence that provides secretion of the recombinant BMP and BMP-inhibitor. In preferred embodiments, the recombinant BMP is a recombinant furin-resistant mutant BMP. In preferred embodiments, the recombinant BMP is a recombinant BMP/BMP-inhibitor fusion protein.

The present invention is directed to a method for producing a transgenic non-human mammal that upon lactation secretes a recombinant BMP in its milk, which method comprises allowing an embryo, into which has been introduced a genetically-engineered nucleic acid sequence, comprising (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) atleast one signal sequence that provides secretion of the recombinant BMPand BMP-inhibitor into the milk of the mammal, to grow when transferred into a recipient female mammal, resulting in the recipient female mammal giving birth to the transgenic mammal. In preferred embodiments, the mammary gland-specific promoter is a casein promoter. In preferred embodiments, the embryo is a goat embryo. In preferred embodiments, the recombinant BMP is a recombinant human BMP. In preferred embodiments, the recombinant BMP is a recombinant BMP-2 or a recombinant BMP-7. In preferred embodiments, the recombinant BMP is a recombinant furin-resistant mutant BMP. In preferred embodiments, the recombinant BMP is a recombinant furin-resistant mutant BMP-2 or a recombinant furin-resistant mutant BMP-7. In preferred embodiments, the recombinant BMP is a recombinant BMP/BMP-inhibitor fusion protein. In preferred embodiments, the recombinant BMP is a recombinant BMP/BMP-inhibitor fusion protein comprising BMP-2 and Noggin. In preferred embodiments, the recombinant BMP is a recombinant BMP/BMP-inhibitor fusion protein comprising BMP-7 and Sclerostin. In preferred embodiments, introducing the genetically-engineered nucleic acid sequence into a cell of the embryo, or into a cell that will form at least part of the embryo. In preferred embodiments, introducing the genetically-engineered nucleic acid sequence comprises pronuclear or cytoplasmic microinjection of the genetically-engineered nucleic acid sequence. In preferred embodiments, introducing the genetically-engineered nucleic acid sequence comprises combining a mammalian cell stably transfected with the genetically-engineered nucleic acid sequence with a non-transgenic mammalian embryo. In preferred embodiments, introducing the genetically-engineered nucleic acid sequence comprises the steps of (a) introducing the genetically-engineered nucleic acid sequence into a non-human mammalian oocyte; and (b) activating the oocyte to develop into an embryo.

The present invention is directed to a method for producing a non-human transgenic mammal that upon lactation secretes a recombinant BMP in its milk, which method comprises breeding or cloning a transgenic mammal, the genome of which comprises a genetically-engineered nucleic acid sequence, comprising (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) at least one signal sequence that provides secretion of the recombinant BMP and BMP-inhibitor into the milk of the mammal. In preferred embodiments, the recombinant BMP is a recombinant furin-resistant mutant BMP. In preferred embodiments, the recombinant BMP is a recombinant BMP/BMP-inhibitor fusion protein.

The present invention is directed to a method for producing a recombinant BMP, which method comprises: (a) inducing or maintaining lactation of a transgenic mammal, the genome of which comprises a nucleic acid sequence encoding a recombinant BMP, optionally a recombinant BMP-inhibitor, both operably linked to a mammary gland-specific promoter, wherein the sequence further comprises a signal sequence that provides secretion of the recombinant BMP and BMP-inhibitor into the milk of the mammal; and (b) extracting milk from the lactating mammal. In preferred embodiments, the method comprises the additional steps of: (a) optional proteolytic cleavage of the recombinant BMP; and (b) purifying the recombinant BMP from the extracted milk.

The present invention is directed to the milk of a non-human mammal comprising a recombinant BMP. In preferred embodiments, the milk is whole milk. In preferred embodiments, the milk is defatted milk.

The present invention is directed to a method for producing a recombinant BMP in a culture of mammary epithelium cells, which method comprises: (a) culturing said cells, into which a nucleic acid sequence comprising (i) a nucleic acid sequence encoding a recombinant BMP, (ii) a mammary gland-specific promoter that directs expression of the recombinant BMP within said cells, and (iii) a signal sequence that provides secretion of the recombinant BMP into the cell culture medium, has been introduced; (b) culturing the cells; and (c) collecting the cell culture medium of the cell culture. In preferred embodiments, the method employs the additional steps of: (a) optional proteolytic cleavage of the recombinant BMP; and (b) purifying the recombinant BMP from the collected cell culture medium. In preferred embodiments, the mammary epithelium cells are MAC-T cells (ATCC Number CRL 10274). In preferred embodiments, the mammary epithelium cells are 184B5 cells (ATCC Number CRL-8799), 184A1 cells (ATCC Number CRL-8798), MCF7 cells (ATCC Number HTB-22), or ZR-75-30 cells (ATCC Number CRL-1504).

The present invention is directed to cell culture medium comprising a recombinant BMP produced by cultured mammary epithelium cells.

The present invention is directed to a protein comprising a recombinant BMP containing: (a) a mutated furin proteolytic cleavage sequence such that the protein is resistant to proteolytic cleavage by furin or furin-like proteases; and (b) a non-furin proteolytic cleavage sequence such that the protein is susceptible to proteolytic cleavage. In preferred embodiments, the protein does not have BMP activity. In preferred embodiments, the protein has BMP activity after proteolytic cleavage. In preferred embodiments, the protein is recombinant BMP-2, recombinant BMP-4 or recombinant BMP-7, or a homodimer or heterodimer thereof. In preferred embodiments, the protein is a recombinant human BMP-2, recombinant human BMP-4 or recombinant human BMP-7, or a homodimer or heterodimer thereof.

The present invention is directed to a fusion protein comprising a recombinant BMP, a recombinant BMP-inhibitor and a linker region containing at least one proteolytic cleavage site. In preferred embodiments, the protein does not have BMP activity. In preferred embodiments, the protein has BMP activity after proteolytic cleavage. In preferred embodiments, the recombinant BMP is recombinant BMP-2, and the recombinant BMP-inhibitor is a recombinant Noggin. In preferred embodiments, the recombinant BMP is recombinant BMP-7, and the recombinant BMP-inhibitor is a recombinant Sclerostin. In preferred embodiments, the recombinant BMP is a recombinant human BMP-2, and the recombinant BMP-inhibitor is a recombinant human Noggin. In preferred embodiments, the recombinant BMP is a recombinant human BMP-7, and the recombinant BMP-inhibitor is a recombinant human Sclerostin.

The present invention is directed to a method for producing a pharmaceutical composition, which comprises combining (a) a recombinant BMP produced by a transgenic mammal with (b) a pharmaceutically acceptable carrier or excipient.

The present invention is directed to a method for producing a pharmaceutical composition, which comprises combining (a) a recombinant BMP produced in a culture of mammary epithelium cells with (b) a pharmaceutically acceptable carrier or excipient.

The present invention is directed to a non-human transgenic mammal that upon lactation, expresses a recombinant BMP in its milk, wherein the genome of the mammal comprises (a) a first nucleic acid sequence encoding a first recombinant BMP, operably linked to a first mammary gland-specific promoter, and a first signal sequence that provides secretion of the first recombinant BMP into the milk of the mammal; (b) a second nucleic acid sequence encoding a second recombinant BMP, operably linked to a second mammary gland-specific promoter, and a second signal sequence that provides secretion of the second recombinant BMP into the milk of the mammal; and (c) optionally a third nucleic acid sequence encoding a recombinant BMP-inhibitor, operably linked to a third mammary gland-specific promoter, and a third signal sequence that provides secretion of the recombinant BMP-inhibitor into the milk of the mammal. In preferred embodiments, the first mammary gland-specific promoter, the second mammary gland-specific promoter and the third mammary gland-specific promoter are casein promoters. In preferred embodiments, the mammal is a goat. In preferred embodiments, the first recombinant BMP is a recombinant human BMP, the second recombinant BMP is a recombinant human BMP and the recombinant BMP-inhibitor is a recombinant human BMP-inhibitor. In preferred embodiments, the first recombinant BMP is a recombinant BMP-2, the second recombinant BMP is a recombinant BMP-7 and the recombinant BMP-inhibitor is a recombinant Gremlin.

The present invention is directed to a method for producing a non-human transgenic mammal that upon lactation secretes a recombinant BMP in its milk, which method comprises allowing an embryo, into which has been introduced a first genetically-engineered nucleic acid sequence, a second genetically-engineered nucleic acid sequence and optionally a third genetically-engineered nucleic acid sequence, to grow when transferred into a recipient female mammal, resulting in the recipient female mammal giving birth to the transgenic mammal, wherein the first genetically-engineered nucleic acid sequence comprises (i) a first nucleic acid sequence encoding a first recombinant BMP; (ii) a first mammary gland-specific promoter that directs expression of the first recombinant BMP; and (iii) a first signal sequence that provides secretion of the first recombinant BMP into the milk of the mammal, and wherein the second genetically-engineered nucleic acid sequence comprises (i) a second nucleic acid sequence encoding a second recombinant BMP; (ii) a second mammary gland-specific promoter that directs expression of the second recombinant BMP; and (iii) a second signal sequence that provides secretion of the second recombinant BMP into the milk of the mammal, and wherein the third genetically-engineered nucleic acid sequence comprises (i) a third nucleic acid sequence encoding a recombinant BMP-inhibitor; (ii) a third mammary gland-specific promoter that directs expression of the recombinant BMP-inhibitor; and (iii) a third signal sequence that provides secretion of the recombinant BMP-inhibitor into the milk of the mammal. In preferred embodiments, the first mammary gland-specific promoter, the second mammary gland-specific promoter and the third mammary gland-specific promoter are casein promoters. In preferred embodiments, the embryo is a goat embryo. In preferred embodiments, the first recombinant BMP is a recombinant human BMP, the second recombinant BMP is a recombinant human BMP and the recombinant BMP-inhibitor is a recombinant human BMP-inhibitor. In preferred embodiments, the first recombinant BMP is a recombinant BMP-2, the second recombinant BMP is a recombinant BMP-7 and the recombinant BMP-inhibitor is a recombinant Gremlin. In preferred embodiments, introducing the first, second or third genetically-engineered nucleic acid sequence into a cell of the embryo, or into a cell that will form at least part of the embryo. In preferred embodiments, introducing the first genetically-engineered nucleic acid sequence into a cell of the embryo, or into a cell that will form at least part of the embryo, introducing the second genetically-engineered nucleic acid sequence into a cell of the embryo, or into a cell that will form at least part of the embryo and introducing the third genetically-engineered nucleic acid sequence into a cell of the embryo, or into a cell that will form at least part of the embryo. In preferred embodiments, introducing the first, second or third genetically-engineered nucleic acid sequence comprises pronuclear or cytoplasmic microinjection of the first, second or third genetically-engineered nucleic acid sequence. In preferred embodiments, introducing the first, second or third genetically-engineered nucleic acid sequence comprises combining a mammalian cell stably transfected with the first, second or third genetically-engineered nucleic acid sequence with a non-transgenic mammalian embryo. In preferred embodiments, introducing the first, second or third genetically-engineered nucleic acid sequence comprises the steps of (a) introducing the first, second or third genetically-engineered nucleic acid sequence into a non-human mammalian oocyte; and (b) activating the oocyte to develop into an embryo.

The present invention is directed to a method for producing a non-human transgenic mammal that upon lactation secretes a recombinant BMP in its milk, which method comprises breeding a first transgenic mammal, the genome of which comprises a first genetically-engineered nucleic acid sequence, comprising (i) a first nucleic acid sequence encoding a first recombinant BMP; (ii) a first mammary gland-specific promoter that directs expression of the first recombinant BMP; and (iii) a first signal sequence that provides secretion of the first recombinant BMP into the milk of the mammal, to a second transgenic mammal, the genome of which comprises a second genetically-engineered nucleic acid sequence, comprising (i) a second nucleic acid sequence encoding a second recombinant BMP; (ii) a second mammary gland-specific promoter that directs expression of the second recombinant BMP; and (iii) a second signal sequence that provides secretion of the second recombinant BMP into the milk of the mammal. In preferred embodiments, the first mammary gland-specific promoter and the second mammary gland-specific promoter are casein promoters. In preferred embodiments, the first transgenic animal and the second transgenic animal are goats. In preferred embodiments, the first recombinant BMP and the second recombinant BMP are recombinant human BMPs. In preferred embodiments, the first recombinant BMP is a recombinant BMP-2 and the second recombinant BMP is a recombinant BMP-7. In preferred embodiments, the first recombinant BMP and the second recombinant BMP are recombinant furin-resistant mutant BMPs. In preferred embodiments, the first recombinant BMP and the second recombinant BMP are recombinant BMP/BMP-inhibitor fusion proteins.

The present invention is directed to a method for producing a transgenic mammal that upon lactation secretes a recombinant BMP and BMP-inhibitor in its milk, which method comprises breeding a first transgenic mammal, the genome of which comprises a first genetically-engineered nucleic acid sequence, comprising (i) a first nucleic acid sequence encoding a recombinant BMP; (ii) a first mammary gland-specific promoter that directs expression of the recombinant BMP; and (iii) a first signal sequence that provides secretion of the recombinant BMP into the milk of the mammal to a second transgenic mammal, the genome of which comprises a second genetically-engineered nucleic acid sequence, comprising (i) a second nucleic acid sequence encoding a recombinant BMP-inhibitor; and (ii) a second mammary gland-specific promoter that directs expression of the recombinant BMP-inhibitor; and (iii) a second signal sequence that provides secretion of the recombinant BMP-inhibitor into the milk of the mammal. In preferred embodiments, the first mammary gland-specific promoter and the second mammary gland-specific promoter are casein promoters. In preferred embodiments, the first transgenic animal and the second transgenic animal are goats. In preferred embodiments, the recombinant BMP is a recombinant human BMP and the recombinant BMP-inhibitor is a recombinant human BMP-inhibitor. In preferred embodiments, the recombinant BMP is a recombinant BMP-2 and the recombinant BMP-inhibitor is a recombinant Noggin. In preferred embodiments, the recombinant BMP is a recombinant BMP-7 and the recombinant BMP-inhibitor is a recombinant Sclerostin.

### DESCRIPTION OF THE DRAWINGS

FIG. 1 depicts an exemplary nucleotide sequence for a human BMP-2 (SEQ ID NO: 1) derived from GenBank Accession number M22489.1.

FIG. 2 depicts an exemplary amino acid sequence for a human BMP-2 (SEQ ID NO: 2) derived from GenBank Accession number AAA51834.1.

FIG. 3 depicts an exemplary nucleotide sequence for a human BMP-7 (SEQ ID NO: 3) derived from GenBank Accession number NM_{—}001719.1.

FIG. 4 depicts an exemplary amino acid sequence for a human BMP-7 (SEQ ID NO: 4) derived from GenBank Accession number NP_{—}001719.1.

FIG. 5 depicts an exemplary nucleotide sequence for a human BMP-4 (SEQ ID NO: 5) derived from GenBank Accession number BC020546.2.

FIG. 6 depicts an exemplary amino acid sequence for a human BMP-4 (SEQ ID NO: 6) derived from GenBank Accession number AAH20546.1.

FIG. 7 depicts an exemplary nucleotide sequence encoding a human BMP-2 with a mutated furin resitant PreScission cleavage site (SEQ ID NO: 7) derived originally from GenBank Accession number NM_{—}001200.1.

FIG. 8 depicts an exemplary amino acid sequence for a human BMP-2 with a mutated furin resitant PreScission cleavage site (SEQ ID NO: 8) derived originally from GenBank Accession number NP_{—}001191.1.

FIG. 9 depicts an exemplary nucleotide sequence for a human BMP-2 with a mutated furin resitant acid labile cleavage site (SEQ ID NO: 9) derived originally from GenBank Accession number NM_{—}001200.1.

FIG. 10 depicts an exemplary amino acid sequence for a human BMP-2 with a mutated furin resistant acid labile cleavage site (SEQ ID NO: 10) derived originally from GenBank Accession number NP_{—}001191.1.

FIG. 11 depicts an exemplary nucleotide sequence for a human BMP-7 with a mutated furin resitant PreScission cleavage site (SEQ ID NO: 11) derived originally from GenBank Accession number NM_{—}001719.1.

FIG. 12 depicts an exemplary amino acid sequence for a human BMP-7 with a mutated furin resitant PreScission cleavage site (SEQ ID NO: 12) derived originally from GenBank Accession number NP_{—}001710.1.

FIG. 13 depicts an exemplary nucleotide sequence for a human Noggin (SEQ ID NO: 13) derived from GenBank Accession number NM_{—}005450.2.

FIG. 14 depicts an exemplary amino acid sequence for a human Noggin (SEQ ID NO: 14) derived from GenBank Accession number NP_{—}005441.1.

FIG. 15 depicts an exemplary nucleotide sequence for a human Chordin (SEQ ID NO: 15) derived from GenBank Accession number NM_{—}003741.2.

FIG. 16 depicts an exemplary amino acid sequence for a human Chordin (SEQ ID NO: 16) derived from GenBank Accession number NP_{—}003732.2.

FIG. 17 depicts an exemplary nucleotide sequence for a human Sclerostin (SEQ ID NO: 17) derived from GenBank Accession number NM_{—}025237.2.

FIG. 18 depicts an exemplary amino acid sequence for a human Sclerostin (SEQ ID NO: 18) derived from GenBank Accession number NP_{—}079513.1.

FIG. 19 depicts an exemplary nucleotide sequence for a human Gremlin (SEQ ID NO: 19) derived from GenBank Accession number NM_{—}013372.5.

FIG. 20 depicts an exemplary amino acid sequence for a human Gremlin (SEQ ID NO: 20) derived from GenBank Accession number NP_{—}037504.1.

FIG. 21 depicts an exemplary micro-CT image of ectopic bone induced in the thigh muscle of a mouse by an implant possessing BMP activity.

FIG. 22 depicts an exemplary histological section through an ossicle of ectopic bone induced in the thigh muscle of a mouse by an implant possessing BMP activity.

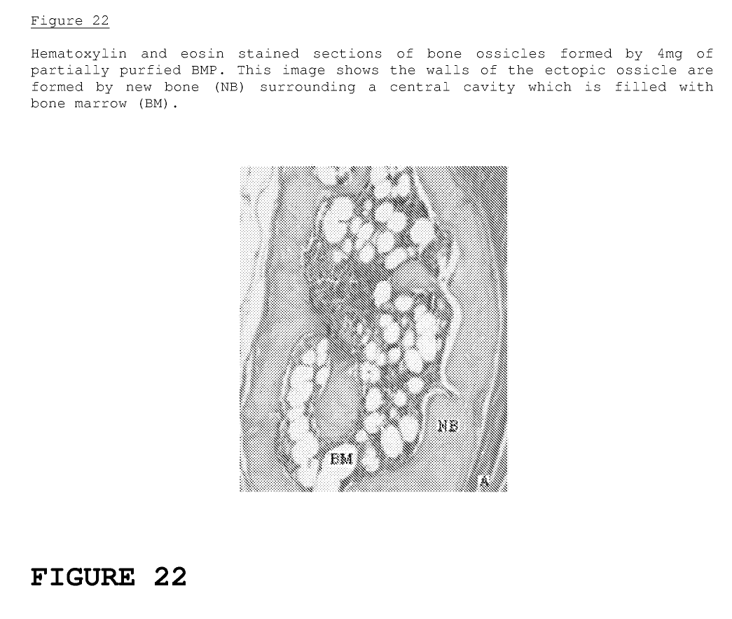

### DETAILED DESCRIPTION

The present inventors have discovered methods for producing large quantities of recombinant BMPs in the milk of lactating transgenic mammals. The methods of the invention allow for rapid, cost-effective production of large quantities of recombinant BMPs. Such recombinant BMPs may be used for a variety of therapeutic and clinical applications, including fracture repair; bone grafts; spine fusion; treatment of skeletal diseases, regeneration of skull, mandibullar, and bone defects; oral and dental applications such as dentogenesis and cementogenesis during regeneration of periodontal wounds, bone graft, and sinus augmentation; Parkinson's and other neurodegenerative diseases; stroke; head injury; cerebral ischemia; liver regeneration; and acute and chronic renal injury.

Definitions

As used herein, the terms “bone morphogenic protein” or “BMP” are used interchangeably and refer to any member of the bone morphogenic protein (BMP) subfamily of the transforming growth factor beta (TGFβ) superfamily of growth and differentiation factors, including BMP-2, BMP-3 (also known as osteogenin), BMP-3b (also known as growth and differentiation factor 10, GDF-10), BMP-4, BMP-5, BMP-6, BMP-7 (also known as osteogenic protein-1, OP-1), BMP-8 (also known as osteogenic protein-2, OP-2), BMP-9, BMP-10, BMP-11 (also known as growth and differentiation factor 8, GDF-8, or myostatin), BMP-12 (also known as growth and differentiation factor 7, GDF-7), BMP-13 (also known as growth and differentiation factor 6, GDF-6), BMP-14 (also known as growth and differentiation factor 5, GDF-5), and BMP-15.

BMP subfamily members contain an amino terminal signal peptide of variable size, a pro-domain of variable size, and a carboxy terminal mature protein domain of approximately 110 to 140 amino acids in length that contains seven conserved cysteine residues.

Generally speaking, the individual members of the BMP family are highly conserved proteins having at least 50% sequence identity, preferably at least 70% sequence identity, and more preferably at least 90% sequence identity to each other. In particular, the individual members of the BMP family have a highly conserved carboxy terminal mature protein domain having at least 50% sequence identity, preferably at least 70% sequence identity, and more preferably at least 90% sequence identity, between the different family members.

The terms “bone morphogenic protein” and “BMP” also encompass allelic variants of BMPs, function conservative variants of BMPs, and mutant BMPs that retain BMP activity. The BMP activity of such variants and mutants may be confirmed by any of the methods well known in the art (see the section Assays to characterize BMP, below) or as described in Example 4.

The nucleotide and amino acid sequences for BMP orthologs from a variety of species (including human, mouse, rat, cow, rabbit, dog, chicken, turtle, tilapia, zebrafish and Xenopus) are known in the art. For example, nucleotide and amino acid sequences for a human BMP-2 (see, for example, Wozney et al. Science 1988; 242:1528-1534), BMP-3 (see, for example, Wozney et al. Science 1988; 242:1528-1534), BMP-3b (see, for example, Hino et al. Biochem. Biophys. Res. Commun. 1996; 223:304-310), BMP-4 (see, for example, Oida et al. DNA Seq. 1995; 5:273-275), BMP-5 (see, for example, Celeste et al. Proc Natl Acad Sci USA 1990; 87:9843-9847), BMP-6 (see, for example, Celeste et al. Proc Natl Acad Sci USA 1990; 87:9843-9847), BMP-7 (see, for example, Celeste et al. Proc Natl Acad Sci USA 1990; 87:9843-9847), BMP-8 (see, for example, Ozkaynak J. Biol. Chem. 1992; 267:25220-25227), BMP-9 (see, for example, Strausberg et al. Proc Natl Acad Sci USA 2002; 99:16899-16903), BMP-10 (see, for example, Neuhaus et al. Mech. Dev. 1999; 80:181-184); BMP-11 (see, for example, Gonzalez-Cadavid et al. Proc Natl Acad Sci USA 1998; 95:14938-14943); BMP-12 (see, for example, U.S. Pat. No. 5,658,882), BMP-13 (see, for example, U.S. Pat. No. 5,658,882), BMP-14 (see, for example, Chang et al. J. Biol. Chem. 1994; 269:28227-28234), and BMP-15 (see, for example, Dube et al Mol. Endocrinol. 1998; 12:1809-1817) have been reported.

In preferred embodiments, the BMP is BMP-2, BMP-4, BMP-6, BMP-7, or BMP-9. In particularly preferred embodiments the BMP is BMP-2, BMP-4 or BMP-7.

In preferred embodiments the BMP is a mammalian BMP (e.g., mammalian BMP-2 or mammalian BMP-7). In particularly preferred embodiments, the BMP is a human BMP (hBMP) (e.g. hBMP-2 or hBMP-7).

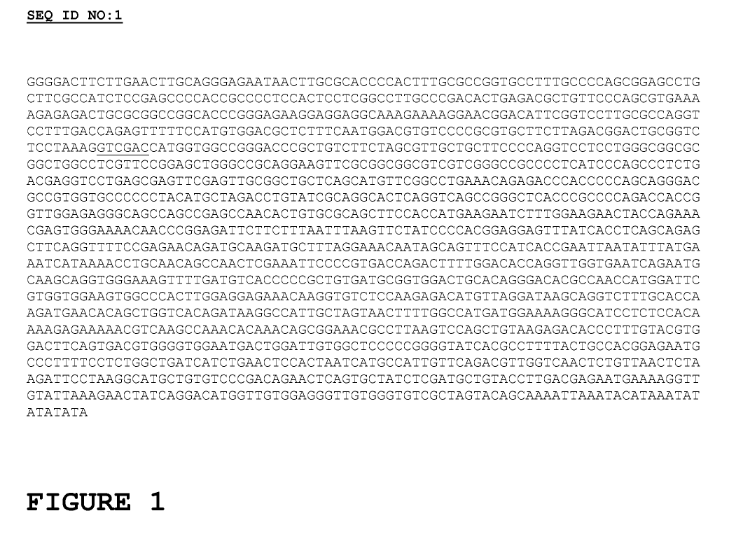

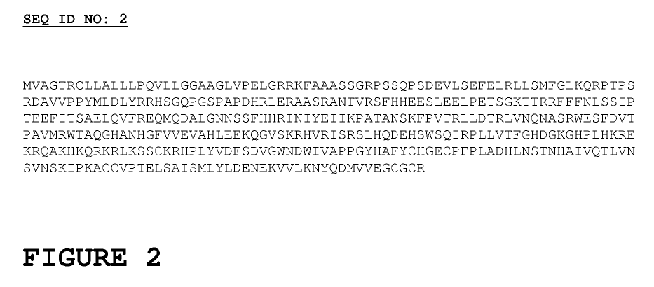

Amino acid and nucleotide sequences for BMP-2 have been reported for a variety of species, including human, mouse, rat, rabbit, dog, chicken, turtle, zebrafish and Xenopus. In preferred embodiments, BMP-2 is a mammalian BMP-2. In particularly preferred embodiments, BMP-2 is a human BMP-2 (hBMP-2). Exemplary nucleotide and amino acid sequences for human BMP-2 are set forth in SEQ ID NOs: 1 and 2, respectively (see FIG. 1 and FIG. 2).

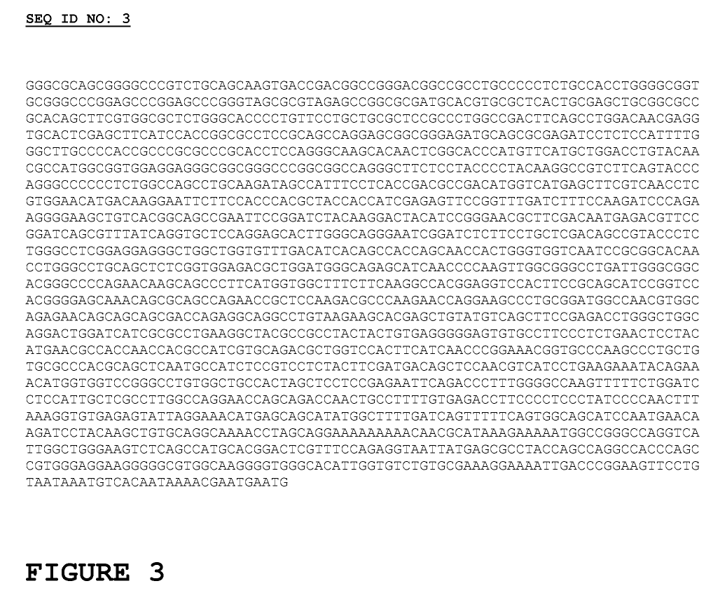

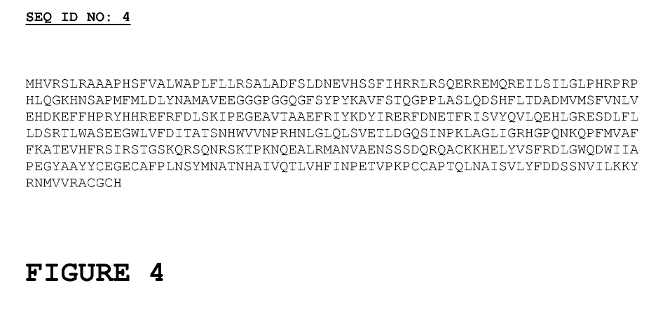

Amino acid and nucleotide sequences for BMP-7 (also known as or OP-1) have been reported for a variety of species, including human, mouse, rat, pig, chicken, Xenopus, and zebrafish. In preferred embodiments, BMP-7 is a mammalian BMP-7. In particularly preferred embodiments, BMP-7 is a human BMP-7 (hBMP-7). Exemplary nucleotide and amino acid sequences for human BMP-7 are set forth in SEQ ID NOs: 3 and 4, respectively (see FIG. 3 and FIG. 4).

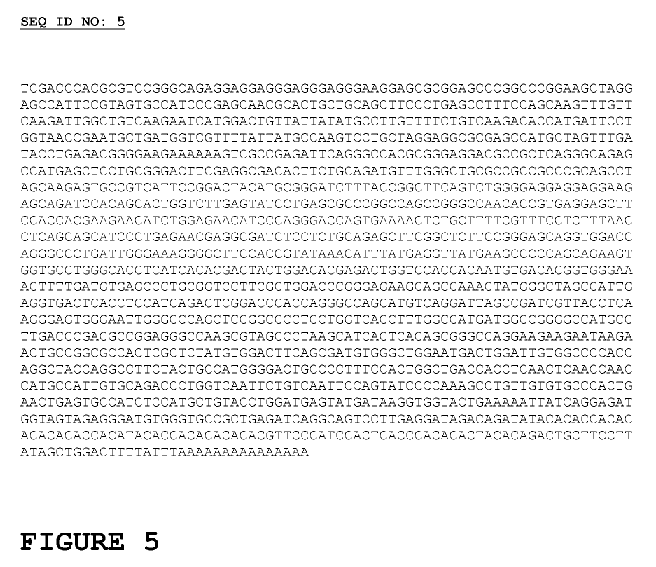

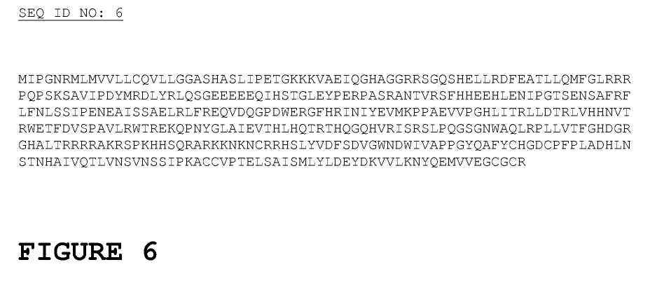

Amino acid and nucleotide sequences for BMP-4 have been reported for a variety of species, including human, cow, sheep, dog, rat, rabbit, mouse, chicken, Xenopus, and zebrafish. In preferred embodiments, BMP-4 is a mammalian BMP-4. In particularly preferred embodiments, BMP-4 is a human BMP-4 (hBMP-4). Exemplary nucleotide and amino acid sequences for human BMP-4 are set forth in SEQ ID NOs: 5 and 6, respectively (see FIG. 5 and FIG. 6).

By “recombinant bone morphogenic protein” or “recombinant BMP” is meant a BMP, a furin-resistant mutant BMP or a BMP/BMP-inhibitor fusion protein produced by a transiently transfected, stably transfected, or transgenic host cell or animal as directed by one of the expression constructs of the invention. The term “recombinant BMP” encompasses BMP, furin-resistant mutant BMP and BMP/BMP-inhibtior proteins in monomeric, homodimeric, and heterodimeric forms. In preferred embodiments, the recombinant BMP is a homodimer or a heterodimer. In preferred embodiments, the recombinant BMP has a glycosylation profile that is substantially similar to that of the corresponding native BMP. The term “recombinant BMP” also encompasses pharmaceutically acceptable salts of such a polypeptide.

By “proteolytic cleavage sequence” or “proteolytic cleavage site” is meant the amino acid sequence of a peptide or protein that either serves as a recognition sequence for specific enzymatic protease cleavage, or renders the peptide or protein susceptible to non-enzymatic proteolytic cleavage under suitable conditions such as treatment with acids or bases. Proteolytic cleavage of a peptide or protein can be performed either prior to, or after isolation of the protein from its expression host or media.

By “pro-domain” or “pro-domain sequence” or “pro” sequence is meant the protein sequence comprising the regulatory N-terminal sequence of the TGF-β family members, including all BMPs.

By “proBMP” is meant a BMP that is covalently and operably linked to its pro-domain.

By “recombinant proBMP” is meant a proBMP that is produced by a transiently transfected, stably transfected, or transgenic host cell or animal as directed by one of the expression constructs of the invention.

By “furin-resistant mutant BMP” (frm-BMP) is meant a proBMP protein with an altered pro-domain amino acid sequence such that the native furin protease cleavage site (R-X-X-R ↓) is mutated in order to prevent protease cleavage by furin, or furin-like proteases, and facilitate cleavage by a different protease enzyme, including those described in Table 1 or by mild acid hydrolysis such as by the acid labile aspartyl-proline sequence.

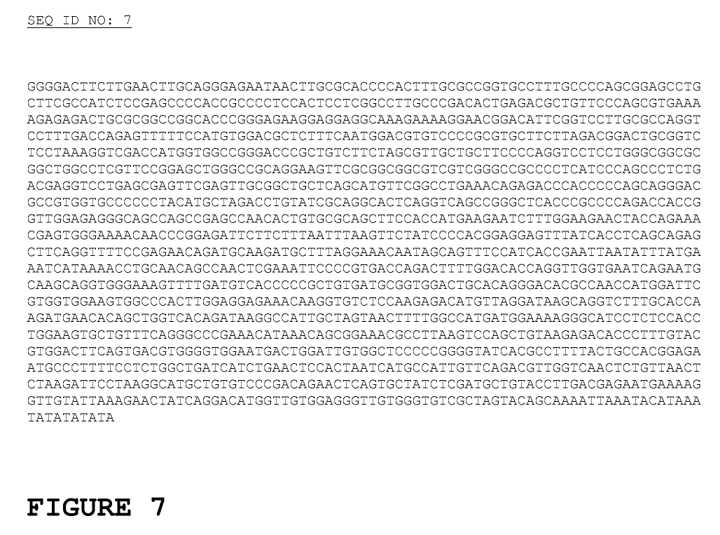

The nucleic acid sequences encoding representative furin-resistant mutant BMPs and their corresponding amino acid sequences are shown in FIGS. 7-12. These sequences illustrate the invention by way of example, and not by way of limitation.

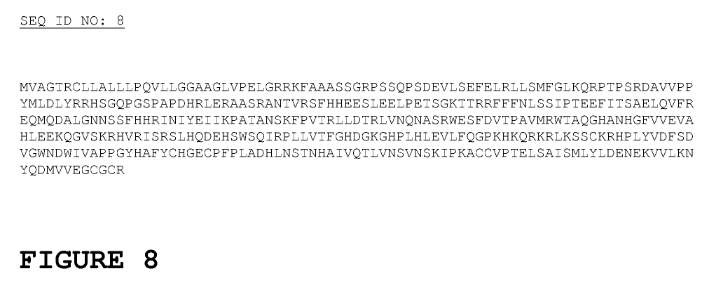

FIG. 8 shows a BMP-2 amino acid sequence wherein the furin-cleavage site KREKRˆQAKH has been changed to LEVLFQˆGPKH, which is an amino acid sequence recognized and selectively proteolyzed by the PreScission protease. The amino acid sequence is marked with a “ˆ” to denote the site of cleavage. The corresponding nucleotide sequence is shown FIG. 7.

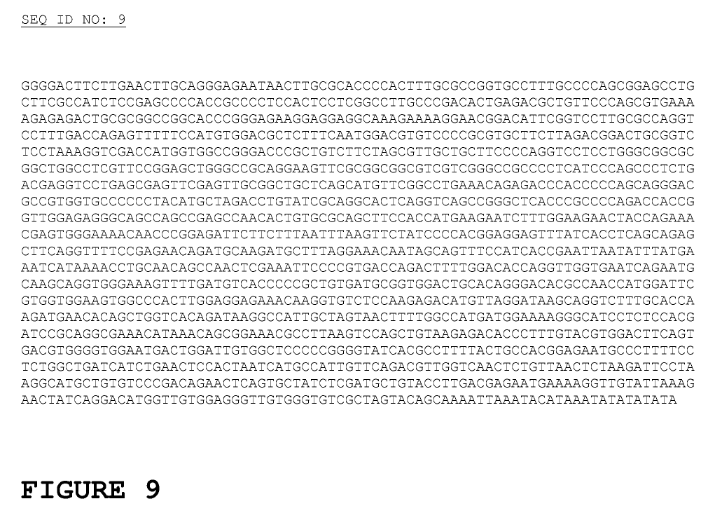

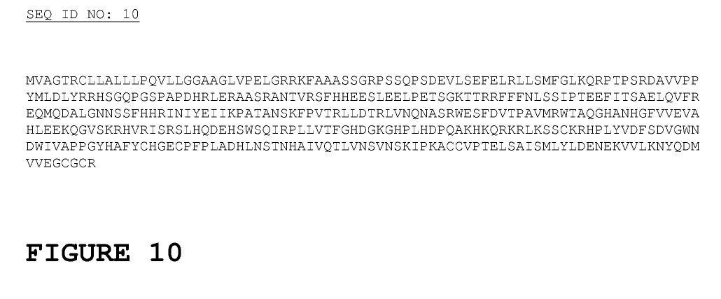

FIG. 10 shows a BMP-2 amino acid sequence wherein the furin-cleavage site KREKRˆQAKH has been changed to DˆPQAKH, which is an acid sensitive amino acid sequence known to be cleaved under acidic conditions. The amino acid sequence is marked with a “ˆ” to denote the site of cleavage. The corresponding nucleotide sequence is shown FIG. 9.

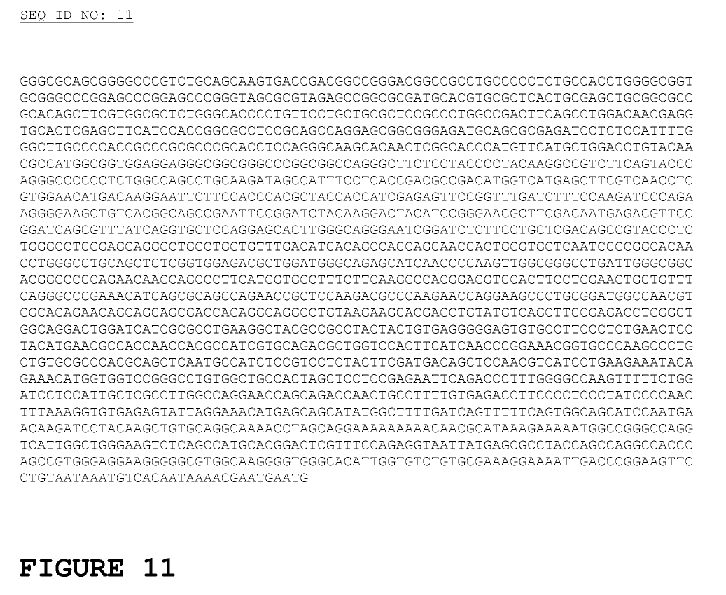

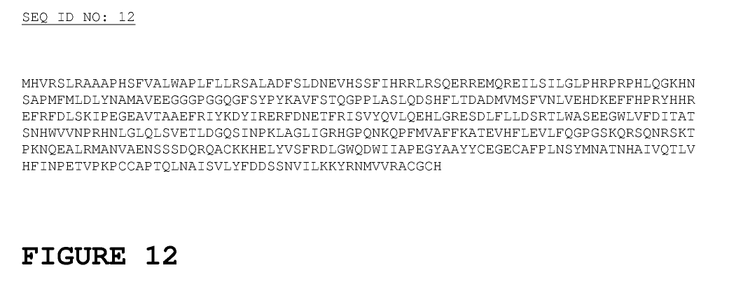

FIG. 12 shows a BMP-7 amino acid sequence wherein the furin-cleavage site RSIRˆSTGSK has been changed to LEVLFQˆGPKH, which is an amino acid sequence recognized and selectively proteolyzed by the PreScission protease. The amino acid sequence is marked with a “ˆ” to denote the site of cleavage. The corresponding nucleotide sequence is shown FIG. 11.

By “recombinant furin-resistant mutant BMP” is meant a furin-resistant mutant BMP produced by a transiently transfected, stably transfected, or transgenic host cell or animal as directed by one of the expression constructs of the invention. The term “recombinant furin-resistant mutant BMP” encompasses furin-resistant mutant BMP proteins in monomeric, homodimeric, and heterodimeric forms. In preferred embodiments, the recombinant frm-BMP has the furin cleavage site mutated into a sequence that is resistant to furin cleavage but is cleavable by another protease or by mild acid hydrolysis. In preferred embodiments the mutated site is mutated to the cleavage site for the PreScission enzyme. In another preferred embodiment the furin site amino acids are mutated into the acid labile aspartyl-proline residues. In preferred embodiments the frm-BMP has a glycosylation profile that is substantially similar to that of the corresponding native BMP.

By “bone morphogenic protein inhibitor”, “BMP-inhibitor” or “BMP binding protein” is meant a protein, or protein fragment thereof, with the ability to bind and/or inhibit the activity of a bone morphogenic protein (BMP) family member, such that the active BMP can be recovered after purification, or in the case of fused inhibitors proteolytic cleavage and purification. The terms “BMP-inhibitor” or “BMP-binding protein” also encompass allelic variants, function conservative variants, mutant BMP inhibitors and fragments thereof that retain BMP binding and/or inhibitory activity. Included are Noggin, Chordin, DAN, Gremlin, Sclerostin, USAG-1, Follistatin, A2HS/fetuin as well as nucleic acid encodeable synthetic peptide-based BMP-inhibitors created by either protein design or random or combinatorial mutagenesis coupled with selection. The terms “BMP-inhibitor” or “BMP-binding protein” encompass BMP-inhibitor proteins in monomeric, homodimeric, heterodimeric and fused or chimeric forms.

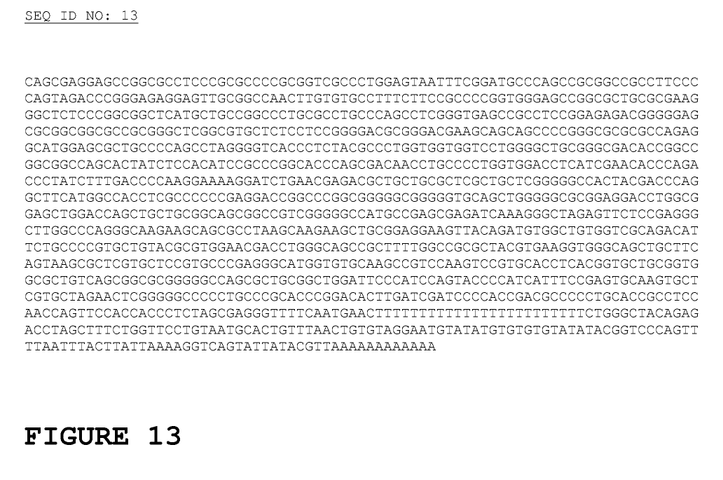

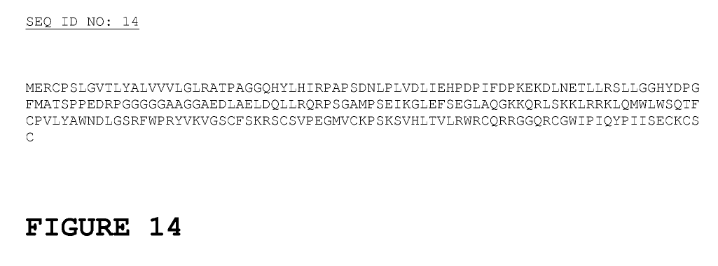

In a preferred embodiment, the BMP-inhibitor is Noggin. Amino acid and nucleotide sequences for Noggin have been reported for a variety of species, including human, mouse, rat, and chicken. In preferred embodiments, Noggin is a mammalian Noggin. In particularly preferred embodiments, Noggin is a human Noggin (hNoggin). Exemplary nucleotide and amino acid sequences for human Noggin are set forth in SEQ ID NOs: 13 and 14, respectively (see FIG. 13 and FIG. 14).

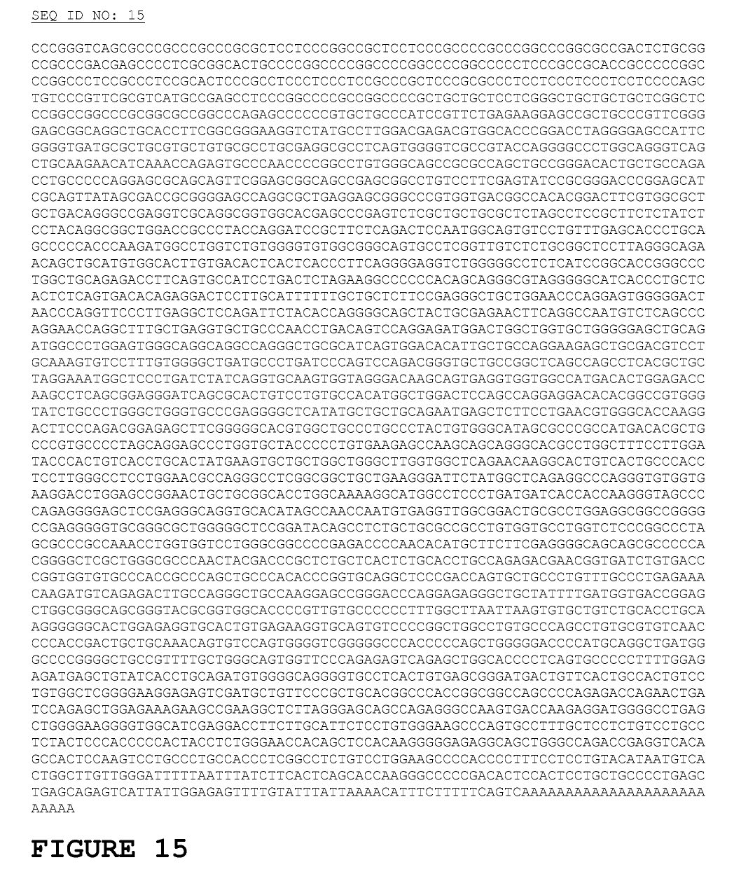

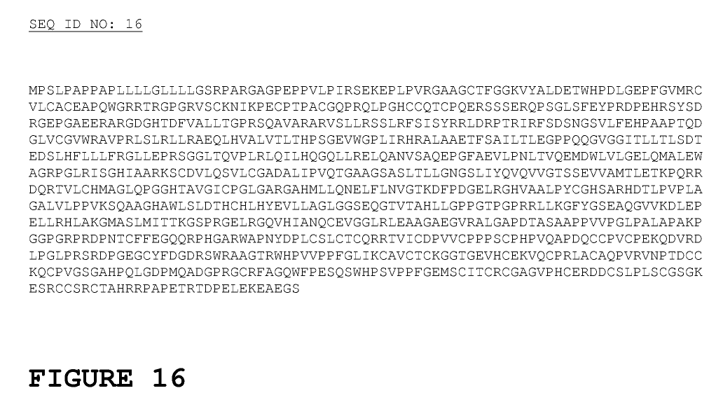

In another preferred embodiment, the BMP-inhibitor is Chordin. Amino acid and nucleotide sequences for Chordin have been reported for a variety of species, including human, chimpanzee, dog, mouse, rat, and chicken. In preferred embodiments, Chordin is a mammalian Chordin. In particularly preferred embodiments, Chordin is a human Chordin (hChordin). Exemplary nucleotide and amino acid sequences for human Chordin are set forth in SEQ ID NOs: 15 and 16, respectively (see FIG. 15 and FIG. 16).

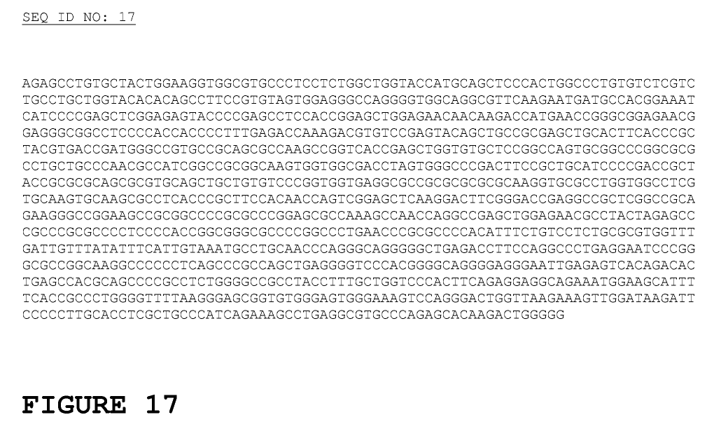

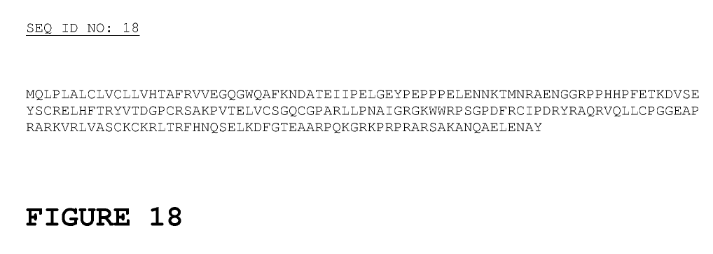

In another preferred embodiment, the BMP-inhibitor is Sclerostin. Amino acid and nucleotide sequences for Sclerostin have been reported for a variety of species, including human, dog, chimpanzee, mouse, rat, and chicken. In preferred embodiments, Sclerostin is a mammalian Sclerostin. In particularly preferred embodiments, Sclerostin is a human Sclerostin (hSclerostin). Exemplary nucleotide and amino acid sequences for human Sclerostin are set forth in SEQ ID NOs: 17 and 18, respectively (see FIG. 17 and FIG. 18).

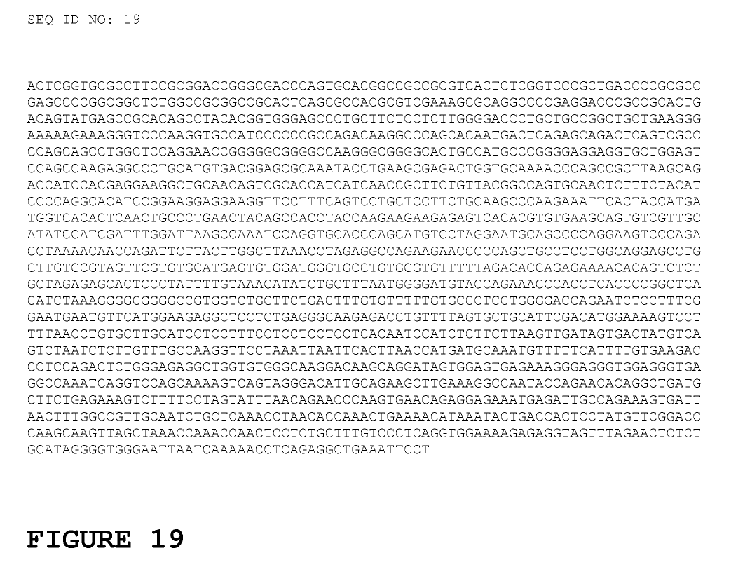

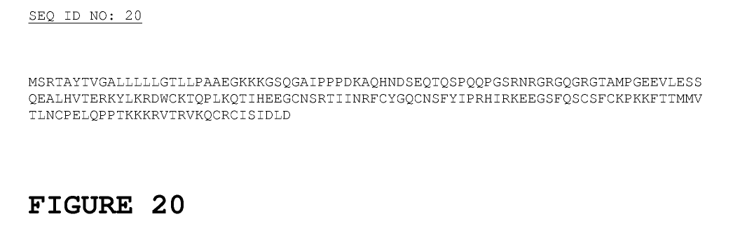

In another preferred embodiment, the BMP-inhibitor is Gremlin. Amino acid and nucleotide sequences for Gremlin have been reported for a variety of species, including human, dog, chimpanzee, mouse, rat, and chicken. In preferred embodiments, Gremlin is a mammalian Gremlin. In particularly preferred embodiments, Gremlin is a human Gremlin (hGremlin). Exemplary nucleotide and amino acid sequences for human Gremlin are set forth in SEQ ID NOs: 19 and 20, respectively (see FIG. 19 and FIG. 20).

By “recombinant bone morphogenic protein inhibitor” or “recombinant BMP-inhibitor” is meant a protein, or protein fragment thereof, with the ability to bind and/or inhibit the activity of a bone morphogenic protein (BMP) family member, produced by a transiently transfected, stably transfected, or transgenic host cell or animal as directed by one of the expression constructs of the invention. The terms “recombinant bone morphogenic protein inhibitor”, “recombinant BMP-inhibitor” also encompass recombinant BMP-inhibitor proteins in monomeric, homodimeric, heterodimeric or fused chimeric forms.

By “BMP/BMP-inhibitor complex” is meant a protein-protein association between a BMP and a BMP-inhibitor protein. The term “BMP/BMP-inhibitor complex” encompasses BMP and BMP-inhibitor protein associations in any and all monomeric, homodimeric, heterodimeric and heteromeric forms.

By “recombinant BMP/BMP-inhibitor complex” is meant a protein-protein association between a recombinant BMP and a recombinant BMP-inhibitor protein. The term “recombinant BMP/BMP-inhibitor complex” encompasses recombinant BMP and recombinant BMP-inhibitor protein associations in any and all monomeric, homodimeric, heterodimeric and heteromeric forms.

By “BMP/BMP-inhibitor fusion protein”, “BMP-inhibitor/BMP fusion protein”, “BMP/linker/BMP-inhibitor fusion protein”, or “BMP-inhibitor/linker/BMP fusion protein” is meant a chimeric protein fusion between a BMP and a BMP-inhibitor protein, containing a linker region located between the BMP and BMP-inhibitor proteins, made up of variable amino acid composition and length between, and further containing an encoded proteolytic cleavage site.

By “linker” or “linker region” is meant a peptide sequence containing amino acids with side chains of varied chemical characteristics, such as hydrophobicity, hydrophilicity, acidity and basicity, and variable length. In addition, the linker would contain an amino acid sequence encoding a proteolytic cleavage site. In a preferred embodiment, the length of the amino acid linker region would be at least 25 Angstroms.

By “genetically-engineered nucleic acid sequence” is meant a nucleic acid sequence wherein the component sequence elements of the nucleic acid sequence are organized within the nucleic acid sequence in a manner not found in nature. Such a genetically-engineered nucleic acid sequence may be found, for example, ex vivo as isolated DNA, in vivo as extra-chromosomal DNA, or in vivo as part of the genomic DNA. It is contemplated that the nucleic acid is isolated from its natural source.

By “expression construct” or “construct” is meant a nucleic acid sequence comprising a target nucleic acid sequence or sequences whose expression is desired, operably linked to sequence elements which provide for the proper transcription and translation of the target nucleic acid sequence(s) within the chosen host cells. Such sequence elements may include a promoter, a signal sequence for secretion, a polyadenylation signal, intronic sequences, insulator sequences, and other elements described in the invention. The “expression construct” or “construct” may further comprise “vector sequences.” By “vector sequences” is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

By “operably linked” is meant that a target nucleic acid sequence and one or more regulatory sequences (e.g., promoters) are physically linked so as to permit expression of the polypeptide encoded by the target nucleic acid sequence within a host cell.

By “operably linked” is further meant that two or more nucleic acid sequences, each encoding a distinct amino acid sequence, are physically linked so as to permit expression of all of the encoded polypeptide sequences as a single polypeptide within a host cell.

By “signal sequence” is meant a nucleic acid sequence which, when incorporated into a nucleic acid sequence encoding a polypeptide, directs secretion of the translated polypeptide (e.g., a BMP protein) from cells which express said polypeptide. The signal sequence is preferably located at the 5′ end of the nucleic acid sequence encoding the polypeptide, such that the polypeptide sequence encoded by the signal sequence is located at the N-terminus of the translated polypeptide. By “signal peptide” is meant the peptide sequence resulting from translation of a signal sequence.

As used herein, the term “polypeptide” or “protein” refers to a polymer of amino acid monomers that are alpha amino acids joined together through amide bonds. Polypeptides are therefore at least two amino acid residues in length, and are usually longer. Generally, the term “peptide” refers to a polypeptide that is only a few amino acid residues in length. A polypeptide, in contrast with a peptide, may comprise any number of amino acid residues. Hence, the term polypeptide included peptides as well as longer sequences of amino acids.

By “mammary gland-specific promoter” is meant a promoter that drives expression of a polypeptide encoded by a nucleic acid sequence to which the promoter is operably linked, where said expression occurs primarily in the in the mammary cells of the mammal, wherefrom the expressed polypeptide may be secreted into the milk. Preferred mammary gland-specific promoters include the β-casein promoter and the whey acidic protein (WAP) promoter.

By “host cell” is meant a cell which has been transfected with one or more expression constructs of the invention. Such host cells include mammalian cells in in vitro culture and cells found in vivo in an animal. Preferred in vitro cultured mammalian host cells include primary fetal goat cells, embryonic stem cells, embryonal carcinoma cells, primordial germ cells, AND mammary epithelium cell lines.

By “transfection” is meant the process of introducing one or more of the expression constructs of the invention into a host cell by any of the methods well established in the art, including (but not limited to) microinjection, electroporation, liposome-mediated transfection, calcium phosphate-mediated transfection, or virus-mediated transfection. A host cell into which an expression construct of the invention has been introduced by transfection is “transfected”. By “transiently transfected cell” is meant a host cell wherein the introduced expression construct is not permanently integrated into the genome of the host cell or its progeny, and therefore may be eliminated from the host cell or its progeny over time. By “stably transfected cell” is meant a host cell wherein the introduced expression construct has integrated into the genome of the host cell and its progeny.

By “transgene” is meant any segment of an expression construct of the invention which has become integrated into the genome of a transfected host cell. Host cells containing such transgenes are “transgenic.” Animals composed partially or entirely of such transgenic host cells are “transgenic animals.” Preferably, the transgenic animals are transgenic mammals (e.g., rodents or ruminants). Animals composed partially, but not entirely, of such transgenic host cells are “chimeras” or “chimeric animals”.

Assembly of Expression Constructs

The recombinant DNA methods employed in practicing the present invention are standard procedures, well-known to those skilled in the art (as described, for example, Glover and Hames, eds. DNA Cloning: A Practical Approach Vol I. Oxford University Press, 1995; Glover and Hames, eds. DNA Cloning: A Practical Approach Vol II. Oxford University Press, 1995; Glover and Hames, eds. DNA Cloning: A Practical Approach Vol III. Oxford University Press, 1996; Glover and Hames, eds. DNA Cloning: A Practical Approach, Vol IV. Oxford University Press, 1996; Gait, ed. Oligonucleotide Synthesis. 1984; Hames and Higgens, eds. Nucleic Acid Hybridization. 1985; Hames and Higgens, eds. Transcription and Translation. 1984; Perbal, A Practical Guide to Molecular Cloning. 1984; Ausubel et al., eds. Current Protocols in Molecular Biology. John Wiley & Sons, Inc. 1994; Sambrook et al. Molecular Cloning: A Laboratory Manual, Third Edition. Cold Spring Harbor Laboratory Press. 2001; Dieffenbach and Dveksler, eds. PCR Primer: A Laboratory Manual, Second Edition. Cold Spring Harbor Laboratory Press. 2003; and Ashley, ed. PCR 2: A Practical Approach. Oxford University Press. 1996). These standard molecular biology techniques can be used to prepare the expression constructs of the invention.

The expression constructs of the invention comprise elements necessary for proper transcription and translation of a target BMP-encoding nucleic acid sequence within the chosen host cells, including a promoter, a signal sequence to provide secretion of the translated product, and a polyadenylation signal. Such expression constructs may also contain intronic sequences or untranslated cDNA sequences intended to improve transcription efficiency, translation efficiency, and/or mRNA stability. The BMP-encoding nucleic acid sequence intended for expression may possess its endogenous 3′ untranslated sequence and/or polyadenylation signal or contain an exogenous 3′ untranslated sequence and/or polyadenylation signal. For example the promoter, signal sequence, and 3′ untranslated sequence and polyadenylation signal of casein may be used to mediate expression of a nucleic acid sequence encoding a BMP within mammary host cells. Codon selection, where the target nucleic acid sequence of the construct is engineered or chosen so as to contain codons preferentially used within the desired host cell, may be used to minimize premature translation termination and thereby maximize expression.

The expression constructs of the invention which provide expression of a BMP protein in the desired host cells may include one or more of the following basic components.

A) Promoter

These sequences may be endogenous or heterologous to the host cell to be modified, and may provide ubiquitous (i.e., expression occurs in the absence of an apparent external stimulus and is not cell-type specific) or tissue-specific (also known as cell-type specific) expression. Promoter sequences for ubiquitous expression may include synthetic and natural viral sequences [e.g., human cytomegalovirus immediate early promoter (CMV); simian virus 40 early promoter (SV40); Rous sarcoma virus (RSV); or adenovirus major late promoter] which confer a strong level of transcription of the nucleic acid molecule to which they are operably linked. The promoter can also be modified by the deletion and/or addition of sequences, such as enhancers (e.g., a CMV, SV40, or RSV enhancer), or tandem repeats of such sequences. The addition of strong enhancer elements may increase transcription by 10-100 fold.

For specific expression in the mammary tissue of transgenic animals, the promoter sequences may be derived from a mammalian mammary-specific gene. Examples of suitable mammary-specific promoters include: the whey acidic protein (WAP) promoter (see, e.g., U.S. Pat. Nos. 5,831,141 and 6,268,545; Andres et al. Proc Natl Acad Sci USA 1987; 84:1299-1303; and Velander et al. Proc Natl Acad Sci USA 1992; 89:12003-12007), αS1-casein (see, e.g., U.S. Pat. Nos. 4,873,316, 5,750,172, and 6,013,857; and PCT Publication Nos. WO91/08216 and WO93/25567), αS2-casein, β-casein (see, e.g., U.S. Pat. No. 5,304,489 and Lee, et al. Nucleic Acids Res. 1988; 16:1027-1041), κ-casein (see, e.g., Baranyi et al. Gene 1996; 174:27-34 and Gutierrez et al. Transgenic Research 1996; 5:271-279), β-lactoglobin (see, e.g., McClenaghan et al. Biochem. J. 1995; 310:637-641), α-lactalbumin (see, e.g., Vilotte et al. Eur. J. Biochem. 1989; 186:43-48 and PCT Publication No. WO88/01648), and the long terminal repeat (LTR) promoter of the mouse mammary tumor virus (saee, e.g., Romagnolo et al. Mol Cell Endocrinol. 1993; 96:147-157; Chaudhry et al. J Biol Chem. 1999; 274:7072-7081; and Ahmed et al. Cancer Res. 2002; 62:7166-7169).

B) BMP-Encoding Nucleic Acid Sequence

Suitable BMP-encoding sequences include any nucleic acid sequences that encode a BMP, including nucleic acid sequences encoding BMP-2, BMP-3, BMP-3b, BMP-4, BMP-5, BMP-6, BMP-7, BMP-8, BMP-9, BMP-10, BMP-11, BMP-12, BMP-13, BMP-14, and BMP-15, as well as nucleic acid sequences encoding allelic variants of BMPs, function conservative variants of BMPs, and mutant BMPs that retain BMP activity.

Nucleic acid sequences that encode BMP orthologs from a variety of species (including human, mouse, rat, cow, rabbit, dog, chicken, turtle, tilapia, zebrafish and Xenopus) are known in the art. For example nucleic acid sequences that encode a human BMP-2 (see, for example, Wozney et al. Science 1988; 242:1528-1534), BMP-3 (see, for example, Wozney et al. Science 1988; 242:1528-1534), BMP-3b (see, for example, Hino et al. Biochem. Biophys. Res. Commun. 1996; 223:304-310), BMP-4 (see, for example, Oida et al. DNA Seq. 1995; 5:273-275), BMP-5 (see, for example, Celeste et al. Proc Natl Acad Sci USA 1990; 87:9843-9847), BMP-6 (see, for example, Celeste et al. Proc Natl Acad Sci USA 1990; 87:9843-9847), BMP-7 (see, for example, Celeste et al. Proc Natl Acad Sci USA 1990; 87:9843-9847), BMP-8 (see, for example, Ozkaynak J. Biol. Chem. 1992; 267:25220-25227), BMP-9 (see, for example, Strausberg et al. Proc Natl Acad Sci USA 2002; 99:16899-16903), BMP-10 (see, for example, Neuhaus et al. Mech. Dev. 1999; 80:181-184); BMP-11 (see, for example, Gonzalez-Cadavid et al. Proc Natl Acad Sci USA 1998; 95:14938-14943); BMP-12 (see, for example, U.S. Pat. No. 5,658,882), BMP-13 (see, for example, U.S. Pat. No. 5,658,882), BMP-14 (see, for example, Chang et al. J. Biol. Chem. 1994; 269:28227-28234), or BMP-15 (see, for example, Dube et al Mol. Endocrinol. 1998; 12:1809-1817) have been reported.

In preferred embodiments, the nucleic acid sequence encodes BMP-2, BMP-4, BMP-6, BMP-7, or BMP-9. In particularly preferred embodiments the nucleic acid sequence encodes BMP-2, BMP-4 or BMP-7.

In preferred embodiments the nucleic acid sequence encodes a mammalian BMP (e.g., mammalian BMP-2 or mammalian BMP-7). In particularly preferred embodiments, the nucleic acid sequence encodes a human BMP (hBMP) (e.g. hBMP-2 or hBMP-7).

Nucleic acids sequences that encode a BMP-2 have been reported for a variety of species, including human, mouse, rat, rabbit, dog, chicken, turtle, zebrafish and Xenopus. In preferred embodiments, the nucleic acid sequence encodes a mammalian BMP-2. In particularly preferred embodiments, the nucleic acid sequence encodes a human BMP-2 (hBMP-2). An exemplary nucleic acid sequence that encodes a human BMP-2 is set forth in SEQ ID NO: 1. Nucleic acid sequences encoding a bovine BMP-2 are publicly available, for example, from the ATCC (ATCC Number 40310). Nucleic acid sequences encoding a human BMP-2 are publicly available, for example, from the ATCC (ATCC Number 40345).

Nucleic acids sequences that encode a BMP-7 have been reported for a variety of species, including human, mouse, rat, pig, chicken, Xenopus, and zebrafish. In preferred embodiments, the nucleic acid sequence encodes a mammalian BMP-7. In particularly preferred embodiments, the nucleic acid sequence encodes a human BMP-7 (hBMP-7). An exemplary nucleic acid sequence that encodes a human BMP-7 is set forth in SEQ ID NO: 3. Nucleic acid sequences encoding a human BMP-7 are publicly available, for example, from the ATCC (ATCC Number 68182 and ATCC Number 68020).

Nucleic acid sequences that encode a BMP-4 have been reported for a variety of species, including human, cow, sheep, dog, rat, rabbit, mouse, chicken, Xenopus, and zebrafish. In preferred embodiments, the nucleic acid sequence encodes a mammalian BMP-4. In particularly preferred embodiments, the nucleic acid sequence encodes a human BMP-4 (hBMP-4). An exemplary nucleic acid sequence that encodes human BMP-4 is set forth in SEQ ID NO: 5. Nucleic acid sequences encoding a human BMP-4 are publicly available, for example, from the ATCC (ATCC Number MGC-21303 and ATCC Number 40342).

For example, nucleic acid sequences encoding a human BMP-3 are publicly available from the ATCC (ATCC Number 558527). For example, nucleic acid sequences encoding a human BMP-6 are publicly available from the ATCC (ATCC Number 68245 and ATCC Number 68021). For example, nucleic acid sequences encoding a human BMP-8 are publicly available from the ATCC (ATCC Number 3384435).

In certain embodiments, the BMP-encoding nucleic acid sequence contains sequences that code for the signal peptide, the pro-domain, and the mature polypeptide domain of the BMP. In preferred embodiments, the BMP-encoding nucleic acid sequence contains sequences that code for the pro-domain and the mature polypeptide domain of the BMP.

The BMP-encoding nucleic acid sequence may also encode an epitope tag for easy identification and purification of the encoded polypeptide. Preferred epitope tags include myc, His, and FLAG epitope tags. The encoded epitope tag may include recognition sites for site-specific proteolysis or chemical agent cleavage to facilitate removal of the epitope tag following protein purification. For example a thrombin cleavage site could be incorporated between a recombinant BMP and its epitope tag. Epitope tags may be fused to the N-terminal end or the C-terminal end of a recombinant BMP.

C) Intron Inclusion

Nucleic acid sequences containing intronic sequences (e.g., genomic sequences) may be expressed at higher levels than intron-less sequences. Hence, inclusion of intronic sequences between the transcription initiation site and the translational start codon, 3′ to the translational stop codon, or inside the coding region of the BMP-encoding nucleic acid sequence may result in a higher level of expression.

Such intronic sequences include a 5′ splice site (donor site) and a 3′ splice site (acceptor site), separated by at least 100 base pairs of non-coding sequence. These intronic sequences may be derived from the genomic sequence of the gene whose promoter is being used to drive BMP expression, from a BMP gene, or another suitable gene. Such intronic sequences should be chosen so as to minimize the presence of repetitive sequences within the expression construct, as such repetitive sequences may encourage recombination and thereby promote instability of the construct. Preferably, these introns can be positioned within the BMP-encoding nucleic acid sequence so as to approximate the intron/exon structure of an endogenous human BMP gene.

D) Signal Sequences

Each expression construct will comprise a signal sequence to provide secretion of the translated recombinant BMP from the host cells of interest (e.g., mammary cells). Such signal sequences are naturally present in genes whose protein products are normally secreted. The signal sequences to be employed in the invention may be derived from a BMP-encoding nucleic acid sequence (e.g., a BMP gene), from a gene specifically expressed in the host cell of interest (e.g., casein gene), or from another gene whose protein product is known to be secreted (e.g., from human alkaline phosphatase, mellitin, the immunoglobulin light chain protein Igκ, or CD33); or may be synthetically derived.

E) Termination Region

Each expression construct will comprise a nucleic acid sequence which contains a transcription termination and polyadenylation sequence. Such sequences will be linked to the 3′ end of the BMP-encoding nucleic acid sequence. For example, these sequences may be derived from a BMP-encoding nucleic acid sequence (e.g., a BMP gene); may comprise the 3′ end and polyadenylation signal from the gene whose 5′-promoter region is driving BMP expression (e.g., the 3′ end of the goat β-casein gene); or may be derived from genes in which the sequences have been shown to regulate post-transcriptional mRNA stability (e.g., those derived from the bovine growth hormone gene, the β-globin genes, or the SV40 early region).

F) Other Features of the Expression Constructs

The BMP-encoding nucleic acid sequences of interest may be modified in their 5′ or 3′ untranslated regions (UTRs) and/or in regions coding for the N-terminus of the BMP enzyme so as to preferentially improve expression. Sequences within the BMP-encoding nucleic acid sequence may be deleted or mutated so as to increase secretion and/or avoid retention of the recombinant BMP within the cell, as regulated, for example, by the presence of endoplasmic reticulum retention signals or other sorting inhibitory signals.

In addition, the expression constructs may contain appropriate sequences located 5′ and/or 3′ of the BMP-encoding nucleic acid sequences that will provide enhanced integration rates in transduced host cells (e.g., ITR sequences as per Lebkowski et al. Mol. Cell. Biol. 1988; 8:3988-3996). Furthermore, the expression construct may contain nucleic acid sequences that possess chromatin opening or insulator activity and thereby confer reproducible activation of tissue-specific expression of a linked transgene. Such sequences include Matrix Attachment Regions (MARs) (McKnight et al. Mol Reprod Dev 1996; 44:179-184 and McKnight et al. Proc Natl Acad Sci USA 1992; 89:6943-6947). See also Ellis et al., PCT publication No.: WO95/33841 and Chung and Felsenfield, PCT Publication No.: WO96/04390.

The expression constructs further comprise vector sequences which facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

The expression constructs used for the generation of transgenic animals may be linearized by restriction endonuclease digestion prior to introduction into a host cell. In a variant of this method, the vector sequences are removed prior to introduction into host cells, such that the introduced linearized fragment is comprised solely of the BMP-encoding sequence, 5′-end regulatory sequences (e.g., the promoter), and 3′-end regulatory sequences (e.g., the 3′ transcription termination and polyadenylation sequences), and any flanking insulators or MARs. A cell transformed with such a fragment will not contain, for example, an E. coli origin of replication or a nucleic acid molecule encoding an antibiotic-resistance protein (e.g., an ampicillin-resistance protein) used for selection of transformed prokaryotic cells.

In another variant of this method, the restriction digested expression construct fragment used to transfect a host cell will include a BMP-encoding sequence, 5′ and 3′ regulatory sequences, and any flanking insulators or MARs, linked to a nucleic acid sequence encoding a protein capable of conferring resistance to a antibiotic useful for selection of transfected eukaryotic cells (e.g., neomycin or puromycin).

Generation of Transfected Cell Lines In Vitro

The expression constructs of the invention may be transfected into host cells in vitro. Preferred in vitro host cells are mammalian cell lines including primary fetal goat cells, R1 embryonic stem cells, embryonal carcinoma cells, primordial germ cells, and mammary epithelium cell lines [e.g., human mammary epithelium cell lines 184B5 (ATCC Number CRL-8799), 184A1 (ATCC Number CRL-8798), MCF7 (ATCC Number HTB-22), and ZR-75-30 (ATCC Number CRL-1504) or the bovine mammary epithelium cell line MAC-T cell (ATCC Number CRL 10274)].

Protocols for in vitro culture of mammalian cells are well established in the art (see for example, Masters, ed. Animal Cell Culture: A Practical Approach 3^{rd} Edition. Oxford University Press, 2000 and Davis, ed. Basic Cell Culture, 2^{nd} Edition. Oxford University Press, 2002).

Techniques for transfection are well established in the art and may include electroporation, microinjection, liposome-mediated transfection, calcium phosphate-mediated transfection, or virus-mediated transfection (see, for example, Felgner, ed. Artificial self-assembling systems for gene delivery. Oxford University Press, 1996; Lebkowski et al. Mol. Cell Biol. 1988; 8:3988-3996; Ausubel et al., eds. Current Protocols in Molecular Biology. John Wiley & Sons, Inc., 1994; and Sambrook et al. Molecular Cloning: A Laboratory Manual, Third Edition. Cold Spring Harbor Laboratory Press, 2001). Where stable transfection of the host cell lines is desired, the introduced DNA preferably comprises linear expression construct DNA, free of vector sequences, as prepared from the expression constructs of the invention. Transfected in vitro cell lines may be screened for integration and copy number of the expression construct. For such screening, the genomic DNA of a cell line is prepared and analyzed by PCR and/or Southern blot.

Transiently and stably transfected cell lines may be used to evaluate the expression constructs of the invention as detailed below, and to isolate recombinant BMP protein. Where the expression construct comprises a ubiquitous promoter any of a number of established mammalian cell culture lines may be transfected. Where the expression construct comprises a tissue-specific promoter, the host cell line should be compatible with the tissue specific promoter. Typically the immortalized cell line MAC-T, established from bovine mammary epithelia cells are used to evaluate the suitability of mammary specific expression vectors (see, e.g., Huynh et al. Exp Cell Res. 1991; 197:191-199).

Stably transfected cell lines may also be used to generate transgenic animals. For this use, the recombinant proteins need not be expressed in the in vitro cell line.

Evaluation of Expression Constructs

Prior to the generation of transgenic animals using the expression constructs of the invention, expression construct functionality can be determined using transfected in vitro cell culture systems. Genetic stability of the expression constructs, degree of secretion of the recombinant protein(s), and physical and functional attributes of the recombinant protein(s) can be evaluated prior to the generation of transgenic animals.

Where the expression construct comprises a ubiquitous promoter any of a number of established mammalian cell culture lines may be transfected. Where the expression construct comprises a mammary gland-specific promoter, mammary epithelium cell lines can be transfected [e.g., the bovine mammary epithelium cell line MAC-T (ATCC Number CRL 10274) or the human mammary epithelium cell lines 184B5 (ATCC Number CRL-8799), 184A1 (ATCC Number CRL-8798), MCF7 (ATCC Number HTB-22), or ZR-75-30 (ATCC Number CRL-1504)].

To determine if cell lines transfected with the BMP-encoding expression constructs of the invention are producing recombinant BMP, the media from transfected cell cultures can be tested directly for the presence of a secreted BMP protein (see the section Assays to characterize BMP, below). The characteristics and activity of the recombinant BMP may be assessed by any of the methods well established in the art (see the section Assays to characterize BMP, below).

Generation of Transgenic Mammals

Protocols for the generation of non-human transgenic mammals are well established in the art (see, for example, Murphy et al., eds. Transgenesis Techniques. Human Press, Totowa, N.J., 1993; Puhler, ed. Genetic Engineering of Animals. VCH Verlagsgesellschaft, Weinheim, N.Y., 1993; Murray et al., eds. Transgenic Animals in Agriculture. Oxford University Press, 1999; and Jackson and Abbott, eds. Mouse Genetics and Transgenics: A Practical Approach. Oxford University Press, 2000). For example, efficient protocols are available for the production of transgenic mice (see, for example, Hogan et al. Manipulating the Mouse Embryo 2^{nd} Edition. Cold Spring Harbor Press, 1994 and Mouse Genetics and Transgenics: A Practical Approach. Oxford University Press, 2000), transgenic cows (see, for example, U.S Pat. No. 5,633,076), transgenic pigs (see, for example, U.S. Pat. No. 6,271,436), transgenic sheep (see, for example, U.S. Pat. No. 4,873,316), and transgenic goats (see, for example, U.S. Pat. No. 5,907,080 and Keefer et al. Biol Reprod 2001; 64:849-856). Preferred examples of such protocols are summarized below. It will be appreciated that these examples are not intended to be limiting, and that transgenic non-human mammals comprising the expression constructs of the invention, as created by these or other protocols, necessarily fall within the scope of the invention.

For example, transgenic animals may be generated using stably transfected host cells derived from in vitro transfection. Where said host cells are pluripotent or totipotent, such cells may be used in morula aggregation or blastocyst injection protocols to generate chimeric animals. Preferred pluripotent/totipotent stably transfected host cells include primordial germ cells, embryonic stem cells, and embryonal carcinoma cells. In a morula aggregation protocol, stably transfected host cells are aggregated with non-transgenic morula-stage embryos. In a blastocyst injection protocol, stably transfected host cells are introduced into the blastocoelic cavity of a non-transgenic blastocyst-stage embryo. The aggregated or injected embryos are then transferred to a pseudopregnant recipient female for gestation and birth of chimeras. Chimeric animals in which the transgenic host cells have contributed to the germ line may be used in breeding schemes to generate non-chimeric offspring which are wholly transgenic.

In an alternative protocol, such stably transfected host cells may be used as nucleus donors for nuclear transfer into recipient oocytes (see, for example, U.S. Pat. No. 6,147,276 and Wilmut et al. Nature 1997; 385:810-813). For nuclear transfer, the stably transfected host cells need not be pluripotent or totipotent. Thus, for example, stably transfected fetal fibroblasts can be used (see, e.g., Cibelli et al. Science 1998; 280:1256-1258 and Keefer et al. Biol Reprod 2001; 64:849-856). The recipient oocytes are preferably enucleated prior to transfer. Following nuclear transfer, the oocyte is transferred to a pseudopregnant recipient female for gestation and birth. Such offspring will be wholly transgenic (that is, not chimeric).

In another alternative protocol, transgenic animals are generated by direct introduction of expression construct DNA into a recipient oocyte, zygote, or embryo. Such direct introduction may be achieved, for example, by pronuclear microinjection (see, e.g., Wang et al. Mol Reprod Dev 2002; 63:437-443), cytoplasmic microinjection (see, e.g., Page et al. Transgenic Res 1995; 4:353-360), retroviral infection (see, e.g., Lebkowski et al. Mol. Cell Biol 1988; 8:3988-3996), or electroporation (see, e.g., Sambrook et al. Molecular Cloning: A Laboratory Manual, Third Edition. Cold Spring Harbor Laboratory Press, 2001).

For microinjection and electroporation protocols, the introduced DNA should comprise linear expression construct DNA, free of vector sequences, as prepared from the expression constructs of the invention. Following DNA introduction and any necessary in vitro culture, the oocyte, zygote, or embryo is transferred to a pseudopregnant recipient female for gestation and birth. Such offspring may or may not be chimeric, depending on the timing and efficiency of transgene integration. For example, if a single cell of a two-cell stage embryo is microinjected, the resultant animal will most likely be chimeric.

Transgenic animals comprising two or more independent transgenes can be made by introducing two or more different expression constructs into host cells using any of the above described methods.

The presence of the transgene in the genomic DNA of an animal, tissue, or cell of interest, as well as transgene copy number, may be confirmed by techniques well known in the art, including hybridization and PCR techniques.

Some of the transgenic protocols result in the production of chimeric animals. Chimeric animals in which the transgenic host cells have contributed to the tissue-type wherein the promoter of the expression construct is active (e.g., mammary gland for WAP promoter) may be used to characterize or isolate recombinant BMP protein. More preferably, where the transgenic host cells have contributed to the germ line, chimeras may be used in breeding schemes to generate non-chimeric offspring which are wholly transgenic.

Wholly transgenic offspring, whether generated directly by a transgenic protocol or by breeding of a chimeric animal, may be used for breeding purposes to maintain the transgenic line and to characterize or isolate recombinant BMP protein. Where transgene expression is driven by a mammary gland-specific promoter, lactation of the transgenic animals may be induced or maintained, where the resultant milk may be collected for purification and characterization of recombinant BMP protein. For female transgenics, lactation may be induced by pregnancy or by administration of hormones. For male transgenics, lactation may be induced by administration of hormones (see, for example, Ebert et al. Biotechnology 1994; 12:699-702). Lactation is maintained by continued collection of milk from a lactating transgenic.

Purification of Recombinant BMP

Recombinant BMP may be purified from transgenic animals expressing recombinant BMP in mammary gland according to any of the techniques well established in the art, including affinity separation, chromatography, and immunoprecipitation. Such techniques are well described in the art (see, for example, such methods are well known in the art (See for example, Ausubel et al., eds. Current Protocols in Molecular Biology. John Wiley & Sons, Inc. 1994; Coligan et al., eds. Current Protocols in Immunology. John Wiley & Sons, Inc. 1991; Sambrook et al. Molecular Cloning: A Laboratory Manual, Third Edition. Cold Spring Harbor Laboratory Press. 2001; Harlow and Lane. Using Antibodies: A Laboratory Manual. Cold Spring Harbor Laboratory Press. 1999; Gosling, ed. Immunoassays: A Practical Approach. Oxford University Press. 2000; Matejtschuk, ed. Affinity Separations: A Practical Approach. Oxford University Press, 1997; Oliver, ed. HPLC of Macromolecules: A Practical Approach. Oxford University Press, 1998; Millner, ed. High Resolution Chromatography: A Practical Approach. Oxford University Press, 1999; and Roe, ed. Protein Purification Techniques: A Practical Approach. Oxford University Press, 2001).

In particular, protocols for the purification of BMPs have been described (see, for example, by U.S. Pat. No. 4,761,471; U.S. Pat. No. 4,789,732; U.S. Pat. No. 4,795,804; U.S. Pat. No. 4,877,864; U.S. Pat. No. 5,013,649; U.S. Pat. No. 5,618,924; U.S. Pat. No. 5,631,142; U.S. Pat. No. 6,593,109; Wang et al. Proc Natl Acad Sci USA 1990; 87:2220-2224; Vallejo et al. J Biotech 2002; 94:185-194; Hu et al. Growth Factors 2004; 22:29-33; and Vallejo et al. Biotech Bioeng 2004; 85:601-609). In particular, protocols for the purification of BMP heterodimers, including BMP-2/-7 heterodimers and BMP-2/-6 heterodimers have been described (see, for example, U.S. Pat. No. 6,593,109 and Aono et al. Biochem Biophys Res Comm. 1995; 210:670-677)

In preferred embodiments, recombinant BMP is purified by heparin affinity chromatography. BMP dimers have greater affinity for heparin than do BMP homodimers, thus by using heparin affinity chromatography for purification of recombinant BMP, the active dimer is selectively purified. Techniques for the purification of BMP by heparin affinity chromatography are well known in the art (see, for example, U.S. Pat. No. 5,013,649; U.S. Pat. No. 5,166,058; U.S. Pat. No. 5,631,142; Wang et al. Proc Natl Acad Sci USA 1990; 87:2220-2224; and Vallejo et al. J Biotech 2002; 94:185-194).

The recombinant BMP may be purified, for example, from mammary gland tissue collected from a transgenic animal expressing recombinant BMP in mammary gland, or from milk collected from a transgenic animal expressing recombinant BMP in mammary gland. In preferred embodiments, recombinant BMP is purified from milk collected from a transgenic animal expressing recombinant BMP in mammary gland.

In particularly preferred embodiments, recombinant BMP is purified from milk collected from a transgenic animal expressing BMP in the mammary gland by heparin affinity chromatography.

Assays to Characterize BMP

Various assays may be used to characterize the recombinant BMP expressed by transiently or stably transfected host cells, or by transgenic animals expressing recombinant BMP in the mammary gland. The recombinant BMP so characterized may be, for example, the recombinant BMP secreted into the culture medium of a stably or transiently transfected host cell, the recombinant BMP as found in vivo in the mammary gland of the transgenic animal, the recombinant BMP as found in milk collected from the transgenic animal, or the recombinant BMP as purified from the milk of the transgenic animal. Suitable assays include, for example, assays to characterize protein levels, protein purity, activity, stability, structural characteristics, and in vitro and in vivo function of recombinant BMPs.

For example, the amount of recombinant BMP protein produced may be quantitated by any of the techniques well known in the art, including denaturing or non-denaturing gel electrophoresis, Western blotting, immunoassay (e.g., enzyme linked immunosorbent assays, ELISA), immunohistochemistry, electrometry, spectrophotometry, chromatography (e.g., high pressure liquid chromatography, HPLC and ion-exchange chromatography) and radiometric methodologies. In addition, various physical characteristics of the recombinant BMP may be characterized, including primary amino acid sequence, protein purity, molecular weight, isoelectric point, subunit composition (e.g., monomeric, homodimeric, heterodimeric), glycosylation profile, by any of the techniques well known in the art, including denaturing or non-denaturing gel electrophoresis, Western blotting, immunoassay (e.g., enzyme linked immunosorbent assays, ELISA), immunohistochemistry, electrometry, spectrophotometry, chromatography (e.g., high pressure liquid chromatography, HPLC and ion-exchange chromatography) and radiometric methodologies.

Such methods are well known in the art (see, for example, such methods are well known in the art (See for example, Ausubel et al., eds. Current Protocols in Molecular Biology. John Wiley & Sons, Inc. 1994; Coligan et al., eds. Current Protocols in Immunology. John Wiley & Sons, Inc. 1991; Sambrook et al. Molecular Cloning: A Laboratory Manual, Third Edition. Cold Spring Harbor Laboratory Press. 2001; Harlow and Lane. Using Antibodies: A Laboratory Manual. Cold Spring Harbor Laboratory Press. 1999; Gosling, ed. Immunoassays: A Practical Approach. Oxford University Press. 2000; Matejtschuk, ed. Affinity Separations: A Practical Approach. Oxford University Press, 1997; Oliver, ed. HPLC of Macromolecules: A Practical Approach. Oxford University Press, 1998; Millner, ed. High Resolution Chromatography: A Practical Approach. Oxford University Press, 1999; Roe, ed. Protein Purification Techniques: A Practical Approach. Oxford University Press, 2001; Hockfield et al. Selected Methods for Antibody and Nucleic Acid Probes. Cold Spring Harbor Laboratory Press. 1993; Gore, ed. Spectrophotometry and Spectrofluorimetry: A Practical Approach. Oxford University Press, 2000'; and Higgins and Hames, eds. Post-Translational Processing: A Practical Approach. Oxford University Press, 1999).

In particular, protocols for the characterization of BMP proteins by protein concentration determination, tryptic peptide mapping, amino acid content analysis, amino acid sequence determination, molecular weight determination, isoelectric point determination, N-terminal sequence analysis, and characterization of subunit composition (e.g., monomer versus dimer) have been described (see, for example, U.S. Pat. No. 4,761,471; U.S. Pat. No. 4,789,732; U.S. Pat. No. 4,795,804; U.S. Pat. No. 4,877,864; U.S. Pat. No. 5,013,649; U.S. Pat. No. 5,166,058; U.S. Pat. No. 5,618,924; U.S. Pat. No. 5,631,142; Wang et al. Proc Natl Acad Sci USA 1990; 87:2220-2224; and Vallejo et al. J Biotech 2002; 94:185-194).

For example, recombinant BMP may be separated on Sephacryl S-300 to distinguish the monomeric, homodimeric, and heterodimeric forms of the protein. For example, the primary amino acid sequence, and in particular the sequence of the amino terminus, of recombinant BMP may be determined by protein sequencing.

For example, protocols for radioimmunoassay analysis of BMP proteins have been described (see, for example, U.S. Pat. No. 4,857,456). For example, protocols for immunoblot analysis of BMP proteins have been described (see, for example, Wang et al. Proc Natl Acad Sci USA 1990; 87:2220-2224). For example, ELISA kits for the quantitation of protein levels of human, rat, or mouse BMP-2 are commercially available, for example, from R&D Systems (catalog #DBP200, PDBP200, or SBP200). For example, ELISA kits for the quantitation of protein levels of human BMP-7 are commercially available, for example, from R&D Systems (catalog #DY354 or DY354E). For example, a panel of monoclonal antibodies may be used to characterize the functional domains of the recombinant BMP. A variety of polyclonal and monoclonal antibodies for the various BMPs are available from a variety of commercial sources, including Chemicon, Alpha Diagnostics International, Novus Biologicals, Abcam, Abgent, and Calbiochem.

Assays to characterize in vitro and in vivo function of recombinant BMPs are well known in the art, (see, e.g., U.S. Pat. No. 4,761,471; U.S. Pat. No. 4,789,732;.U.S. Pat. No. 4,795,804; U.S. Pat. No. 4,877,864; U.S. Pat. No. 5,013,649; U.S. Pat. No. 5,166,058; U.S. Pat. No. 5,618,924; U.S. Pat. No. 5,631,142; U.S. Pat. No 6,150,328; U.S. Pat. No. 6,593,109; Clokie and Urist Plast. Reconstr. Surg. 2000; 105:628-637; Kirsch et al. EMBO J 2000; 19:3314-3324; Vallejo et al. J Biotech 2002; 94:185-194; Peel et al. J Craniofacial Surg. 2003; 14:284-291; and Hu et al. Growth Factors 2004; 22:29-33;

Such assays include: in vivo assays to quantitate oseoinductive activity of a BMP following implantation (e.g., into hindquarter muscle or thoracic area) into a rodent (e.g. a rat or a mouse) (see, for example, U.S. Pat. No. 4,761,471; U.S. Pat. No. 4,789,732; U.S. Pat. No. 4,795,804; U.S. Pat. No. 4,877,864; U.S. Pat. No. 5,013,649; U.S. Pat. No. 5,166,058; U. S. Pat. No. 5,618,924; U.S. Pat. No. 5,631,142; U.S. Pat. No 6,150,328; U.S. Pat. No. 6,503,109; Kawai and Urist. Clin Orthop Relat Res 1988; 222:262-267; Clokie and Urist Plast. Reconstr. Surg. 2000; 105:628-637; and Hu et al. Growth Factors 2004; 22:29-33); in vivo assays to quantitate activity of a BMP to regenerate skull trephine defects in mammals (e.g., rats, dogs, or monkeys) (see, for example, U.S. Pat. No. 4,761,471 and U.S. Pat. No. 4,789,732); in vitro assays to quantitate activity of a BMP to induce proliferation of in vitro cultured cartilage cells (see, for example, U.S. Pat. No. 4,795,804); in vitro assays to quantitate activity of a BMP to induce alkaline phosphatase activity in in vitro cultured muscle cells [e.g., C2C12 cells (ATCC Number CRL-1772)] or bone marrow stromal cells [e.g., murine W-20 cells (ATCC Number CRL-2623)] (see, for example, U.S. Pat. No. 6,593,109; Ruppert et al. Eur J Biochem 1996; 237:295-302; Kirsch et al. EMBO J 2000; 19:3314-3324; Vallejo et al. J Biotech 2002; 94:185-194; Peel et al. J Craniofacial Surg. 2003; 14:284-291; and Hu et al. Growth Factors 2004; 22:29-33); in vitro assays to quantitate activity of a BMP to induce FGF-receptor 2 (FGFR3) expression in in vitro cultured mesenchymal progenitor cell lines (e.g., murine C3H10T1-2 cells) (see, for example, Vallejo et al. J Biotech 2002; 94:185-194); in vitro assays to quantitate activity of a BMP to induce proteoglycan synthesis in chicken limb bud cells (see, for example, Ruppert et al. Eur J Biochem 1996; 237:295-302); and in vitro assays to quantitate activity of a BMP to induce osteocalcin treatment in muscle cells [e.g. murine C2C12 cells (ATCC Number CRL-1772)] (see for example Katagiri et al. J Cell Biol. 1994; 127:1755-66) or bone marrow stromal cells [e.g., murine W-20 cells (ATCC Number CRL-2623)] (see, for example, U.S. Pat. No. 6,593,109).

Alternate Methods for the Production of BMP

It is possible under certain conditions that the regulatory elements which control the expression of the rhBMP genes could function within the mammary gland, other tissues of the host transgenic animal, or cells in culture, at very low levels. Despite this low level expression, the extraordinary potency of the BMPs could produce negative biological effects either in vivo, such as in the host transgenic animal or in vitro, such as in cell culture.

The regulation of casein gene expression is well documented as being tissue specific to the mammary secretary epithelial cells within mammary tissue and only occurs in the presence of lactogenic signals. In addition, these signals are present only in female mammals during late gestation and post-parturition. For these reasons the casein regulatory elements are the promoters of choice for genetic engineers wishing to over-express recombinant proteins in the mammary gland. However, in certain cases, where the recombinant protein possess very high potency, like the rhBMPs which exhibit biological activity at levels as low as 1 ng/ml, if the casein promoter “leaked” even slightly (eg. exhibit a low basal expression level), the development and function of the mammary gland could be compromised.

The following examples describe two methods for overcoming this type of problem, should it occur. The first method describes the creation and production of an inactive pro-form of BMP, which would be used as the transgene. The second method describes the co-expression of a BMP inhibitor, along with BMP in the transgene. Both of these methods allow for the subsequent activation of the recombinant BMP by post-expression processing and purification of the rhBMP.

Production of an Inactive “Proform” of BMP

The pro-domain of the TGF-β family members, including all BMPs, has several functions. It appears to be required for the folding and secretion of mature active proteins (Gray et al. Science 247:1328-1330). Further, in the case of TGF-β, continued association of the N-terminal and C-terminal domain after proteolytic cleavage renders the complex inactive or latent (Gentry et al. Biochemistry 1990; 29:6851-6857). ProBMP-4 has been reported to be biologically inactive (Cui et al. EMBO J. 1998; 17:4735-4743), although E. coli produced proBMP-2 has been reported to posses osteoinductive activity (Hillger et al. J. Biol. Chem. 2005; 280:14974-14980) and CHO cell produced rh-proBMP-9 has similar activity as mature rhBMP-9 in various in vitro assays (Brown et al. J. Biol. Chem. 2005; 280:25111-25118).

The “pro” sequences are removed from the proBMPs via proteolytic cleavage activated by either the furin, or furin-like, proteases intracellularly during protein synthesis. The furin cleavage sequence is R-X-X-R ↓ with a higher activity when the sequence is R-X-K/R-R. (Constam et al. J. Cell Biol. 1999; 144:139-149). The enzyme plasmin, which is highly expressed in milk preferentially, cleaves K ↓-X and R ↓-X and thus might also be expected to activate proBMP. This is supported by the observation that trypsin, which has similar activity to plasmin, can activate proBMP-2 (Hillger et al. J. Biol. Chem. 2005; 280:14974-14980).

The expression of recombinant proteins as fusions to proteins that serve as an affinity tag are well known in the art. Removal of the affinity tag requires the presence of a short enzymatically cleavable peptide sequence inserted between the recombinant protein and the affinity tag. Once purification has occurred, the mature recombinant protein is released from the tag by the use of specific enzymes (Waugh et al. Trends Biotechnol; 2005; 6:316-320 and Jenny et al. Protein Expr. Purif. 2003; 31:1-11) that recognize the cleavage site.

In one embodiment the RXKR furin cleavage site is mutated to one that is resistant to furin and plasmin, but sensitive to other protease enzymes. A number of specific cleavage enzymes can be used (Table 1). In another embodiment the RXXR cleavage site is deleted and the acid labile aspartyl-proline sequence inserted (Escher et al. J. Pept Res. 2004; 63:36-47). In this manner, we would enable the expression of BMP in its inactive form, which would only become activated once it was purified from the expression milieu.

TABLE 1
Enzymes used to cleave affinity tags from recombinant proteins.

| Cleavage Agent | Cleavage Site | Comment |
|---|---|---|
| Factor Xa | IEGR\hat{ }-X | Generate proteins with native N-termini, but are promiscuous |
| Enterokinase | DDDDK\hat{ }-X | so must determine whether degrade protein internally |
| acTEV | ENLYFQ\hat{ }-G | Highly specific engineered enzymes. However they both |
| PreScission | LEVLFQ\hat{ }-GP | require presence of a C-terminal residue, which is thus left |
| | | behind on the protein. TEV is somewhat amenable to the G |
| | | being replaced by other amino acids. |
| 1M acetic acid | D\hat{ }P | The aspartyl-proline sequence is highly susceptable to |
| | | hydrolysis by mild acids. This sequence does not occur |
| | | naturally in proBMP-2, 4 or 7. |

The selection of an optimal proteolytic enzyme and cleavage conditions could be assessed through expression of the mutated rhBMP protein in vitro followed by the evaluation of various standard conditions for different enzymes. The choice of enzyme and conditions are guided by results that a) produce biologically active rhBMP via cleavage of the linker, and b) do not degrade the rhBMP via cleavage of internal sites within the rhBMP.

Production of BMP with Coexpression of Inhibitor Protein

Another method for reducing the potential deleterious effects of active BMP during heterologous expression involves the co-expression of a natural BMP inhibitor within the transgene expression system. In vivo BMP signaling is subject to extracellular control through binding with various BMP-binding proteins (Table 2). These inhibitory proteins bind BMP with high affinity (see for example Yanagita Cyt. Gr. Fact. Rev. 2005; 16:309-317) and can be deployed in a number of ways. One possible embodiment involves the co-expression of a rhBMP along with an inhibitor protein so that the inhibitory protein binds the nascent rhBMP in situ, thus limiting the biological activity of the newly formed rhBMP through the formation of an inactive complex that is secreted into the milk and subsequently collected from the animal. Active rhBMP is then recovered from the inactive complex during purification with the addition of heat and/or detergents and/or chaotropic agents (see for example Brownell et al Connective Tissue Res. 1988; 17:261-275). A second possible embodiment involves expression of the rhBMP and inhibitory protein as a fusion protein, within the same expression vector, linked by a short peptidic region containing a cleavable peptide sequence. In this manner, a BMP/linker/BMP-inhibitor fusion protein with a cleavable peptide sequence in the linker region is formed, such that it is recognized and cleaved by a specific protease enzyme (cf. Table 1) or acid hydrolysis, allowing release of the active BMP followed by purification.

TABLE 2
BMP binding/inhibiting proteins.

| Protein | Reference | Comment |
|---|---|---|
| Noggin | Zimmerman et al. 1996 | Strongly binds to BMP-2 = BMP-4 > BMP-7 |
| Chordin | Blader et al. 1997 | Similar to noggin BMP-2 = BMP-4 > BMP-7 |
| DAN | Stanley et al. 1998 | Binding demonstrated to BMP-2, BMP-4, BMP-7 |
| Gremlin | Hsu et al. 1998 | Binding demonstrated to BMP-2, BMP-4, BMP-7 |
| Sclerostin | Kusu et al. 2003 | Strongly binds BMP-6, BMP-7, weakly to BMP-2, BMP-4 |
| USAG-1 | Yanigita et al. 2004 | Strongly binds to BMP-2, BMP-4, BMP-7 |
| Follistatin | Fainsod et al. 1997 | Binding demonstrated to BMP-2, BMP-4, BMP-7 |
| A2HS/fetuin | Binkert et al 1999 | Binds to BMP-2, BMP-4 and TGF-β |

For examples of BMPs and associated inhibitor proteins, see Noggin (Zimmerman et al. Cell 1996; 86:599-606), Chordin (Blader et al. Science 1997; 278:1937-1940), DAN (Stanley et al. Mech. Dev. 1998; 77:173-184) Gremlin (Hsu et al. Mol. Cell 1998; 1:673-683), Sclerostin (Kusu et al. J. Biol. Chem. 2003; 278:24113-24117), USAG-1 (Yanagita et al. Biochem. Biophys. Res. Commun. 2004; 316:490-500), Follistatin (Fainsod et al. Mech. Dev. 1997; 63:39-50), A2HS/fetuin (Binkert et al. J. Biol. Chem. 1999; 274:28514-28520). A crystal structure of the BMP/Noggin complex has also been described (Groppe et al. Nature 2002; 420:636-642, PDB ID 1M4U).

Noggin has demonstrated affinity for BMP-2 and BMP-4 and therefore would comprise a preferred embodiment for a BMP-inhibitor when either BMP-2 or BMP-4 are being expressed as as a homodimer or in a fusion protein. Sclerostin has demonstrated affinity for BMP-7 and therefore would comprise a preferred embodiment for a BMP-inhibitor when BMP-7 is being expressed as as a homodimer or in a fusion protein. Gremlin has demonstrated affinity for BMP-2, 4 and 7 and therefore would comprise a preferred embodiment when any of BMP-2, BMP-4 or BMP-7 are being expressed as a heterodimer or in a fusion protein.

In addition to the BMP-inhibitors described above, several variants and fragments thereof have been demonstarted to exhibit BMP inhibition (see for example Millet et al. Mech. Dev. 2001; 106:85-96). It is possible to envision the utility of any encodable peptide-based BMP inhibitor in either the transgenic BMP/BMP-inhibitor coexpression or fusion-protein systems, as described below. Further, it is possible to envision that additional non-natural BMP-inhibitors can be generated by random mutagenesis and/or combinatorial mutagenesis techniques such as those employed in protein design methodologies. These involve stragetgies for the creation of peptide diversity, coupled with selection technology and include, but are not limited to, genotype-phenotype techniques such as the Yeast Two-hybrid assay and phage display. BMP inhibitor discovery by solid- or solution-phase synthetic peptide combinatorial chemistry would also be possible by someone skilled in the art.

The production of inactive BMP, by coexpression with a BMP inhibitor protein, in which the BMP protein is activatable upon purification, would eliminate any unwanted effects of inappropriate expression in the mammary gland, other organs or cells. The co-expression of the BMP-inhibitor with the BMP-protein will require that both the BMP protein and BMP-inhibitor are under the control of a promoter and operably linked to a signal sequence that provides for the expression and secretion, respectively, of both proteins. In addition, the production of inactive BMP by expression of a BMP/BMP-inhibitor fusion protein, which is activatable upon protease cleavage and purification, would also eliminate any unwanted effects of inappropriate expression in the mammary gland, other organs or cells.

Kinetics of Interaction among Noggin, BMP and BMP Receptors

A mathematical model of the kinetics of interaction between the BMPs and the inhibitory proteins would be useful in evaluating the requirements for effective inhibition of BMP activity in the presence of BMP receptors. This model would help to illustrate under which conditions the rhBMP would be bound by the inhibitor and not be available to bind with, and activate, celllular BMP receptors.

A preferred embodiment of a BMP is BMP-2. A preferred embodiment of a BMP-inhibitor protein is the Noggin protein, or portions thereof. Noggin is a 32 kDa glycoprotein that is typically secreted as a homodimer. Noggin is a competitive inhibitor of BMP-2, and for Noggin to function properly as an inhibitor in this system it must be in excess of, or bind more tightly to, BMP than BMP binds its own receptor. Thus knowledge of the binding kinetics between the two sets of protein interactions, BMP/BMP-inhibitor and BMP/BMP-receptor, is important in order to evaluate the level of BMP-2 inhibition by Noggin association in our expression systems. The binding kinetics of the two competing protein-protein interactions has been described (Zimmerman et al. Cell 1996; 86:599-606).
noggin+rhBMP ⇄noggin/rhBMP complex Interaction 1
Noggin binds BMP-2 with high affinity: K_{d}=1.9×10⁻¹¹ M (19 pM). It is worth noting that the BMP proteins can display a high affinity for their natural receptors, for example follistatin binds activin with a 1.3-200 pM affinity constant (Nakamura et al. Science 1990; 247:836-838 and Schneyer et al. Endocrinology 1994; 135:667-674). Interaction 1 will occur at a low level during animal development as only small amounts of protein are expressed as the casein promoter leaks, but will occur at a high level during production when the casein promoter is firing.
BMP-2+rhBMP-receptor ⇄rhBMP/receptor⇄complex Interaction 2
BMP-2 binds to the BMP-specific receptor typically with a K_{d} between 10⁻⁹ and 10⁻¹⁰ M (1-10 nM). This interaction must be kept to a minimum at all times within a host transgenic animal or cellular production system.

If the expression of both the BMP and BMP-inhibitor protein are under the control of the same promoter, then the transgene expression system will not create an excess of BMP inhibtor relative to BMP. Therefore, favorable inhibition of the BMP is dependent solely on the relative binding affinity of the BMP for BMP-inhibitor versus BMP-receptor. As seen above, the approximate ten-fold higher affinity of Noggin for BMP-2, over BMP for the BMP-specific receptor, suggests that Noggin could effectively compete with BMP receptors for BMP inhibition. This inhibition could take place in vivo in the mammary gland and elsewhere in the growing transgenic mammal or in in vitro cell culture should it be expressed ectopically, and could thereby limit and/or eliminate the biological effects of transgenic expression of BMP protein.

One possible variation of this model would involve the production of a fusion protein consisting of the BMP and BMP-inhibitor protein connected by a selectively cleavable linker. In this manner, binding kinetics are altered due to an increase in apparent local concentration of inhibitor, and the binding affinity between protein and inhibitor can be greatly amplified, depending on the relative disposition of the two proteins in the fusion protein. In a preferred embodiment, the BMP portion would be composed of bone morphogenetic protein 2 (BMP-2) and the BMP inhibitor would be Noggin. In this embodiment, the BMP-2 protein would be bound by Noggin and be unavailable to interact with extracellular BMP receptors and influence cellular events associated with osteogenesis while being heterologously expressed. The expression of this novel fusion protein could first established within an in vitro expression system, and within the mammary gland and other organs of developing mammals. This expression-fusion system is expected to benefit from the fact that any Noggin-BMP fusion protein would be inactive within the mammary system, yet be secreted via the typical pathway for milk protein components into the milk. Secondly, once the fusion protein is recovered from the milk, the cleavage of the linker can be achieved ex vivo to produce biologically active BMP-2.

In order to construct a nucleotide encoding a novel chimeric fusion protein employing a BMP and a BMP inhibitor protein, a number of factors must be considered. These factors include, but are not limited to 1) the order of the BMP protein and the inhibitor protein, 2) the chemical nature of the amino acids comprising the linker region between the two proteins, 3) the length of the intergenic linker region, 4) the incorporation of a protease cleavage site in the linker region, 5) the location of the protease cleavage site within the linker region, and 6) selection of a protease enzyme for cleavage of the linker region.

The order of proteins in a chimeric fusion can affect both the function of the expressed proteins and their ability to interact, in this case with each other. Efficient expression, folding, secretion, protease cleavage and overall performance of the fusion protein may also be affected by the order in which the BMP protein and inhibitor protein are assembled on the transgene expression system. The nature of the amino acids incorporated in the linker region between proteins expressed in a fusion construct (eg. hydrophobic, acidic, basic, etc.) can also have an effect on heterologously expressed fusion protein performance. The length of the linker region must also be of sufficient length to allow Noggin, or a similarly incorporated inhibitor, sufficient degrees of freedom to allow the inhibitor access to the BMP binding site and access to the protease cleavage site.

The biological BMP/Noggin complex in the PDB entry 1M4U crystal structure is defined to be a 2:2 BMP:Noggin complex. The interatomic distances and examples recited below are based on the 1M4U structure (Groppe et al. Nature 2002; 420:636-642) as the biologically relevant BMP:Noggin complex and are not intended to limit the embodiment, but rather to provide an examplary system. Additional embodiments for BMP/BMP-inhibitor fusion proteins are possible and not limited to these examples.

BMP residues numbered 28-139 corresponds to sequence:

28-Glu-Asp-Ser-Ser-Asp . . . Ala-Cys-Gly-Cys-His-
139

Cys136 and Cys138 are part of the disulfide bond network that forms a cystine knot responsible for stabiliziing BMP.

The Noggin protein residues numbered 27-232 corresponds to sequence:

28-Gln-His-Tyr-Leu-His . . . Glu-Cys-Lys-Cys-Ser-
Cys-232

Cys228 and Cys230 are both involved in disulfide bonds. Cys232 is not disulfide bonded, but is close enough to the second molecule of noggin in the crystallographic complex to suggest that it could form a disulfide bond in the Noggin dimer.

Several options for the construction of a BMP/BMP-inhibitor fusion protein are discussed below. The following options, are based on the considerations addressed above, and are presented in order to illustrate the invention by way of example, and not by way of limitation.
Option 1: Molecule A of BMP dimer fused with Molecule A of Noggin dimer. One possible binding conformation between BMP and Noggin is represented by the interaction between BMP molecule A and Noggin molecule A in the 1M4U crystal structure (Groppe et al. Nature 2002; 420:636-642). The closest distance from BMP molecule A His139 to Noggin molecule A Gln28 is 18 Angstroms, However, this distance does not take into account the fact that the amino acid linker would have to loop around a helix in BMP in order to reach Noggin. Therefore, a distance on the order of 20-25 Angstroms, or greator, is more likely to provide the flexibility required for efficient binding between the two proteins.
Option 2: Molecule A of BMP dimer fused with molecule B of Noggin dimer. A second possible binding conformation between BMP and Noggin is represented by the interaction between BMP molecule A and Noggin molecule B in the 1M4U crystal structure (Groppe et al. Nature 2002; 420:636-642). The shortest distance from BMP molecule A His139 to Noggin molecule B Gln28 is 27.5 Angstroms. As stated above, this distance is a minimal estimate of the length required, and the optimal distance will likely be longer.
Potential problems with options 1 and 2. Because Cys138 of BMP is disulfide bonded, there may be limited flexibility near the N-terminus of the linker region. Since His139 of BMP is almost completely buried in the BMP dimer interface, homodimerization of BMP might be inhibited by adding a linker to His139. Based on this observation, it is likely that option 2 would present fewer problems with BMP homodimerization than option 1. On the other hand, reducing the dimerization of BMP may provide an additional biological advantage. First, monomeric BMP lacks biological activity, which is desirous at this point in the process. Second, if dimerization of BMP is hindered, the strength of the interaction with noggin might be reduced, which may assist in dissociation of BMP from the Noggin inhibitor during the BMP activation/purification process.

The primary difference between Options 1 and 2 is the length of the linker. Based on an analysis of the crystal structure, a beneficial embodiment would be to create a linker that spans at least ˜28 Angstroms, with a protease cleavage site near the C-terminus of the linker region. For both options 1 and 2, the protease cleavage site within the linker region should be as close to Noggin Gln28 as possible in order to make it accessible to a protease for enzymatic cleavage.
Option 3: Noggin-BMP fusion protein. A third variation of the fusion protein involves expression of Noggin, followed by BMP, and separated by a linker segment. Linking Noggin Cys232 to BMP Glu28 requires a linker that spans a minimum of 50 Angstroms. A cleavage site in the linker region near BMP Glu28 should facilitate accessibility of a proteolytic enzyme, and represent a better position than Noggin Cys232, which based on structural analysis could be problematic.

The feasibility of these fusion proteins is supported in the literature by the reports of active TGFβ (Andrades et al. Exp. Cell Res. 1999; 250:485-498) and BMP (Han et al. J. Orthop. Res. 2002; 20:747-755 and Schmoekel et al. Biotechnol. Bioeng. 2005; 89:253-262) fusion proteins.

Production of Transgenic Mammals Expressing BMP, BMP-Inhibitor, Furin-Resistant Mutant BMP and BMP/BMP-Inhibitor Fusion Proteins

As described above, transgenic protocols provide for the production of both chimeric and wholly transgenic animals. Both wholly transgenic offspring and chimeras may be used for breeding purposes. Where the creation of either a wholly transgenic or chimeric animal has been achieved, such as in the case of a transgenic animal capable of mammary expression of a recombinant BMP, such an animal can be crossbred with another transgenic animal, such as one producing a different recombinant BMP or a recombinant BMP-inhibitor, in order to create a transgenic capable of expressing both proteins. Furthermore, the production of transgenic animals capable of co-expressing any combination of recombinant BMP protein, protein variants, mutants, fusions or BMP-inhibitors is possible by crossbreeding either wholly transgenic or chimeric animals capable of expressing each. In addition, it is envisioned that the crossbreeding of an animal capable of expressing multiple recombinant BMPs or BMP-inhibitors could be crossbred with a second animal also capable of expressing multiple recombinant BMPs or BMP-inhibitors.

### EXAMPLES

The present invention is next described by means of the following examples. However, the use of these and other examples anywhere in the specification is illustrative only, and in no way limits the scope and meaning of the invention or of any exemplified form. Likewise, the invention is not limited to any particular preferred embodiments described herein. Indeed, many modifications and variations of the invention may be apparent to those skilled in the art upon reading this specification, and can be made without departing from its spirit and scope. The invention is therefore to be limited only by the terms of the appended claims, along with the full scope of equivalents to which the claims are entitled.

In accordance with the present invention there may be employed conventional molecular biology, microbiology, protein expression and purification, antibody, and recombinant DNA techniques within the skill of the art. Such techniques are explained fully in the literature. See, e.g., Glover and Hames, eds. DNA Cloning: A Practical Approach Vol I. Oxford University Press, 1995; Glover and Hames, eds. DNA Cloning: A Practical Approach Vol II. Oxford University Press, 1995; Glover and Hames, eds. DNA Cloning: A Practical Approach Vol III. Oxford University Press, 1996; Glover and Hames, eds. DNA Cloning: A Practical Approach, Vol IV. Oxford University Press, 1996; Gait, ed. Oligonucleotide Synthesis. 1984; Hames and Higgens, eds. Nucleic Acid Hybridization. 1985; Hames and Higgens, eds. Transcription And Translation. 1984; Perbal, A Practical Guide To Molecular Cloning. 1984; Ausubel et al., eds. Current Protocols in Molecular Biology. John Wiley & Sons, Inc. 1994; Sambrook et al. Molecular Cloning: A Laboratory Manual, Third Edition. Cold Spring Harbor Laboratory Press. 2001; Harlow and Lane. Using Antibodies: A Laboratory Manual. Cold Spring Harbor Laboratory Press. 1999; Dieffenbach and Dveksler, eds. PCR Primer: A Laboratory Manual, Second Edition. Cold Spring Harbor Laboratory Press. 2003; Hockfield et al. Selected Methods for Antibody and Nucleic Acid Probes. Cold Spring Harbor Laboratory Press. 1993; Gosling, ed. Immunoassays: A Practical Approach. Oxford University Press. 2000; Wilkinson, ed. In Situ Hybridization: A Practical Approach. Oxford University Press. 1999; Ashley, ed. PCR 2: A Practical Approach. Oxford University Press. 1996; Herrington and O'Leary, eds. PCR 3: PCR In Situ Hybridization: A Practical Approach. Oxford University Press. 1998; and Allan, ed. Protein Localization by Fluorescence Microscopy: A Practical Approach. Oxford University Press. 2000.

### Example 1

### Production of Recombinant Human BMP-2 in Transgenic Goats

### Materials and Methods

Assembly of the expression construct pBC1-GβCasSS-hBMP2: In this expression construct, the human BMP-2 pro-peptide coding sequence is under the transcriptional control of a strong β-casein promoter to direct expression of recombinant human BMP-2 in the mammary gland, and linked to a β-casein signal sequence to direct secretion of recombinant BMP-2 into milk produced by the mammary gland.

The human BMP-2 cDNA is PCR amplified from a commercially available cDNA clone (ATCC #U2OS-39) with a sense primer GβCasSS-hBMP2.F1 (5′ ATA TTC TCG AGA GCC ATG AAG GTC CTC ATC CTT GCC TGT CTG GTG GCT CTG GCC CTT GCA AGA GGC GCG GCT GGC CTC GTT CC 3′) (SEQ ID NO: X) containing an XhoI restriction endonuclease site (underlined), goat β-casein signal sequence (italic), and a partial 5′ human BMP-2 sequence (in bold); and an antisense primer, hBMP2.R1 (5′ CTA TGA CTC GAG TTT GCT GTA CTA GCG ACA CCC 3′) (SEQ ID NO: X) containing an XhoI site (underlined) and partial 3′ human BMP-2 sequence (in bold). The complete cDNA sequence of cDNA clone ATCC #U2OS-39 is set forth in SEQ ID NO: 1.

The 1.2 kb hBMP-2 PCR product is XhoI digested and subcloned into pGEM-T easy vector (Promega), to give the construct named pGEM-GβCasSS-hBMP2. The GβCasSS-hBMP2 insert of pGEM-GβCasSS-hBMP2 is excised by digestion with XhoI, purified with GFX matrix (Pharmacia Biotech, Baie d'Urfé, PQ, Canada) and ligated with XhoI-digested pBC1 (Invitrogen) to generate pBC1-GβCasSS-hBMP2.

pBC1-GβCasSS-hBMP2 is digested with NotI and SalI, and the resultant NotI/SalI-digested linear DNA, free of bacterial sequences, is prepared and used to generate transgenic goats. Briefly, circular expression construct DNA is purified by the cesium chloride gradient technique. This purified DNA is restricted with NotI and SalI, electrophoresed, and the linear DNA fragment is gel purified. The DNA fragment is then mixed with cesium chloride and centrifuged at 20° C., 60,000 rpm for 16 to 20 hrs in a Beckman L7 ultracentrifuge using a Ti70.1 rotor (Beckman Instruments, Fullerton, Calif., USA). The DNA band is removed, dialyzed against WFI water for 2-4 hrs, and precipitated in ethanol. The precipitated DNA is resuspended in injection buffer (5 mM Tris pH 7.5, 0.1 mM EDTA, 10 mM NaCl) and dialyzed against the same buffer at 4° C. for 8 hrs. Two additional dialysis steps are performed, one for 16 hrs and the second for at least 8 hrs. After dialysis the DNA was quantitated using a fluorometer. Prior to use an aliquot is diluted to 2-3 ng/ml in injection buffer.

Hormonal treatment of oocyte donor goats: Recipient and donor crossbreed goats (mainly Saanen×Nubian) are estrus synchronized by means of an intravaginal sponge impregnated with 60 mg medroxyprogesterone acetate (Veramix®, Pharmacia Animal Health, Ontario, Canada) for 10 days, together with a luteolytic injection of 125 μg clorprostenol (Estrumate®, Schering, Canada) administered intramuscularly 36 hours prior to sponge removal. In addition, for donor goats follicular development is stimulated by a gonadotrophin treatment consisting of 70 mg NIH-FSH-P1 (Folltropin-V®, Vetrepharm, Canada) and 300 IU eCG (Novormon 5000®, Vetrepharm, Canada) administered intramuscularly 36 h prior to Laparaoscopic Ovum Pick-Up (LOPU).

Collection of Cumulus Oocyte Complexes (COCs) from donor goats by Laparoscopic Ovum Pick-Up (LOPU): Cumulus oocyte complexes (COCs) from donor goats are recovered by aspiration of follicle contents (puncture or folliculocentesis) under laparoscopic observation. The laparoscopy equipment used (Richard Wolf, Germany) is composed of a 5 mm telescope, a light cable, a light source, a 5.5 mm trocar for the laparoscope, an atraumatic grasping forceps, and two 3.5 mm “second puncture” trocars. The follicle puncture set is composed of a puncture pipette, tubing, a collection tube, and a vacuum pump. The aspiration pipette is made using an acrylic pipette (3.2 mm external diameter, 1.6 mm internal diameter), and a 20G short bevel hypodermic needle, which is cut to a length of 5 mm and fixed into the tip of the pipette with instant glue. The connection tubing is made of clear plastic tubing with an internal diameter of 5 mm, and connected the puncture pipette to the collection tube. The collection tube is a 50 ml centrifuge tube with an inlet and an outlet available in the cap. The inlet is connected to the aspiration pipette, and the outlet is connected to a vacuum line. Vacuum is provided by a vacuum pump connected to the collection tube by means of clear plastic 8 mm tubing. The vacuum pressure is regulated with a flow valve and measured as drops of collection medium per minute entering the collection tube. The vacuum pressure is typically adjusted to 50 to 70 drops per minute.

The complete puncture set is washed and rinsed 10 times with tissue culture quality distilled water before gas sterilization, and one time before use with collection medium [M199+25 mM HEPES (Gibco) supplemented with penicillin, streptomycin, kanamycin, bovine serum albumin, and heparin]. Approximately 0.5 ml of this medium is added to the collection tube to receive the oocytes.

Donors are deprived of food for 24 hours and of water for 12 hours prior to surgery. The animals are pre-anesthetized by injection of diazepam (0.35 mg/kg body weight) and ketamine (5 mg/kg body weight). Thereafter, anesthesia is maintained by administration of isofluorane via endotrachial intubation. Preventive antibiotics (e.g., oxytetracycline) and analgesic/anti-inflammatories (e.g., flunixine) are administered by intramuscular injection in the hind limbs. The surgical site is prepared by shaving the abdominal area, then scrubbing first with soap and water and then with a Hibitaine:water solution, followed by application of iodine solution.

A small incision/puncture is made with a scalpel blade about 2 cm cranial from the udder and about 2 cm left from the midline. The 5 mm trocar is inserted and the abdominal cavity is inflated with filtered air through the trocar sleeve gas valve. The laparoscope is inserted into the trocar sleeve. A second incision is made about 2 cm cranial from the udder and about 2 cm right from the midline, into which is inserted a 3.5 mm trocar. The trocar is removed, and the forceps are inserted. A third incision is made about 6 cm cranial to the udder and about 2 cm right from the midline. The second 3.5 mm trocar and trocar sleeve is inserted into this incision. The trocar is removed and the aspiration pipette connected to the vacuum pump and the collection tube is inserted therein.

After locating the reproductive tract below the bladder, the ovary is exposed by pulling the fimbria in different directions, and the number of follicles available for aspiration is determined. Generally, follicles greater than 2 cm are considered eligible for aspiration. The follicles are punctured one by one and the contents aspirated into the collection tube under vacuum. The needle is inserted into the follicle and rotated gently to ensure that as much of the follicle contents as possible are aspirated. After >10 follicles are aspirated and/or before moving to the other ovary, the pipette and tubing are rinsed using collection media from a sterile tube.

In vitro maturation of oocytes collected by LOPU: To each collection tube containing cumulus oocyte complexes (COCs) is added about 10 ml of searching medium [EmCare® (PETS, cat. # ECFS-100) supplemented with 1% heat inactivated Fetal Bovine Serum (FBS)]. The resulting solution is aspirated into a grid search plate and transferred to Petri dishes containing the same medium for the purpose of scoring each COC for amount and expansion of cumulus. The COCs are then washed with in vitro maturation (IVM) medium; (M199+25 mM HEPES supplemented with bLH, bFSH, estradiol β-17, pyruvate, kanamycin and heat-inactivated EGS) that has been equilibrated in an incubator under 5% CO₂ at 35.5° C. for at least 2 hours. The COCs are pooled in groups of 15-25 per droplet of IVM medium, overlayed with mineral oil, and incubated in 5% CO₂ at 35.5° C. for 26 hours.

Preparation of semen for in vitro fertilization: Fresh semen is collected from 2 adult Saanen males of known fertility. After collection, sperm capacitation is achieved as follows. A 5 μl aliquot of fresh semen is diluted in 500 μl warm modified Defined Medium (mDM) comprising NaCl, KCl, NaH₂PO₄.H₂O, MgCl₂.6H₂O, CaCl₂.2H₂O, glucose, 0.5% phenol red, Na-Pyruvate, NaHCO₃, gentamicin, and BSA. The solution is allowed to stand at room temperature in the absence of light for 3 hours. An additional 1 ml of mDM solution is added and 100 μl of the resulting solution is overlaid on a 45%:90% Percoll® gradient [Percoll® (Sigma P1644) in modified Sperm Tyrodes Lactate (SPTL) solution] in a conical centrifuge tube. The solution is centrifuged on the Percoll gradient at 857×g for 30 minutes. The pellet is resuspended in mDM solution and centrifuged at the same speed for 10 minutes. The pellet is re-suspended in capacitation medium (mDM, supplemented with 8b-cAMP, lonomycin, and Heparin). The resuspended semen is cultured at 38.5° C. under 5% CO₂ for 15 minutes. The sperm concentration is then adjusted to final concentration of 20×10⁶ sperm/ml by addition of mDM solution.

In vitro fertilization of oocytes: The expanded cumulus cells are partially removed from the matured COCs by pipetting repeatedly through two fine-bore glass pipettes (200 and 250 μm internal diameter), leaving one layer of cumulus cells on the zona. The oocytes are washed with in vitro fertilization (IVF) medium, a modified Tyrode's albumin lactate pyruvate (TALP), and transferred to 40 μl droplets of the same medium (15-20 oocytes per 40 μl droplet) under mineral oil. A 5 μl aliquot of the capacitated sperm suspension (20×10⁶ sperm/ml), prepared as described in Example 4.4., is added to each 40 μl droplet. The inseminated oocytes are cultured at 38.5 C in 5% CO₂ for 15-16 hours.

Pronuclear microinjection of oocytes: After culturing for 15-16 hours, the cumulus cells are stripped from the inseminated oocytes (zygotes) by repeated pipetting as described above. The zygotes are then observed for pronuclear formation using an Olympus stereomicroscope. To improve pronucleus visualization, the zygotes are washed in EmCare® (PETS, cat. # ECFS-100) supplemented with 1% Fetal Bovine Serum (FBS), (Gibco BRL, Australian or New Zealand sourced, heat inactivated at 56 ° C. for 30 minutes), then centrifuged at 10,400×g for 3 minutes before observation. Zygotes with visible pronuclei are selected for microinjection and transferred to 50 μl droplets of temporary culture medium (INRA Menezo B2, Meditech cat. #CH-B 04001 supplemented with 2.5% FBS) during manipulation. The zygotes are then transferred to 50 μl droplets of EmCare®+1% FBS (about 20 zygotes per droplet) and microinjected with linear GβCas-hBMP2 fragment (3 ng/ml of the DNA in a buffer of 5 mM Tris, 0.1 mM EDTA. 10 mM NaCl buffer, pH 7.5). The injected zygotes are washed and cultured in temporary culture medium to await transfer to recipients.

Transfer of embryos to oviduct of recipient goats and birth of kids: Adult goats of various breeds including the Boer, Saanen, and Nubian breeds are used as recipients. They are estrus synchronized by means of an intravaginal sponge impregnated with 60 mg medroxyprogesterone acetate (Veramix®, Pharmacia Animal Health, Ontario, Canada) left in place for 9 days, together with a luteolytic injection of 125 μg clorprostenol (Estrumate®, Schering, Canada) and 500 IU eCG (Novormon 5000®, Vetrepharm, Canada) administered intramuscularly 36 hours prior to sponge removal . Sponges are inserted into the recipient goats on the same day as the donor goats but removed approximately 15 hours earlier. Each recipient is subsequently treated with an intramuscular injection of 100 μg GnRH (Factrel®, 2.0 ml of 50 μg/ml solution), 36 hours after sponge removal. The recipients are tested for estrus with a vasectomized buck at 12 hour intervals beginning 24 hours after sponge removal and ending 60-72 hours after sponge removal.

Recipient goats are fasted, anesthetized, and prepared for surgery following the same procedures previously described for donor goats. They also receive preventive antibiotic therapy and analgesic/anti-inflammatory therapy, as described for donors. Prior to surgery, a laparoscopic exploration of each eligible recipient is performed to confirm that the recipient has one or more recent ovulations (as determined by the presence of corpora lutea on the ovary), and a normal oviduct and uterus. The laparoscopic exploration is carried out to avoid performing a laparotomy on an animal which had not responded properly to the hormonal synchronization protocol described above. Two incisions are made (one 2 cm cranial to the udder and 2 cm left of the midline, and the other 2 cm cranial to the udder and 2 cm right of the midline) and the laparoscope and forceps are inserted as described above. The ovaries are exposed by pulling up the fimbria with the forceps, and the number of ovulations present as well as the number of follicles larger than about 5 mm diameter are noted. Recipients with at least one ovulation present and having a normal uterus and oviduct are eligible for transfer. A mid-ventral laparotomy incision of approximately 10 cm length is established in eligible recipients, the reproductive tract is exteriorized, and the embryos are implanted into the oviduct ipsilateral to the ovulation(s) by means of a TomCat® catheter threaded into the oviduct from the fimbria. The incisions are closed and the animal is allowed to recover in a post-op room for 3 days before being returned to the pens. Skin sutures are removed 7-10 days after surgery.

Recipients are scanned by transrectal ultrasonography using a 7.5 Mhz linear array probe to diagnose pregnancy at 28 and 60 days after transfer.

Newborn kids are removed from does at birth to prevent disease transmission from doe to kid by ingestion of doe's raw colostrum and/or milk, exposure to doe's fecal matter or other potential sources of disease. Kids are fed thermorized colostrum for the first 48 hours of life, and pasteurized doe milk thereafter until weaning.

Identification of transgenic goats: Blood and tissue samples are taken from putative transgenic kids at approximately 4 days after birth, and again at approximately two weeks after birth. At each sampling interval, about 2-7 ml blood sample is collected from each kid into an EDTA vacutainer, and stored at 4° C. for up to 24 hours until use. Tissue samples are obtained by clipping the ear tip of each kid, and stored at 20° C. until use. Genomic DNA is isolated from the blood samples using a QIAamp® DNA Blood Mini Kit (Qiagen, Cat. # 51106), and from the tissue samples using DNeasy® Tissue Kit (Qiagen, cat #69506). For each sample, the DNA is eluted in 150-200 μl 0.1× buffer AE and stored at 4° C. until ready to use.

PCR screening is performed on each DNA sample to determine the presence of the BMP-2-encoding transgene. Genomic DNA samples are diluted using nuclease-free water to a concentration of 5 ng/μl. A 20 μl portion of the diluted DNA is added to a 0.2 ml Ready-To-Go™ PCR tube containing a PCR bead, together with 5 μl 5× primer mix containing dUPT (Amersham Bioscience, cat. #272040) and UDG (Invitrogen, cat. #18054-015).

Primers GβCasSS-hBMP2.F2 (5′ CTG GCC CTT GCA AGA GGC GCG GCT GG 3′) (SEQ ID NO: X), which spans the junction of the goat β-casein signal sequence (italics) and the 5′ end of the human BMP-2 sequence (in bold) within the transgene, and hBMP2.R2 (5′ TGC TGG GGG TGG GTC TCT G-3′ (SEQ ID NO: X), a reverse primer within the hBMP2 coding sequence, amplify a 169 bp fragment from the genomic DNA of transgenic animals.

Another primer set, βCas.F1 (5′ GAG GAA CAA CAG CAA ACA GAG 3′) (SEQ ID NO: X) and βCas.R1 (5′ ACC CTA CTG TCT TTC ATC AGC 3′) (SEQ ID NO: X), which amplifies a 360 bp portion of the endogenous goat β-casein gene, serves as in internal positive control to indicate that the extracted DNA can be amplified by PCR.

The sample is subjected to thermal cycling and then applied to a 1% agarose gel. Negative controls (genomic DNA isolated from non-transgenic animals) and positive controls (genomic DNA from non-transgenic animals spiked with the microinjected linear GβCasSS-hBMP2 fragment) are also included. Samples which exhibit a band corresponding to the positive control are deemed positive.

Confirmation of transgene presence, and estimation of transgene copy number, is also performed using Southern blotting analysis with Boehringer Mannheim's DIG system. Genomic DNA (5 μg) extracted from blood and tissue is digested with ApaLI. This digestion is followed by gel electrophoresis and Southern transfer to nylon membranes (Roche Diagnostics Canada). The blot is hybridized in a DIG Easy Hyb™ buffer (Roche Diagnostics Canada) at 42° C. overnight using an insulator probe labeled by the PCR DIG probe synthesis kit (Roche Diagnostics Canada), which hybridizes at the 5′ end of the transgene. This insulator probe is PCR amplified from the pBC1-GβCasSS-hBMP2 construct using the primers InsF1 (5′ TGC TCT TTG AGC CTG CAG ACA CCT 3′) (SEQ ID NO: X) and InsR1 (5′ GGC TGT TCT GAA CGC TGT GAC TTG 3′) (SEQ ID NO: X). The membrane is washed, detected by the CDP-Star™ substrate (Roche Diagnostics Canada) and visualized by the FluorChem™ 8000 System (Alpha Innotech Corporation). The size of the genomic DNA fragment detected by this probe will vary depending on the site of integration.

The same membrane is stripped with stripping buffer (Roche Diagnostics Canada) and re-hybridized with a DIG-labeled PCR probe hybridizing within the BMP sequence. The 1.15 kb probe was PCR amplified from the pBC1-GβCasSS-hBMP2 construct using the primers hBMP2.F1 (5′ GGC GCG GCT GGC CTC GTT CC 3′) (SEQ ID NO: X) and hBMP2.R3 (5′ TTT GCT GTA CTA GCG ACA CCC 3′) (SEQ ID NO: X).

Upon analysis, the expected size bands are detected for transgenic offspring and copy number is estimated.

Fluorescent in situ hybridization (FISH) is performed as described in Keefer et al. Biol. Reprod. 2001; 64:849-856 in order to determine the number of chromosomal integration sites.

Induction of lactation and collection of milk: Transgenic female founders (F0 generation) are induced to lactate at 3-4 months of age in order to confirm the expression of recombinant hBMP-2 in milk. For such purpose, the goats are hormonally stimulated with estradiol cypionate (0.25 mg/KBW) and Progesterone (0.75 mg/KBW) every 48 hrs for two weeks, followed by treatment with dexamethasone (8 mg/goat/day) for 3 days. In general, milk production starts during the dexamethasone treatment and the animals are milked twice per day for as long as necessary to produce enough material for further testing.

Milk is collected by hand milking or using conventional, commercially available milking equipment. The milk is centrifuged at 3000×g for 30 minutes at 4° C., and the resultant whey phase is separated from the fat phase and precipitates. The whey phase is stored at −70° C. until analysis.

Purification of BMP-2: The recombinant hBMP-2 protein is purified from skimmed milk of transgenic goats by heparin affinity chromatography as previously described (see, for example, Vallejo et al. J Biotech 2002; 94:185-194).

Purification is completed at room temperature (ca. 20 C). Skimmed milk is dialyzed against 4 M urea, 20 mM Tris-HCl (pH 8.0) and passed through a 0.45 um filter (Minisart, Sartorius, Gottingen, Germany). 12 mg of total protein is applied to a 10 ml HiTrap heparin-sepharose column (Pharmacia, Upsala, Sweden), equilibrated beforehand with 5 column volumes (CV) of dialysis buffer. The column is washed again with 5 CV of dialysis buffer and the recombinant hBMP-2 eluted with a two step NaCl gradient: 350 and 500 mM respectively. Eluted fractions are concentrated to 1 g/L by ultrafiltration (10 kDa vivaspin, Binbrook, UK) and stored at −70 C. Removal of salt and urea from the eluted fractions containing pure recombinant hBMP-2 is carried out by buffer exchange with 50 mM MES (2-[N-morpholino]ethanesulfonic acid; pH 5.0) through semi-continuous diafiltration (5 kDa vivaspin, Vivascience). For long-term storage, recombinant hBMP-2 is freeze dried in 50 mM MES (pH 5.0) under standard conditions. The freeze-dried rhBMP-2 is rehydrated without loss of biological activity.

Electrophoresis under non-reducing conditions is performed using with precast 8-16% SDS-PAGE gels (Criterion, BioRad, Hercules, USA) according to manufacturer's instructions. The gels are stained with Coomassie Brillant Blue and subjected to densitometry analysis (Quantiscan, Biosoft, Ferguson, USA). A commercially available purified recombinant hBMP-2 (R&D Systems, Minneapolis, USA) is used as a standard for densiometric quantification. The protein concentration of the standard is determined by UV280 with a calculated molar extinction coefficient of 18,860 M⁻¹cm⁻¹.

N-terminal sequence analysis from blotted protein bands is carried out by automated Edman degradation (Protein Sequencer 470 A, Applied Biosystems, Foster City, Calif.) and on line HPLC (12A, Applied Biosystems) to confirm that the recombinant protein represents BMP-2.

BMP-2 ELISA assay: The level of recombinant hBMP-2 protein in skimmed milk of transgenic goats, and/or of purified recombinant hBMP-2 protein isolated from skimmed milk by heparin affinity chromatography, is quantitated using the BMP-2 Quantikine ELISA kit (R&D Systems, catalog # DBP200) as per the manufacturers instructions.

In vitro BMP-2 activity assay: alkaline phosphatase induction in C2C12 cells: The activity of recombinant hBMP-2 protein is quantitated based upon induction of alkaline phosphatase in in vitro cultured C2C12 cells, as has been described (see, for example, Peel et al. J Craniofacial Surg. 2003; 14:284-291 and Hu et al. Growth Factors. 2004; 22:29033).

C2C12 cells (ATCC accession number CRL-1772, Manassas, Va.) are passaged before confluent and resuspended at 0.5×10⁵ cells/ml in MEM supplemented with 15% heat-inactivated fetal bovine serum, antibiotics and 50 μg/ml ascorbic acid. One ml of cell suspension is seeded per well of a 24 well tissue culture plate (BD Falcon, Fisher Scientific Cat # 08-772-1). An aliquot of test BMP-2 sample is added and the cultures maintained at 37° C. and 5% CO₂. Test BMP-2 samples included whey phase of skimmed milk from transgenic goats, purified recombinant hBMP-2 isolated from whey phase of skimmer milk from transgenic goats by heparin affinity chromatography, and as a positive control a commercially available purified recombinant hBMP-2 (R&D Systems, Minneapolis, USA). Control cultures (cultured in media without added BMP-2 sample) are cultured for 2 to 7 days. Medium is changed every two days.

At harvest cultures are rinsed with Tris buffered saline (20 mM Tris, 137 mM NaCl, pH 7.4) and M-Per lysis buffer (Pierce Biotechnology Inc., Rockford, Ill., catalogue # 78501) is added. The cell layer is scraped into Eppendorf tubes and sonicated. The lysate is centrifuged at 5000 g at 5° C. for 10 minutes, and the supernatant assayed for alkaline phosphatase (ALP) by monitoring the hydrolysis of nitrophenol phosphate in alkaline buffer (Sigma-Aldrich, St. Louis Mo., catalog P5899) as described in Peel et al. J Craniofacial Surg. 2003; 14:284-291 or by using the Alkaline Phosphatase detection kit, Fluorescence (Sigma-Aldrich, catalogue #APF) according to manufacturer's instructions. To normalize the ALP activity the cellular protein content in each well is also assayed using the Coomasie (Bradford) Protein Assay (Pierce Biotechnology Inc., catalogue # 23200). The normalized ALP activity for each sample is calculated by dividing the ALP activity per well by the protein content per well. An activity score is calculated by dividing the ALP activity for each sample by the mean ALP activity of the control and is compared to the score achieved by the positive BMP control.

In vivo BMP-2 activity assay: osteoinduction in mice: The osteoinductive capacity of recombinant hBMP-2 protein is measured using the mouse implantation model of osteoinduction, which has been described (see, for example, Urist et al. Meth Enzym. 1987:146; 294-312).

Test BMP-2 samples include whey phase of skimmed milk from transgenic goats, purified recombinant hBMP-2 isolated from whey phase of skimmed milk from transgenic goats by heparin affinity chromatography, and as a positive control a commercially available purified recombinant hBMP-2 (R&D Systems, Minneapolis, USA). BMP-2 samples are co-lyophilized with atelopeptide type I collagen carrier (Collagen Corp Paulo Alto Calif.) to produce BMP-2 implants.

Swiss-Webster mice (Harlan Sprague-Dawley, Indianapolis, Ind.) are anesthetized (ether, Mallinckrodt, Paris, Ky.) and placed on the table in a prone position. A 1 by 2 cm site is shaved in the dorsum of the lumbar spine extending over both hips. The site is prepared with 70% alcohol solution. A 10 mm skin incision is made perpendicular to the lumbar spine and muscle pouches were created in each hind quarter. The BMP-2 implant, placed in no. 5 gelatin capsules (Torpac Inc. Fairfield, N.J.), is implanted in the muscle pouches and the wounds closed with metal clips (Poper, Long Island, N.Y.).

Animals receive a BMP-2 capsule implant in one hind quarter muscle mass, with the contralateral muscle mass being implanted with the collagen carrier alone.

The animals are killed at 4 weeks post-implantation and the hind quarters are disarticulated for radiographic examinations (Faxitron, Field Emmission Corporation, McMinnville, Oreg.; 25 kVp, 0.6 sec.). The specimens are fixed in buffered neutral 10% formalin for 24 hours. The implants are excised and embedded in paraffin. Ten micron sections are prepared and stained with hematoxylin-eosin and azure II. Hematoxylin-eosin von Kossa's staining is used to identify sites of calcification.

Microradiographs of histologically valid bone deposits are analyzed by using Image Pro Plus image analysis software (Media Cybernetics, Inc., Silver Spring, Md.) as has been described (see, for example, Becker et al. J Periodontol 1996; 67:1025-1033 and Kawai and Urist. Clin. Orthop. Relat. Res. 1988; 233:262-267). The radiopaque area of the implant is expressed as a percentage of the total area of adjacent tissues of the ipsilateral femur. Histomorphometric methods are applied by using the same image analysis software. The volume of new bone and cartilage formed is compared with the total volume of the implant and expressed as a percentage.

The inventors have improved the quantitation of induced heterotropic bone formation in mice by using a micro-CT scanner. The hind quarters are imaged using a microCT scanner (eXplore Locus, GE Healthcare, London, ON, CANADA). The implant is localized and the volume of new bone and the mineral content of each implant is determined using the bone analysis software provided by the manufacturer. This method is more sensitive and provides better resolution than microradiographs and provides volume measurements compared to area measurements provided by microradiographs or histological analysis. Consequently the quantitation of induced bone using microCT is more accurate than that estimated from microradiographs.

### Results and Discussion

This example describes methods to generate the expression construct pBC1-GβCasSS-hBMP2. This expression construct is used to generate a linear GβCasSS-hBMP2 fragment, which was used to generate transgenic goats via the microinjection technique. The linear GβCasSS-hBMP2 fragment used to generate transgenic goats contains, in this order: 1) dimerized chicken β-globin gene insulator; 2) goat β-casein promoter; 3) goat β-casein exon 1, intron 1, and partial exon 2; 4) an XhoI cloning site; 5) β-casein signal sequence; 6) a hBMP-2 coding sequence; 6) a STOP codon; 7) β-casein partial exon 7, intron 7, exon 8, intron 8 and exon 9; and 8) additional β-casein 3′ genomic sequence.

The presence of the hBMP-2 transgene in founder (F0 generation) goats is confirmed by PCR and Southern blotting, and the presence of recombinant hBMP-2 in the milk of lactating goats is confirmed by ELISA. A permanent line of transgenic goats is established by breeding of the founder (F0) generation goats (either to non-transgenics goats, or by cross-breeding of an F0 male and F0 female).

Milk is collected from the transgenic goats. Recombinant hBMP-2 protein is purified from the collected milk by heparin affinity chromatography.

The biological activity of the recombinant hBMP-2 (either in crude form as the whey phase of skimmed milk from transgenics or in pure form following purification by heparin affinity chromatography) is verified and quantitated by both in vitro (alkaline phosphatase induction in C2C12 cells) and in vivo (osteoinduction in mice) techniques.

### Example 2

### Production of Recombinant Human BMP-7 in Transgenic Goats

### Materials and Methods

Assembly of the expression construct pBCI-GβCasSS-hBMP7: In this expression construct, the human BMP-7 coding sequence is under the transcriptional control of a strong β-casein promoter to direct expression of recombinant human BMP-7 in the mammary gland, and linked to a β-casein signal sequence to direct secretion of recombinant BMP-7 into milk produced by the mammary gland.

The human BMP-7 cDNA is PCR amplified from a cDNA clone (ATCC Number 68182 or ATCC Number 68020). PCR is performed using the primers hBMP7mut.F1 (5′ ATA TTT CTC GAG GAC TTC AGC CTG GAC AAC GAG GTG CAt TCG AGC TTC ATC CAC 3′) (SEQ ID NO: X) containing an XhoI restriction endonuclease site (underlined) and a partial human BMP-7 sequence (bold) with a nucleotide change at one position (lowercase) (in order to destroy the ApaLI and XhoI sites in the BMP-7 coding sequence, while maintaining a Histidine residue at that position in the encoded amino acid sequence) and hBMP7.R1 (5′ CTA TGA CTC GAG CTC GGA GGA GCT AGT GGC AG 3′) (SEQ ID NO: X) containing an XhoI site (underlined) and partial 3′ human BMP-7 sequence (in bold). The 1.24 kb hBMP-7mut PCR product is XhoI digested and subcloned into pGEM-T easy vector (Promega), to give the construct named pGEM-hBMP7mut.

The hBMP7 coding sequence for use in the transgene construct is then PCR amplified from the pGEM-hBMP7mut plasmid with a sense primer GβCasSS-hBMP7.F1 (5′ ATA TTC TCG AGA GCC ATG AAG GTC CTC ATC CTT GCC TGT CTG GTG GCT CTG GCC CTT GCA AGA GAC TTC AGC CTG GAC AAC 3′) (SEQ ID NO: X) containing an XhoI restriction endonuclease site (underlined), goat β-casein signal sequence (italic), and a partial human BMP-7 sequence (in bold); and an antisense primer, hBMP7.R1 (5′ CTA TGA CTC GAG CTC GGA GGA GCT AGT GGC AG 3′) (SEQ ID NO: X) (SEQ ID NO: X) containing an XhoI site (underlined) and partial 3′ human BMP-7 sequence (in bold).

The 1.3 kb hBMP-7 PCR product is XhoI digested and subcloned into pGEM-T easy vector (Promega), to give the construct named pGEM-GβCasSS-hBMP7. The GβCasSS-hBMP7 insert of pGEM-GβCasSS-hBMP7 is excised by digestion with XhoI, purified with GFX matrix (Pharmacia Biotech, Baie d'Urfé, PQ, Canada) and ligated with XhoI-digested pBC1 (Invitrogen) to generate pBC1-GβCasSS-hBMP7.

pBC1-GβCasSS-hBMP7 is digested with NotI and SalI, and the resultant NotI/SalI-digested linear DNA, free of bacterial sequences, is prepared and used to generate transgenic goats. Briefly, circular expression construct DNA is purified by the cesium chloride gradient technique. This purified DNA is restricted with NotI and SalI, electrophoresed, and the linear DNA fragment is gel purified. The DNA fragment is then mixed with cesium chloride and centrifuged at 20° C., 60,000 rpm for 16 to 20 hrs in a Beckman L7 ultracentrifuge using a Ti70.1 rotor (Beckman Instruments, Fullerton, Calif., USA). The DNA band is removed, dialyzed against WFI water for 2-4 hrs, and precipitated in ethanol. The precipitated DNA is resuspended in injection buffer (5 mM Tris pH 7.5, 0.1 mM EDTA, 10 mM NaCl) and dialyzed against the same buffer at 4° C. for 8 hrs. Two additional dialysis steps are performed, one for 16 hrs and the second for at least 8 hrs. After dialysis the DNA was quantitated using a fluorometer.

Generation of stably transfected cell lines: Primary fetal goat cells are derived from day 28 kinder fetuses recovered from a pregnant Saanen breed female goat, and cultured for 3 days prior to being cryopreserved. Chromosome number (2n=60) and sex analysis are performed prior to use of cells for transfection experiments. Under the culture conditions used, all primary lines should have a normal chromosome count indicating the absence of gross chromosomal instability during culture.

Stably transfected cell lines are generated using lipid mediated gene transfer. Female primary fetal goat cell lines were thawed and at passage 2, co-transfected with the linearized GβCasSS-hBMP7 fragment and the linearized pSV40/Neo selectable marker construct (Invitrogen). The pSV40/Neo linear fragment is generated by restriction of the vector with XbaI and NheI, followed by purification of the fragment as described above for GβCasSS-hBMP7. Stably transfected cell lines are selected with G418 and frozen by day 21 (day 0=transfection date). Multiple stably transfected clonal cell lines may be derived by this procedure.

For confirmation of transgene integration in stably transfected cells lines, genomic DNA is isolated from cell pellets using DNeasy® Tissue Kit (Qiagen, cat #69506). For each sample, the DNA is eluted in 150-200 μl 0.1× buffer AE and stored at 4° C. until ready to use.

PCR screening is performed on each DNA sample to determine the presence of the BMP-7-encoding transgene. Genomic DNA samples are diluted using nuclease-free water to a concentration of 5 ng/μl. A 20 μl portion of the diluted DNA is added to a 0.2 ml Ready-To-Go™ PCR tube containing a PCR bead, together with 5 μl 5× primer mix containing dUPT (Amersham Bioscience, cat. #272040) and UDG (Invitrogen, cat. #18054-015).

Primers GβCasSS-hBMP7.F2 (5′ CTG GCC CTT GCA AGA GAC TTC AGC CTG GAC AAC 3′) (SEQ ID NO: X), which spans the junction of the goat β-casein signal sequence (italics) and the 5′ end of the human BMP-7 sequence (in bold) within the transgene, and hBMP7.R2 (5′ CTC CAC CGC CAT CAT GGC GTT G-3′ (SEQ ID NO: X), a reverse primer within the hBMP-7 coding sequence, amplify a 207 bp fragment from the genomic DNA of transgenic animals.

Another primer set, βCas.F1 (5′ GAG GAA CAA CAG CAA ACA GAG 3′) (SEQ ID NO: X) and βCas.R1 (5′ ACC CTA CTG TCT TTC ATC AGC 3′) (SEQ ID NO: X), which amplifies a 360 bp portion of the endogenous goat β-casein gene, serves as in internal positive control to indicate that the extracted DNA can be amplified by PCR.

The sample is subjected to thermal cycling and then applied to a 1% agarose gel. Negative controls (genomic DNA isolated from non-transgenic animals) and positive controls (genomic DNA from non-transgenic animals spiked with the microinjected linear GβCasSS-hBMP7 fragment) are also included. Samples which exhibit a band corresponding to the positive control are deemed positive.

Confirmation of transgene presence, and estimation of transgene copy number, is also performed using Southern blotting analysis with Boehringer Mannheim's DIG system. Genomic DNA (5 μg) extracted from blood and tissue is digested with ApaLI. This digestion is followed by gel electrophoresis and Southern transfer to nylon membranes (Roche Diagnostics Canada). The blot is hybridized in a DIG Easy Hyb™ buffer (Roche Diagnostics Canada) at 42° C. overnight using an insulator probe labeled by the PCR DIG probe synthesis kit (Roche Diagnostics Canada), which hybridizes at the 5′ end of the transgene. This insulator probe is PCR amplified from the pBC1-GβCasSS-hBMP2 construct using the primers InsF1 (5′ TGC TCT TTG AGC CTG CAG ACA CCT 3′) (SEQ ID NO: X) and InsR1 (5′ GGC TGT TCT GAA CGC TGT GAC TTG 3′) (SEQ ID NO: X). The membrane is washed, detected by the CDP-Starm substrate (Roche Diagnostics Canada) and visualized by the FluorChem™ 8000 System (Alpha Innotech Corporation). The size of the genomic DNA fragment detected by this probe will vary depending on the site of integration.

The same membrane is stripped with stripping buffer (Roche Diagnostics Canada) and re-hybridized with a DIG-labeled PCR probe hybridizing within the BMP sequence. The 1.2 kb probe was PCR amplified from the pBC1-GβCasSS-hBMP7 construct using the primers hBMP7.F2 (5′ GAC TTC AGC CTG GAC AAC GAG GTG 3′) (SEQ ID NO: X) and hBMP7.R3 (5′ CTC GGA GGA GCT AGT GGC AG 3′) (SEQ ID NO: X).

Upon analysis, the expected size bands are detected for stably transfected cell lines and transgene copy number is estimated.

Fluorescent in situ hybridization (FISH) is performed as described in Keefer et al. Biol. Reprod. 2001; 64:849-856 in order to determine the number of chromosomal integration sites.

These stably transfected cell lines for which integration of the transgene is confirmed will serve as donor cells for nuclear transfer.

Oocyte donor and recipient goats: Intravaginal sponges containing 60 mg of medroxyprogesterone acetate (Veramix®) are inserted into the vagina of donor goats (Alpine, Saanen, and Boer cross bred goats) and left in place for 10 days. An injection of 125 μg cloprostenol is given 36 hrs before sponge removal. Priming of the ovaries is achieved by the use of gonadotrophin preparations, including FSH and eCG. One dose equivalent to 70 mg NIH-FSH-P1 of Ovagen™ is given together with 400 IU of eCG (Equinex) 36 h before LOPU (Laparoscopic Oocyte Pick-Up).

Recipients are synchronized using intravaginal sponges as described above for donor animals. Sponges are removed on day 10 and an injection of 400 IU of eCG is given. Estrus is observed 24-48 hrs after sponge removal and embryos are transferred 65-70 hrs after sponge removal.

Laparoscopic oocyte Pick-Up (LOPU) and embryo transfer: These procedures are performed essentially as described in Examples 1, above.

Donor goats are fasted 24 hours prior to laparoscopy. Anesthesia is induced with intravenous administration of diazepam (0.35 mg/kg body weight) and ketamine (5 mg/kg body weight), and is maintained with isofluorane via endotrachial intubation. Cumulus-oocyte-complexes (COCs) are recovered by aspiration of follicular contents under laparoscopic observation.

Recipient goats are fasted and an anesthetized in the same manner as the donors. A laparoscopic exploration is performed to confirm if the recipient has had one or more recent ovulations or corpora lutea present on the ovaries. An average of 11 nuclear transfer-derived embryos (1-cell to 4-cell stage) are transferred by means of a TomCat® catheter threaded into the oviduct ipsilateral to ovulation(s). Donors and recipients are monitored following surgical procedures and antibiotics and analgesics are administered according to approved procedures.

Oocyte maturation: COCs are cultured in 50 μl drops of maturation medium covered with an overlay of mineral oil and incubated at 38.5-39° C. in 5% CO2. The maturation medium consists of M199H (GIBCO) supplemented with bLH, bFSH, estradiol β-17, sodium pyruvate, kanamycin, cysteamine, and heat inactivated goat serum. After 23 to 24 hrs of maturation, the cumulus cells are removed from the matured oocytes by vortexing the COCs for 1-2 min in EmCare® containing hyaluronidase. The denuded oocytes are washed in handling medium (EmCare® supplemented with BSA) and returned to maturation medium. The enucleation process is initiated within 2 hr of oocyte denuding. Prior to enucleation, the oocytes are incubated in Hoechst 33342 handling medium for 20-30 minutes at 30-33° C. in air atmosphere.

Nuclear transfer: Oocytes are placed into manipulation drops (EmCare® supplemented with FBS) covered with an overlay of mineral oil. Oocytes stained with Hoechst are enucleated during a brief exposure of the cytoplasm to UV light (Zeiss Filter Set 01) to determine the location of the chromosomes. Stage of nuclear maturation is observed and recorded during the enucleation process.

The enucleated oocytes and dispersed donor cells are manipulated in handling medium. Donor cells are prepared by serum starving for 4 days at confluency. Subsequently they are trypsinized, rinsed once, and resuspended in Emcare® with serum. Small (<20 μm) donor cells with smooth plasma membranes are picked up with a manipulation pipette and slipped into perivitelline space of the enucleated oocyte. Cell-cytoplast couplets are fused immediately after cell transfer. Couplets are manually aligned between the electrodes of a 500 μm gap fusion chamber (BTX, San Diego, Calif.) overlaid with sorbitol fusion medium. A brief fusion pulse is administered by a BTX Electrocell Manipulator 200. After the couplets have been exposed to the fusion pulse, they are placed into 25 μl drops of medium overlaid with mineral oil. Fused couplets are incubated at 38.5-39° C. After 1 hr, couplets are observed for fusion. Couplets that have not fused are administered a second fusion pulse.

Oocyte activation and culture: Two to three hours after application of the first fusion pulse, the fused couplets are activated using calcium ionomycin and 6-dimethylaminopurine (DMAP) or using calcium ionomycin and cycloheximide/cytochalasin B treatment. Briefly, couplets are incubated for 5 minutes in EmCare® containing calcium ionomycin, and then for 5 minutes in EmCare® containing BSA. The activated couplets are cultured for 2.5 to 4 hrs in DMAP, then washed in handling medium and placed into culture drops (25 μl in volume) consisting of G1 medium supplemented with BSA under an oil overlay. Alternately, following calcium ionomycin treatment, the activated couplets are cultured for 5 hrs in cycloheximide and cytochalasin B, washed, and placed into culture. Embryos are cultured 12 to 18 hrs until embryo transfer. Nuclear transfer derived embryos are transferred on Day 1 (Day 0=day of fusion) into synchronized recipients on Day 1 of their cycle (Day 0=estrus).

Identification of transgenic goats: For confirmation of the presence of the transgene in nuclear transfer derived offspring, genomic DNA is extracted from the blood and ear biopsy of 2 week old kids using standard molecular biology techniques. The genomic DNA is isolated from the blood samples using a QIAamp® DNA Blood Mini Kit (Qiagen, Cat. # 51106), and from the tissue samples using DNeasy® Tissue Kit (Qiagen, cat #69506). For each sample, the DNA is eluted in 150-200 μl 0.1×AE buffer and stored at 4° C. until use.

The presence of the transgene in transgenic goats is confirmed by PCR, Southern hybridization, and FISH as described above for the stably transfected cell lines.

Induction of lactation and collection of milk: Transgenic female founders (F0 generation) are induced to lactate at 3-4 months of age in order to confirm the expression of recombinant hBMP-7 in milk. Induction of lactation and collection of milk are performed as described for recombinant hBMP-2 in Example 1, above.

Purification of BMP-7: The recombinant hBMP-7 protein is purified from skimmed milk of transgenic goats by heparin affinity chromatography as described in Example 1, above. Characterization of the purified recombinant hBMP-7 by electrophoresis under non-reducing conditions and be N-terminal sequence analysis is performed as described for recombinant hBMP-2 in Example 1, above.). A commercially available purified recombinant hBMP-7 (R&D Systems, Minneapolis, USA) is used as a standard for densiometric quantification.

BMP-7 ELISA assay: The level of recombinant hBMP-7 protein in skimmed milk of transgenic goats, and/or of purified recombinant hBMP-7 protein isolated from skimmed milk by heparin affinity chromatography, is quantitated using the BMP-7 Quantikine ELISA kit (R&D Systems, catalog #DY354) as per the manufacturers instructions.

In vitro BMP-7 activity assay: alkaline phosphatase induction in C2C12 cells: The activity of recombinant hBMP-7 protein is quantitated based upon induction of alkaline phosphatase in in vitro cultured C2C12 cells, performed as described for recombinant hBMP-2 in Example 1, above. Test BMP-7 samples included whey phase of skimmed milk from transgenic goats, purified recombinant hBMP-7 isolated from whey phase of skimmer milk from transgenic goats by heparin affinity chromatography, and as a positive control a commercially available purified recombinant hBMP-7 (R&D Systems, Minneapolis, USA).

In vivo BMP-7 activity assay: osteoinduction in mice: The osteoinductive capacity of recombinant hBMP-7 protein is measured using the mouse implantation model of osteoinduction, performed as described for recombinant hBMP-2 in Example 1, above.

### Results and Discussion

This example describes methods to generate the expression construct pBC1-GβCasSS-hBMP7. This expression construct is used to generate a linear GβCasSS-hBMP7 fragment used to generate stably transfected primary fetal goat cells, which are in turn used to generate transgenic goats via the nuclear transfer technique. The linear GβCasSS-hBMP7 fragment used to generate transgenic goats contains, in this order: 1) dimerized chicken β-globin gene insulator; 2) goat β-casein promoter; 3) goat β-casein exon 1, intron 1, and partial exon 2; 4) an XhoI cloning site; 5) β-casein signal sequence; 6) a hBMP-7 coding sequence and a STOP codon; 7) β-casein partial exon 7, intron 7, exon 8, intron 8 and exon 9; and 8) additional β-casein 3′ genomic sequence.

The presence of the hBMP-7 transgene in stably transfected cells lines and in founder (F0 generation) goats is confirmed by PCR and Southern blotting, and the presence of recombinant hBMP-7 in the milk of lactating goats is confirmed by ELISA. A permanent line of transgenic goats is established by breeding of the founder (F0) generation goats (either to non-transgenics goats, or by cross-breeding of an F0 male and F0 female).

Milk is collected from the transgenic goats. Recombinant hBMP-7 protein is purified from the collected milk by heparin affinity chromatography.

The biological activity of the recombinant hBMP-7 (either in crude form as the whey phase of skimmed milk from transgenics or in pure form following purification by heparin affinity chromatography) is verified and quantitated by both in vitro (alkaline phosphatase induction in C2C12 cells) and in vivo (osteoinduction in mice) techniques.

### Example 3

### Production of Recombinant BMP-2/BMP-7 Heterodimers in Transgenic Goats

### Materials and Methods

Generation of transgenic goats containing both the hBMP-2 and the h-BMP-7 transgene: A transgenic goat expressing recombinant hBMP-2 in mammary gland generated as described in Example 1 and a transgenic goat expressing recombinant hBMP-7 in mammary gland generated as described in Example 1 are mated to produce offspring that contain both the hBMP-2 and the hBMP-7 transgene.

The presence of both transgenes in the offspring of such a mating may be confirmed by the PCR, Southern hybridization, and FISH techniques described for the single transgene in Examples 1 and 2, above.

Induction of lactation and collection of milk: Transgenic goats are induced to lactate at 3-4 months of age in order to confirm the expression of recombinant hBMP-2 and hBMP-7 in milk. Induction of lactation and collection of milk are performed as described for recombinant hBMP-2 in Example 1, above.

Purification of BMP-2 homodimers, BMP-7 homodimers, and BMP-2/-7 heterodimers: The recombinant hBMP protein is purified from skimmed milk of transgenic goats by heparin affinity chromatography as described in Example 1, above. Characterization of the purified recombinant hBMP by electrophoresis under non-reducing conditions and be N-terminal sequence analysis is performed as described for recombinant hBMP-2 in Example 1, above.

Commercially available purified recombinant hBMP-2 and purified recombinant hBMP-7 (R&D Systems, Minneapolis, USA) are used as standards for densiometric quantification.

In particular, non-denaturing electrophoresis is used to quantitate the relative abundance of hBMP-2 homodimers, hBMP-7 homodimers, and hBMP-2/-7 heterodimers in the purified sample.

BMP ELISA assay: The level of recombinant hBMP-2 protein in skimmed milk of transgenic goats, and/or of purified recombinant hBMP-2 protein isolated from skimmed milk by heparin affinity chromatography, is quantitated using the BMP-2 Quantikine ELISA kit (R&D Systems, catalog # DBP200) as per the manufacturer's instructions. The level of recombinant hBMP-7 protein in skimmed milk of transgenic goats, and/or of purified recombinant hBMP-7 protein isolated from skimmed milk by heparin affinity chromatography, is quantitated using the BMP-7 Quantikine ELISA kit (R&D Systems, catalog #DY354) as per the manufacturer's instructions.

In vitro BMP activity assay: alkaline phosphatase induction in C2C12 cells: The activity of hBMP-2 homodimers, hBMP-7 homodimers, and hBMP-2/-7 heterodimers is quantitated based upon induction of alkaline phosphatase in in vitro cultured C2C12 cells, performed as described for recombinant hBMP-2 in Example 1, above. Test BMP samples included whey phase of skimmed milk from transgenic goats, purified recombinant hBMP-2 homodimers, hBMP-7 homodimers, and hBMP-2/-7 heterodimers isolated from whey phase of skimmed milk from transgenic goats by heparin affinity chromatography, and as a positive control commercially available purified recombinant hBMP-2 and recombinant hBMP-7 (R&D Systems, Minneapolis, USA).

In vivo BMP activity assay: osteoinduction in mice: The osteoinductive capacity of recombinant hBMP-2 homodimers, hBMP-7 homodimers, and hBMP-2/-7 heterodimers is measured using the mouse implantation model of osteoinduction, performed as described for recombinant hBMP-2 in Example 1, above

### Results and Discussion

This example describes methods to generate transgenic goats harboring both an hBMP-2 transgene and an hBMP-7 transgene, and which therefore express recombinant hBMP-2 and hBMP7 in the mammary gland.

A permanent line of such doubly transgene goats is established by breeding of the hBMP-2 transgenic goats of Example 1 to the hBMP-7 transgenic goats of Example 2.

Milk is collected from these transgenic goats. Recombinant hBMP-2 homodimers, hBMP-7 homodimers, and hBMP-2/-7 heterodimers are purified from the collected milk by heparin affinity chromatography.

The biological activity of the recombinant hBMP-2 homodimers, hBMP-7 homodimers, and hBMP-2/-7 heterodimers (either in crude form as the whey phase of skimmed milk from transgenics or in pure form following purification by heparin affinity chromatography) is verified and quantitated by both in vitro (alkaline phosphatase induction in C2C12 cells) and in vivo (osteoinduction in mice) techniques.

### Example 4

### In Vitro Cell Based Assay to Measure the Activity of BMP

### Materials and Methods

Preparation of test materials: Recombinant human BMP-2 (rhBMP-2), recombinant human BMP-4 (rhBMP-4) and recombinant human BMP-7 (rhBMP-7) all made in CHO cells was purchased from R&D Systems Inc. (Minneapolis, Min.). Recombinant human BMP-2 and rhBMP-7 were also produced in CHO cultures in house. All BMP preparations were resuspended in 4 mM HCl+0.1% BSA.

BMP activity assay: C2C12 cells (ATCC; Manassas, Va.) were cultured in alpha Minimal Essential Medium (αMEM: InVitrogen, Burlington CANADA) containing 10% heat inactivated fetal bovine serum (FBS: InVitrogen) and were maintained at 37° C. in 5% CO₂.

Cultures were passaged before confluence and resuspended at 0.5×10⁵ cells/ml in αMEM+15% FBS supplemented with 50 μg/ml ascorbic acid (Sigma, St Louis Mo.) (assay medium). One milliliter of cell suspension was seeded into each well of a 24 well tissue culture plate.

After 4 to 24 hours the medium is changed to assay medium plus the test material. After a further 48 to 72 hours the cultures were terminated.

At time of harvest cultures were rinsed three times with Tris buffered saline (TBS; 20 mM Tris, 137 mM NaCl, pH 7.4). Lysis buffer (CelLytic, Sigma) was added to each well, and the cell layer scraped into Epindorf tubes and sonicated. The lysate was centrifuged at 5,000 g at 4 ° C. for 10 minutes and the supernatant assayed for alkaline phosphatase (Sigma protocol 104) and protein content (Coomasie Plus; Pierce Chemical Co., Rockford Ill.). Standard curves were generated using p-nitrophenol standard solution in 0.02 N NaOH for the AP assay and bovine serum albumin in lysis buffer for the protein assay.

The BMP activity of the test material was determined by calculating the alkaline phosphatase activity expressed per well or normalized to protein content.

Statistical Analysis: For any given experiment each data point represents the mean±SD of three to eight individual cultures. Statistics were performed by analysis of variance (ANOVA) folled by a post-hoc test to determine the significance between groups. A P-value of less than 0.05 was considered significant.

### Results and Discussion

Recombinant human BMP-2 rhBMP-4 and rhBMP-7 demonstrated dose dependant increases in ALP activty in C2C12 cultures (Table 3). We also noted that rhBMP-7 had significantly lower biological activity in this assay than rhBMP-2 or rhBMP-4 (Table 4).

TABLE 3
Dose response curve for stimulation of alkaline phosphatase (ALP)
activity in C2C12 cultures treated with rhBMP-2.

| | ALP (U/ug protein) |
|---|---|
| Dose (ng/ml) | Mean ± SD |
| 0 | 2.9 ± 0.9 |
| 10 | 3.4 ± 0.7 |
| 20 | 6.1 ± 2.2 |
| 40 | 9.4 ± 2.4 |
| 80 | 17.4 ± 3.6 |

TABLE 4
Comparison of the biological activity of different
BMPs using the C2C12 in vitro assay.

| | ALP (U/ug protein) |
|---|---|
| Group | Mean ± SD |
| Control | 4.7 ± 0.3 |
| rhBMP-2 (20 ng/ml) | 10.6 ± 2.6 |
| rhBMP-4 (20 ng/ml) | 8.5 ± 0.9 |
| rhBMP-7 (20 ng/ml) | 5.8 ± 0.6 |

### Example 5

### In Vitvo Asssya for the Testing of BMP Activity

### Materials and Methods

Preparation of test materials Partially purified BMP/NCP was prepared by the method of Urist et al. 1987 Methods Enzymol. 1987; 146:294-312. Briefly, 5 Kg of cleaned bovine cortical bone was frozen in liquid nitrogen, ground to approximately 1 mm³, defatted overnight in chloroform-methanol (1:1) and decalcified in 0.6N HCl for 72 hours. The demineralized bone matrix was extracted with 6M urea/0.5M CaCl₂ (urea-CaCl₂) and the extract dialyzed against distilled water. The precipitate which formed was redissolved into urea-CaCl₂ and dialyzed against 0.25M citric acid. The precipitate was defatted in chloroform-methanol (1:1), resupended in urea-CaCl2, and dialyzed against 6M urea in 0.1M Tris, 0.2% Triton X-100 (pH 7.2), followed by dialysis against distilled water. The precipitate was collected, lyophilized and designated as BMP/NCP. Recombinant human BMP-7 combined with collagen partciles was purchased from Stryker Biotech. The purified and recombinant BMPs were placed into gelatin capsules (1 to 10 mg per capsule) and the capsules sterilized over chloroform vapours.

In Vivo Assay for BMP activity: Gelatin capsules containing BMP were implanted bilaterally into the thigh muscles of Swiss Webster mice (n=4 per group). Each implant was harvested 4 weeks post-implantation. The hind limbs were placed on the x-ray film, which was then exposed at 30 kV, 4 mA for 30 seconds.

Micro-computed tomographic scanner (GE Health Care, London, Ontario) was used to scan the left and the right legs. X-ray energy setting of 80 kV and 80 μA were applied to the sample over one full 360° rotation. The scanner produced a 2-dimentional (2D) projection images in 1.11° angular increments around the object resulting in 400 views. Bright field (an x-ray projection with no object in the field of view) and dark field (an image acquired without any x-rays) were collected for correction of the acquisition images. The 2D projections were reconstructed into a 3D volume. From this 3D volume the induced bone was identified and the bone mineral content and volumetric bone mineral densirty calculated with the assistance of the software (GE Healthcare eXplore MicroView v. 2.0) provided with the scanner.

The hind limbs were fixed in 10% neutral formalin and decalcified in 45% formic acid in 20% sodium citrate. The decalcified hind limbs were embedded in wax, sectioned perpendicularly to the thigh bone axis, and stained with hematoxylin and eosin.

### Results and Discussion

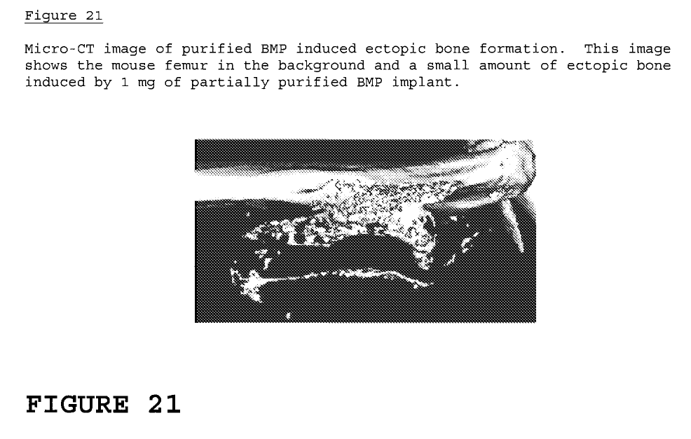

The volume of bone generated by the OP-1 implants was significantly larger than that generated by the purified BMP. Use of the micro-CT enabled identification and measurement of small amounts of induced ectopic bone (FIG. 20). Histological analysis of these confirmed that they were composed of bone surrounding a marrow cavity (FIG. 21).

### Example 6

### Inactivation of Biological Activity of BMP by Retention of Pro Sequence

### Materials and Methods

Preparation of test materials: Recombinant human BMP-2 (rhBMP-2) made in CHO cells was purchased from R&D Systems Inc. (Minneapolis, Minn.). Recombinant human ProBMP-MP-2 and rhBMP-2 both produced in E-coli was purchased from (Cedarlane, Hornsby, CANADA). preparations were resuspended in 4 mM HCl+0.1% BSA. To ensure that ProBMP was not converted into the mature protein when added to the cell cultures the furin inhibitor 9DR (custom peptide synthesized by Advanced SynTech Corporation, Markham ON) was added to some cultures.

Asessment of activity: The biological activity of each test material was determined using the assay described in example 4.

### Results and Discussion

ProBMP-2 in the presence or absence of 9DR failed to stimulate alkaline phosphatase activity at either dose tested, while the mature rhBMP-2 made in E-coli possessed low but signifacant activity (Table 5).

TABLE 5
Activity of Pro-BMP-2 and mature BMP-2 in vitro.

| | | | | BMP-2 | BMP-2 | ALP |
|---|---|---|---|---|---|---|
| | | 9DR | ProBMP | (CHO) | (Ecoli) | Units |
| Group | n | (uM) | (nM) | (nM) | (nM) | (mean ± SD) |
| 1 | 8 | | | | | 1.38 ± 0.23 |
| 2 | 4 | 1 | | | | 0.78 ± 0.15 |
| 3 | 4 | | 50 | | | 1.23 ± 0.21 |
| 4 | 4 | | | 50 | | 39.13 ± 3.81 |
| 5 | 4 | | | | 50 | 2.00 ± 0.30 |
| 6 | 4 | 1 | 50 | | | 1.01 ± 0.10 |
| 7 | 4 | 1 | | 50 | | 37.31 ± 0.94 |
| 8 | 4 | 1 | | | 50 | 1.44 ± 0.23 |
| 9 | 4 | | 100 | | | 1.24 ± 0.13 |
| 10 | 4 | | | 100 | | 95.98 ± 15.88 |
| 11 | 4 | | | | 100 | 2.76 ± 0.43 |
| 12 | 4 | 1 | 100 | | | 1.43 ± 0.59 |
| 13 | 4 | 1 | | 100 | | 85.88 ± 14.19 |
| 14 | 4 | 1 | | | 100 | 3.99 ± 0.00 |

### Example 7

### Inhibition of Osteoinductive Activity of RHBMP-2 by Noggin

### Materials and Methods

Peperation of test materials: CHO cell produced rhBMP-2 and noggin were obtained from R&D Systems (Minneapolis, USA). Test materials containing either nothing, rhBMP-2, Noggin or rhBMP-2+noggin at various concentrations were prepared.

Assesment of activity: Test materials were assayed for BMP activity as described in Example 4.

### Results and Discussion

The addition of noggin reduced the activity of rhBMP-2 in the C2C12 asay. One nano-mole of noggin partially inhibited 50 ng/ml of rhBMP-2 while 3 nM and above complety inhibited rHBMP-2 activity (Table 6).

TABLE 6
Inhibition of rhBMP-2 activity by rmNoggin.

| | | rhBMP-2 | Noggin | ALP (U/ug protein) |
|---|---|---|---|---|
| Group | n | (ng/ml) | (nM) | (mean ± SD) |
| 1 | 4 | — | — | 5.8 ± 0.3 |
| 2 | 4 | 50 | — | 38.1 ± 2.6 |
| 3 | 4 | 50 | 1 | 10.4 ± 1.9 |
| 4 | 4 | 50 | 3 | 3.1 ± 0.5 |
| 5 | 4 | 50 | 4 | 3.7 ± 0.9 |
| 6 | 4 | 50 | 5 | 3.8 ± 1.0 |

### Example 8

### Resistance of RHBMP Biological Activity to Prescission digestion

To determine whether enzyme digestion can be used to activate transgenic rhBMP-2 we tested the bioloical activity of rhBMP-2 after treatment with PreScission protease.

### Materials and Methods

Preparation of test materials: PreScisssion protease was obtained from Amersham Bioscinces (GE Healthcare, Buckinghamshire, U.K asn was prepared according to manufactors instructions. Recombinant hBMP-2 and rhBMP-7 (CHO cell produced) were R&D Systems. The rhBMP samples were resuspended in 4 mM HCl to a final concentration of 10 μg/ml. BSA was not included so that the only substrate for the protease was BMP. The clevage buffer was prepared (50 mM Tris-HCl, 150 mM NaCl, 1 mM EDTA, pH 7).

Assesment of activity: Samples underwent digestion at room temperature for 6 hours. Reactions were stopped by freezing at −20° C. Digestion samples were diluted in αMEMS+15% FBS and tested for BMP activity using the C2C12 assay described in example 4.

### Results and Discussion

The Results demonstrated that both rhBMP-2 and rhBMP-7 treated with PreScissionremained active after digestion at room temperature for 6 hours (Table 7 & 8). No loss of activity was seen in comparison to control BMP incubated in cleavage buffer alone, althought there was a 30 to 30% decline in activity compared to unincubated BMP (Table 7 & 8).

TABLE 7
Effect of PrecScission protease digestion on rhBMP-2.

| | | rhBMP-2 | Precission | Cleavage | ALP activity |
|---|---|---|---|---|---|
| | | (10 ug/ml) | Protease | Buffer | (U/ug protein) |
| Group | n | (ul) | (ul) | (ul) | mean ± SD |
| 1 | 4 | — | — | — | 2.6 ± 0.5 |
| 2 | 4 | 20 | — | — | 19.0 ± 2.0 |
| 3 | 4 | 20 | 15 | 165 | 12.5 ± 1.0 |
| 4 | 4 | 20 | — | 180 | 12.0 ± 0.5 |
| 5 | 4 | — | 15 | 185 | 2.0 ± 0.5 |

TABLE 8
Effect of PreScission protease digestion on rhBMP-7.

| | | rhBMP-7 | Precission | Cleavage | ALP activity |
|---|---|---|---|---|---|
| | | (10 ug/ml) | Protease | Buffer | (U/ug protein) |
| Group | n | (ul) | (ul) | (ul) | mean ± SD |
| 1 | 4 | — | — | — | 1.6 ± 0.2 |
| 2 | 4 | 20 | — | — | 7.1 ± 0.5 |
| 3 | 4 | 20 | 15 | 165 | 2.9 ± 0.2 |
| 4 | 4 | 20 | — | 180 | 4.0 ± 0.3 |
| 5 | 4 | — | 15 | 185 | 1.5 ± 0.1 |

### Example 9

### Measuring the Amount of BMP Present in Milk

To measure the amount of BMP in various solutions we determine the ability of an ELISA to measure BMP in buffer and goats milk. We also investigated the effect of incubating rhBMP-2 with noggin on the ability to measure concentration of rhBMP present in milk.

### Materials and Methods

Preparation of test materials: Recombinant human BMP-2 was prepared as described in example 4. An ELISA for the measurement of rhBMP-2 was purchased from R&D Sytems (catalogue number DBP 200). Standards were prepared by diluting rhBMP-2 in goats milk or in the calibration buffer. The ELISA was performed as per the manufacturers instructions.

### Results and Discussion

We found that the absorbance measurements were reduced at each concentration of rhBMP-2 in milk compared to the same concentration of rhBMP-2 in calibration buffer (Table 9). However the increase in absorbance was proportional to the concentrations of rhBMP-2 spiked into the milk (Table 9). Using the calibration curve generated with rhBMP-2 spiked into milk we estimate that the ELISA has a lower limit of detection of 0.25 ng/ml milk and a lower limit of quantitation between 0.5 and 1.0 ng/ml milk.

TABLE 9
Measurement of rhBMP-2 in goats milk.

| | Absorbance Readings | | |
|---|---|---|---|
| | (Arbitary units) | | |
| rhBMP-2 | | | Milk |
| (ng/ml) | Calibration buffer | Milk | (corrected to zero) |
| 0.00 | 0.006 ± 0.010 | −0.018 ± 0.001 | 0.000 |
| 0.13 | 0.003 ± 0.001 | −0.012 ± 0.000 | 0.006 |
| 0.25 | 0.007 ± 0.001 | −0.008 ± 0.003 | 0.010 |
| 0.50 | 0.034 ± 0.004 | 0.003 ± 0.004 | 0.021 |
| 1.00 | 0.132 ± 0.003 | 0.046 ± 0.006 | 0.064 |
| 2.00 | 0.432 ± 0.004 | 0.192 ± 0.001 | 0.209 |
| 4.00 | 1.365 ± 0.058 | 0.656 ± 0.033 | 0.673 |

### Example 10

### Purification of BMP+Noggin from Milk Using Heparin-Sepharose Chromatography

To determine the ferasibility of using Heparin affinity chromatography to purify BMP or BMP-Noggin complexes from from milk.

### Materials and Methods

Preparation of test materials: Recombinant human BMP-2+noggin was prepared by adding 200 ng/ml rhBMP to 600 ng/ml rmNoggin. After 1 hour the BMP-Noggin mixture was added to 5 mls of goat milk.

Purification of BMP and BMP-Noggin from milk using Heparin affinity chromatography. Heparin-Agarose pre-packed columns were purchased from Sigma (catalogue # Hep-I-5) and prepared as described in the manufacturers instructions. The heparin-agarose column was pre-equilibrated with 13 column volumes of 100 mM Tris-HCl buffer at pH 8 at 4° C.

The milk containing the rhBMP-2-rmNoggin was mixed with 8 ml 100 mM Tris-HCl pH 8 and the volume reduced to 9.5 mls by loading onto an Amicon ultra-15 PLCC (UFC9 005 08) centrifugal filter unit, MWCO 5 k Da (Millipore, Billercia Mass.) and centrifugeing at 4000 g for 15 minutes at 4° C.

The column was loaded with the sample and then the column was washed with 9 column volumes of buffer to remove unbound contaminating proteins. The flow through was collected for analysis by ELISA.

The bound proteins were eluted isocratically using 1.8 column volumes of elution buffer, 100 mM Tris-HCl buffer at pH 8 containing incresing amounts of NaCl. The amounts of NaCl were 100, 200, 300, 500, 600 mM and 1000 mM. All elutions were carried out undergravity at 4° C. Each fraction was collected and the amount of BMP-2 on was assayed by ELISA as described in Example 9.

### Results and Discussion

The results indicated that virtually complete recovery of the BMP could be achieved from the herparin sepharose column, even in the presence of Noggin (Table 10).

TABLE 10
Purification of BMP from milk using Heparin affinity chromatography.

| | BMP | |
|---|---|---|
| | eluted | Percent |
| ELISA results | (ng) | Recovery |
| Starting Sample | 20.52 | 100% |
| 100 Mm | 0.00 | 0.0% |
| 200 Mm | 0.00 | 0.0% |
| 300 mM | 0.00 | 0.0% |
| 500 mM | 1.60 | 4.9% |
| 600 mM | 1.86 | 9.1% |
| 1000 mM | 21.41 | 104.3% |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A non-human transgenic mammal that upon lactation, expresses a recombinant BMP in its milk, wherein the genome of the mammal comprises a nucleic acid sequence encoding a recombinant BMP, optionally a nucleic acid sequence encoding a recombinant BMP-inhibitor, both operably linked to a mammary gland-specific promoter, and a signal sequence that provides secretion of the recombinant BMP and BMP-inhibitor into the milk of the mammal.

2. The transgenic mammal of claim 1 wherein the mammary gland-specific promoter is a casein promoter.

3. The transgenic mammal of claim 1, wherein the mammal is a goat.

4. The transgenic mammal of claim 1, wherein the recombinant BMP is a recombinant human BMP.

5. The transgenic mammal of claim 1, wherein the recombinant BMP is a recombinant BMP-2 or a recombinant BMP-7.

6. The transgenic mammal of claim 1, wherein the recombinant BMP-inhibitor is a recombinant human BMP-inhibitor.

7. The transgenic mammal of claim 1, wherein the recombinant BMP-inhibitor is a recombinant Noggin, Chordin, Sclerostin or Gremlin.

8. The transgenic mammal of claim 1, wherein the recombinant BMP is a recombinant furin-resistant mutant BMP.

9. The transgenic mammal of claim 1, wherein the recombinant BMP is a recombinant furin-resistant mutant BMP-2 or recombinant furin-resistant mutant BMP-7.

10. The transgenic mammal of claim 1, wherein the recombinant BMP is a recombinant BMP/BMP-inhibitor fusion protein.

11. The transgenic mammal of claim 1, wherein the recombinant BMP is a recombinant BMP/BMP-inhibitor fusion protein comprising BMP-2 and Noggin.

12. The transgenic mammal of claim 1, wherein the recombinant BMP is a recombinant BMP/BMP-inhibitor fusion protein comprising BMP-7 and Sclerostin.

13. A genetically-engineered nucleic acid sequence, which comprises: (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) at least one signal sequence that provides secretion of the recombinant BMP and BMP-inhibitor.

14. The genetically-engineered nucleic acid sequence of claim 13, wherein the mammary gland-specific promoter is a casein promoter.

15. A mammalian cell which has been transformed to comprise the nucleic acid sequence of claim 13.

16. The mammalian cell of claim 15, wherein the cell is selected from the group of embryonic stem cells, embryonal carcinoma cells, primordial germ cells, oocytes, and sperm.

17. The mammalian cell of claim 15, wherein the cell is a primary fetal goat cell.

18. The mammalian cell of claim 15, wherein the cell is a mammary epithelium cell line.

19. A non-human mammalian embryo, into which has been introduced the genetically-engineered nucleic acid sequence of claim 13.

20. The genetically-engineered nucleic acid sequence of claim 13, wherein the recombinant BMP is a recombinant human BMP.

21. The genetically-engineered nucleic acid sequence of claim 13, wherein the recombinant BMP is a recombinant BMP-2 or a recombinant BMP-7.

22. The genetically-engineered nucleic acid sequence of claim 13, wherein the recombinant BMP-inhibitor is a recombinant human BMP-inhibitor.

23. The genetically-engineered nucleic acid sequence of claim 13, wherein the recombinant BMP-inhibitor is a recombinant Noggin, Chordin, Sclerostin or Gremlin.

24. The genetically-engineered nucleic acid sequence of claim 13, wherein the recombinant BMP is a recombinant furin-resistant mutant BMP.

25. The genetically-engineered nucleic acid sequence of claim 13, wherein the recombinant BMP is a recombinant furin-resistant mutant BMP-2 or a recombinant furin-resistant mutant BMP-7.

26. The genetically-engineered nucleic acid sequence of claim 13, wherein the recombinant BMP is a recombinant BMP/BMP-inhibitor fusion protein.

27. The genetically-engineered nucleic acid sequence of claim 13, wherein the recombinant BMP is a recombinant BMP/BMP-inhibitor fusion protein comprising BMP-2 and Noggin.

28. The genetically-engineered nucleic acid sequence of claim 13, wherein the recombinant BMP is a recombinant BMP/BMP-inhibitor fusion protein comprising BMP-7 and Sclerostin.

29. A method for making a genetically-engineered nucleic acid sequence, which method comprises joining a nucleic acid sequence encoding a recombinant BMP, and optionally a nucleic acid sequence encoding a recombinant BMP-inhibitor, with at least one mammary gland-specific promoter that directs expression of the recombinant BMP and BMP-inhibitor, and with at least one signal sequence that provides secretion of the recombinant BMP and BMP-inhibitor.

30. The method of claim 29, wherein the recombinant BMP is a recombinant furin-resistant mutant BMP.

31. The method of claim 29, wherein the recombinant BMP is a recombinant BMP/BMP-inhibitor fusion protein.

32. A method for producing a transgenic non-human mammal that upon lactation secretes a recombinant BMP in its milk, which method comprises allowing an embryo, into which has been introduced a genetically-engineered nucleic acid sequence, comprising (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) at least one signal sequence that provides secretion of the recombinant BMP and BMP-inhibitor into the milk of the mammal, to grow when transferred into a recipient female mammal, resulting in the recipient female mammal giving birth to the transgenic mammal.

33. The method of claim 32, wherein the mammary gland-specific promoter is a casein promoter.

34. The method of claim 32, wherein the embryo is a goat embryo.

35. The method of claim 32, wherein the recombinant BMP is a recombinant human BMP.

36. The method of claim 32, wherein the recombinant BMP is a recombinant BMP-2 or a recombinant BMP-7.

37. The method of claim 32, wherein the recombinant BMP is a recombinant furin-resistant mutant BMP.

38. The method of claim 32, wherein the recombinant BMP is a recombinant furin-resistant mutant BMP-2 or a recombinant furin-resistant mutant BMP-7.

39. The transgenic mammal of claim 32, wherein the recombinant BMP is a recombinant BMP/BMP-inhibitor fusion protein.

40. The transgenic mammal of claim 32, wherein the recombinant BMP is a recombinant BMP/BMP-inhibitor fusion protein comprising BMP-2 and Noggin.

41. The transgenic mammal of claim 32, wherein the recombinant BMP is a recombinant BMP/BMP-inhibitor fusion protein comprising BMP-7 and Sclerostin.

42. The method of claim 32, which further comprises introducing the genetically-engineered nucleic acid sequence into a cell of the embryo, or into a cell that will form at least part of the embryo.

43. The method of claim 42, wherein introducing the genetically-engineered nucleic acid sequence comprises pronuclear or cytoplasmic microinjection of the genetically-engineered nucleic acid sequence.

44. The method of claim 42, wherein introducing the genetically-engineered nucleic acid sequence comprises combining a mammalian cell stably transfected with the genetically-engineered nucleic acid sequence with a non-transgenic mammalian embryo.

45. The method of claim 42, wherein introducing the genetically-engineered nucleic acid sequence comprises the steps of
(a) introducing the genetically-engineered nucleic acid sequence into a non-human mammalian oocyte; and
(b) activating the oocyte to develop into an embryo.

46. A method for producing a non-human transgenic mammal that upon lactation secretes a recombinant BMP in its milk, which method comprises breeding or cloning a transgenic mammal, the genome of which comprises a genetically-engineered nucleic acid sequence, comprising (i) a nucleic acid sequence encoding a recombinant BMP; (ii) optionally a nucleic acid sequence encoding a recombinant BMP-inhibitor; (iii) at least one mammary gland-specific promoter that directs expression of the recombinant BMP and BMP-inhibitor; and (iv) at least one signal sequence that provides secretion of the recombinant BMP and BMP-inhibitor into the milk of the mammal.

47. The method of claim 46, wherein the recombinant BMP is a recombinant furin-resistant mutant BMP.

48. The method of claim 46, wherein the recombinant BMP is a recombinant BMP/BMP-inhibitor fusion protein.

49. A method for producing a recombinant BMP, which method comprises:
(a) inducing or maintaining lactation of a transgenic mammal, the genome of which comprises a nucleic acid sequence encoding a recombinant BMP, optionally a recombinant BMP-inhibitor, both operably linked to a mammary gland-specific promoter, wherein the sequence further comprises a signal sequence that provides secretion of the recombinant BMP and BMP-inhibitor into the milk of the mammal; and
(b) extracting milk from the lactating mammal.

50. The method according to claim 49, which comprises the additional steps of:
(a) optional proteolytic cleavage of the recombinant BMP; and
(b) purifying the recombinant BMP from the extracted milk.

51. The milk of a non-human mammal comprising a recombinant BMP.

52. The milk of claim 51, where the milk is whole milk.

53. The milk of claim 51, where the milk is defatted milk.

54. A method for producing a recombinant BMP in a culture of mammary epithelium cells, which method comprises:
(a) culturing said cells, into which a nucleic acid sequence comprising (i) a nucleic acid sequence encoding a recombinant BMP, (ii) a mammary gland-specific promoter that directs expression of the recombinant BMP within said cells, and (iii) a signal sequence that provides secretion of the recombinant BMP into the cell culture medium, has been introduced;
(b) culturing the cells; and
(c) collecting the cell culture medium of the cell culture.

55. The method of claim 54, which comprises the additional steps of:
(a) optional proteolytic cleavage of the recombinant BMP; and
(b) purifying the recombinant BMP from the collected cell culture medium.

56. The method of claim 54, wherein the mammary epithelium cells are MAC-T cells (ATCC Number CRL 10274).

57. The method of claim 54, wherein the mammary epithelium cells are 184B5 cells (ATCC Number CRL-8799), 184A1 cells (ATCC Number CRL-8798), MCF7 cells (ATCC Number HTB-22), or ZR-75-30 cells (ATCC Number CRL-1504).

58. Cell culture medium comprising a recombinant BMP produced by cultured mammary epithelium cells.

59. A protein comprising a recombinant BMP containing:
(a) a mutated furin proteolytic cleavage sequence such that the protein is resistant to proteolytic cleavage by furin or furin-like proteases; and
(b) a non-furin proteolytic cleavage sequence such that the protein is susceptible to proteolytic cleavage.

60. A protein according to claim 59, wherein said protein does not have BMP activity.

61. A protein according to claim 59, wherein said protein has BMP activity after proteolytic cleavage.

62. A protein according to claim 59, wherein said protein is recombinant BMP-2, recombinant BMP-4 or recombinant BMP-7, or a homodimer or heterodimer thereof.

63. A protein according to claim 59, wherein said protein is a recombinant human BMP-2, recombinant human BMP-4 or recombinant human BMP-7, or a homodimer or heterodimer thereof.

64. A fusion protein comprising a recombinant BMP, a recombinant BMP-inhibitor and a linker region containing at least one proteolytic cleavage site.

65. A fusion protein according to claim 64, wherein said protein does not have BMP activity.

66. A fusion protein according to claim 64, wherein said protein has BMP activity after proteolytic cleavage.

67. A fusion protein according to claim 64, wherein said recombinant BMP is recombinant BMP-2, and said recombinant BMP-inhibitor is a recombinant Noggin.

68. A fusion protein according to claim 64, wherein said recombinant BMP is recombinant BMP-7, and said recombinant BMP-inhibitor is a recombinant Sclerostin.

69. A fusion protein according to claim 64, wherein said recombinant BMP is a recombinant human BMP-2, and said recombinant BMP-inhibitor is a recombinant human Noggin.

70. A fusion protein according to claim 64, wherein said recombinant BMP is a recombinant human BMP-7, and said recombinant BMP-inhibitor is a recombinant human Sclerostin.

71. A method for producing a pharmaceutical composition, which comprises combining
(a) a recombinant BMP produced by a transgenic mammal according to the method of claim 49 with
(b) a pharmaceutically acceptable carrier or excipient.

72. A method for producing a pharmaceutical composition, which comprises combining
(a) a recombinant BMP produced in a culture of mammary epithelium cells according to the method of claim 54 with
(b) a pharmaceutically acceptable carrier or excipient.

73. A non-human transgenic mammal that upon lactation, expresses a recombinant BMP in its milk, wherein the genome of the mammal comprises (a) a first nucleic acid sequence encoding a first recombinant BMP, operably linked to a first mammary gland-specific promoter, and a first signal sequence that provides secretion of the first recombinant BMP into the milk of the mammal; (b) a second nucleic acid sequence encoding a second recombinant BMP, operably linked to a second mammary gland-specific promoter, and a second signal sequence that provides secretion of the second recombinant BMP into the milk of the mammal; and (c) optionally a third nucleic acid sequence encoding a recombinant BMP-inhibitor, operably linked to a third mammary gland-specific promoter, and a third signal sequence that provides secretion of the recombinant BMP-inhibitor into the milk of the mammal.

74. The transgenic mammal of claim 73 wherein the first mammary gland-specific promoter, the second mammary gland-specific promoter and the third mammary gland-specific promoter are casein promoters.

75. The transgenic mammal of claim 73, wherein the mammal is a goat.

76. The transgenic mammal of claim 73, wherein the first recombinant BMP is a recombinant human BMP, the second recombinant BMP is a recombinant human BMP and the recombinant BMP-inhibitor is a recombinant human BMP-inhibitor.

77. The transgenic mammal of claim 73, wherein the first recombinant BMP is a recombinant BMP-2, the second recombinant BMP is a recombinant BMP-7 and the recombinant BMP-inhibitor is a recombinant Gremlin.

78. A method for producing a non-human transgenic mammal that upon lactation secretes a recombinant BMP in its milk, which method comprises allowing an embryo, into which has been introduced a first genetically-engineered nucleic acid sequence, a second genetically-engineered nucleic acid sequence and optionally a third genetically-engineered nucleic acid sequence, to grow when transferred into a recipient female mammal, resulting in the recipient female mammal giving birth to the transgenic mammal,
wherein the first genetically-engineered nucleic acid sequence comprises (i) a first nucleic acid sequence encoding a first recombinant BMP; (ii) a first mammary gland-specific promoter that directs expression of the first recombinant BMP; and (iii) a first signal sequence that provides secretion of the first recombinant BMP into the milk of the mammal, and
wherein the second genetically-engineered nucleic acid sequence comprises (i) a second nucleic acid sequence encoding a second recombinant BMP; (ii) a second mammary gland-specific promoter that directs expression of the second recombinant BMP; and (iii) a second signal sequence that provides secretion of the second recombinant BMP into the milk of the mammal, and
wherein the third genetically-engineered nucleic acid sequence comprises (i) a third nucleic acid sequence encoding a recombinant BMP-inhibitor; (ii) a third mammary gland-specific promoter that directs expression of the recombinant BMP-inhibitor; and (iii) a third signal sequence that provides secretion of the recombinant BMP-inhibitor into the milk of the mammal.

79. The method of claim 78, wherein the first mammary gland-specific promoter, the second mammary gland-specific promoter and the third mammary gland-specific promoter are casein promoters.

80. The method of claim 78, wherein the embryo is a goat embryo.

81. The method of claim 78, wherein the first recombinant BMP is a recombinant human BMP, the second recombinant BMP is a recombinant human BMP and the recombinant BMP-inhibitor is a recombinant human BMP-inhibitor.

82. The method of claim 78, wherein the first recombinant BMP is a recombinant BMP-2, the second recombinant BMP is a recombinant BMP-7 and the recombinant BMP-inhibitor is a recombinant Gremlin.

83. The method of claim 78, which further comprises introducing the first, second or third genetically-engineered nucleic acid sequence into a cell of the embryo, or into a cell that will form at least part of the embryo.

84. The method of claim 83, which further comprises introducing the first genetically-engineered nucleic acid sequence into a cell of the embryo, or into a cell that will form at least part of the embryo, introducing the second genetically-engineered nucleic acid sequence into a cell of the embryo, or into a cell that will form at least part of the embryo and introducing the third genetically-engineered nucleic acid sequence into a cell of the embryo, or into a cell that will form at least part of the embryo.

85. The method of claim 83, wherein introducing the first, second or third genetically-engineered nucleic acid sequence comprises pronuclear or cytoplasmic microinjection of the first, second or third genetically-engineered nucleic acid sequence.

86. The method of claim 83, wherein introducing the first, second or third genetically-engineered nucleic acid sequence comprises combining a mammalian cell stably transfected with the first, second or third genetically-engineered nucleic acid sequence with a non-transgenic mammalian embryo.

87. The method of claim 83, wherein introducing the first, second or third genetically-engineered nucleic acid sequence comprises the steps of
(a) introducing the first, second or third genetically-engineered nucleic acid sequence into a non-human mammalian oocyte; and
(b) activating the oocyte to develop into an embryo.

88. A method for producing a non-human transgenic mammal that upon lactation secretes a recombinant BMP in its milk, which method comprises breeding a first transgenic mammal, the genome of which comprises a first genetically-engineered nucleic acid sequence, comprising (i) a first nucleic acid sequence encoding a first recombinant BMP; (ii) a first mammary gland-specific promoter that directs expression of the first recombinant BMP; and (iii) a first signal sequence that provides secretion of the first recombinant BMP into the milk of the mammal, to a second transgenic mammal, the genome of which comprises a second genetically-engineered nucleic acid sequence, comprising (i) a second nucleic acid sequence encoding a second recombinant BMP; (ii) a second mammary gland-specific promoter that directs expression of the second recombinant BMP; and (iii) a second signal sequence that provides secretion of the second recombinant BMP into the milk of the mammal.

89. The method of claim 88, wherein the first mammary gland-specific promoter and the second mammary gland-specific promoter are casein promoters.

90. The method of claim 88, wherein the first transgenic animal and the second transgenic animal are goats.

91. The method of claim 88, wherein the first recombinant BMP and the second recombinant BMP are recombinant human BMPs.

92. The method of claim 88, wherein the first recombinant BMP is a recombinant BMP-2 and the second recombinant BMP is a recombinant BMP-7.

93. The method of claim 88, wherein the first recombinant BMP and the second recombinant BMP are recombinant furin-resistant mutant BMPs.

94. The method of claim 88, wherein the first recombinant BMP and the second recombinant BMP are recombinant BMP/BMP-inhibitor fusion proteins.

95. A method for producing a transgenic mammal that upon lactation secretes a recombinant BMP and BMP-inhibitor in its milk, which method comprises breeding a first transgenic mammal, the genome of which comprises a first genetically-engineered nucleic acid sequence, comprising (i) a first nucleic acid sequence encoding a recombinant BMP; (ii) a first mammary gland-specific promoter that directs expression of the recombinant BMP; and (iii) a first signal sequence that provides secretion of the recombinant BMP into the milk of the mammal to a second transgenic mammal, the genome of which comprises a second genetically-engineered nucleic acid sequence, comprising (i) a second nucleic acid sequence encoding a recombinant BMP-inhibitor; and (ii) a second mammary gland-specific promoter that directs expression of the recombinant BMP-inhibitor; and (iii) a second signal sequence that provides secretion of the recombinant BMP-inhibitor into the milk of the mammal.

96. The method of claim 95, wherein the first mammary gland-specific promoter and the second mammary gland-specific promoter are casein promoters.

97. The method of claim 95, wherein the first transgenic animal and the second transgenic animal are goats.

98. The method of claim 95, wherein the recombinant BMP is a recombinant human BMP and the recombinant BMP-inhibitor is a recombinant human BMP-inhibitor.

99. The method of claim 95, wherein the recombinant BMP is a recombinant BMP-2 and the recombinant BMP-inhibitor is a recombinant Noggin.

100. The method of claim 95, wherein the recombinant BMP is a recombinant BMP-7 and the recombinant BMP-inhibitor is a recombinant Sclerostin.
